(19) United States
(12) Patent Application Publication
(10) Pub. No.: US 2009/0131444 A1
(43) Pub. Date: May 21, 2009

(54) Aminopiperidine Quinolines and Their Azaisosteric Analogues with Antibacterial Activity

(75) Inventors: Folkert Reck, Sudbury, MA (US); Marshall Morningstar, Framingham, MA (US); Hajnalka Hartl, Lowell, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Södertälje (SE)

(21) Appl. No.: 11/914,799

(22) PCT Filed: May 23, 2006
(86) PCT No.: PCT/GB2006/001889

§ 371 Date: Nov. 30, 2007

Related U.S. Application Data
(60) Provisional application No. 60/684,030, filed on May 24, 2005.

(51) Int. Cl. A61K 31/517; A61K 31/4709; A61K 31/502; A61K 31/4745; A61K 31/445; C07D 475/02; C07D 471/02; C07D 403/02; C07D 401/02; A61K 31/525

(52) U.S. Cl. 514/248; 514/249; 514/266.21; 514/307; 514/314; 514/300; 514/319; 514/251; 544/235; 544/237; 544/257; 544/350; 544/284; 544/353; 546/148; 546/169; 546/122; 546/205

(57) ABSTRACT

The present invention relates to compounds that demonstrate antibacterial activity, processes for their preparation, pharmaceutical compositions containing them as the active ingredient, to their use as medicaments and to their use in the manufacture of medicaments for use in the treatment of bacterial infections in warm blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm blooded animals such as humans.

### BACKGROUND OF THE INVENTION

The international health community continues to express serious concern that the evolution of antibacterial resistance will continue to result in strains against which currently available antibacterial agents will be ineffective. For example, resistant strains of Gram-positive pathogens such as methicillin-resistant Staphylococcus aureus (MRSA), methicillin-resistant coagulase-negative staphylococci (MRCNS), penicillin-resistant Streptococcus pneumoniae and multiple resistant Enterococcus faecium are both difficult to treat and difficult to eradicate. Consequently, in order to overcome the threat of widespread multi-drug resistant organisms, there is an on-going need to develop new antibiotics, particularly those with either a novel mechanism of action and/or containing new pharmacophoric groups.

### SUMMARY OF THE INVENTION

These and other needs are met by the invention disclosed herein which is directed to a compound formula I:

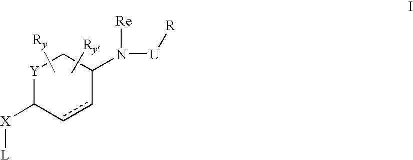

or a pharmaceutically acceptable salt thereof, wherein:

L is an optionally substituted ortho-fused bicyclic subunit with the following structure

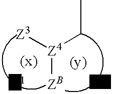

in which:

each ring (x) and (y) contains 0-3 heteroatoms;

at least one of rings (x) and (y) is aromatic;

at least one of Z⁴ and Z⁵ is C;

Z³ is CO; CR₁; N; NRₓ; O; S(O)x wherein x is 0, 1, or 2; SR_{z}, wherein R_{z} is H or (C₁-C₆)alkyl, or CR₁R₁ₐ, provided that R₁ and R₁ₐ are not both OH or optionally substituted amino;

wherein Rₓ is independently hydrogen; (C₁-C₄)alkyl optionally substituted by hydroxy, (C₁-C₆)alkoxycarbonyl, (C₁-C₆)alkoxy, (C₁-C₆)alkylthio, halo or trifluoromethyl; (C₂-C₄)alkenyl; aryl; aryl(C₁-C₄)alkyl; arylcarbonyl; heteroarylcarbonyl; (C₁-C₄)alkoxycarbonyl; (C₁-C₄)alkylcarbonyl; formyl; (C₁-C₆)alkylsulphonyl; or aminocarbonyl wherein the amino group is optionally substituted by (C₁-C₄)alkoxycarbonyl, (C₁-C₄)alkylcarbonyl, (C₂-C₄)alkenyloxycarbonyl, (C₂-C₄)alkenylcarbonyl, (C₁-C₄)alkyl or (C₂-C₄)alkenyl and optionally further substituted by (C₁-C₄)alkyl or (C₂-C₄)alkenyl;

R₁ and R₁ₐ are each independently hydrogen; hydroxy; halogen; (C₁-C₆)alkoxy; (C₁-C₆)alkoxy optionally substituted by (C₁-C₆)alkoxy, amino, piperidyl, guanidino or amidino, any of which is optionally N-substituted by one or two (C₁-C₆)alkyl, acyl or (C₁-C₆)alkylsulphonyl groups, CONH₂, hydroxy, (C₁-C₆)alkylthio, heterocyclylthio, heterocyclyloxy, arylthio, aryloxy, acylthio, acyloxy or (C₁-C₆)alkylsulphonyloxy; (C₁-C₆)alkoxy-substituted (C₁-C₆)alkyl; hydroxy(C₁-C₆)alkyl; halogen; (C₁-C₆)alkyl; (C₁-C₆)alkylthio; trifluoromethyl; trifluoromethoxy; cyano; (C₁-C₆)alkylsulphonyl; (C₁-C₆)alkylsulphoxide; arylsulphonyl; arylsulphoxide; or an amino, piperidyl, guanidino or amidino group optionally N-substituted by one or two (C₁-C₆)alkyl, acyl or (C₁-C₆)alkylsulphonyl groups, or when Z³ and the adjacent linker Z¹ is CR₁CR^{1a}, R₁ and R^{1a} taken together with the carbons to which they are attached may form (C₁-C₂)alkylenedioxy;

Z¹ and Z² are each independently a 2 or 3 atom linker selected from the group consisting of CR_{1b}CR_{1c}N, NCR_{1d}N, CR_{1b}CR_{1c}CR^{1d}, CR_{1d}NN, CR_{1b}NCR_{1c}, NCR_{1b}CR^{1c}OCR_{1b}CR^{1d}, SCR_{1b}CR^{1c}, S(O)ₓCR_{1b}CR^{1c}, SR_{1b}CR_{1b}CR_{1c}, NR_{1b}CR_{1b}CR_{1c}, CR_{1b}N, NNR^{1b}, or S(O)ₓCR_{1b}CR_{1c}, wherein x is 0, 1, or 2;

wherein R^{1b}, R^{1c}, and R^{1d} are each independently hydrogen, hydroxy; halo(C₁-C₆)alkoxy; (C₁-C₆)alkoxy optionally substituted by (C₁-C₆)alkoxy, amino, piperidyl, guanidino or amidino, any of which is optionally N-substituted by one or two (C₁-C₆)allyl, acyl or (C₁-C₆)alkylsulphonyl groups, CONH₂, hydroxy, (C₁-C₆)alkylthio, heterocyclylthio, heterocyclyloxy, arylthio, aryloxy, acylthio, acyloxy or (C₁-C₆)alkylsulphonyloxy; (C₁-C₆)alkoxy-substituted (C₁-C₆)alkyl; hydroxy(C₁-C₆)alkyl; halogen; (C₁-C₆)alkyl; (C₁-C₆)alkylthio; trifluoromethyl; trifluoromethoxy; cyano; carboxy; nitro; azido; acyl; acyloxy; acylthio; (C₁-C₆)alkylsulphonyl; (C₁-C₆)alkylsulphoxide; arylsulphonyl; arylsulphoxide; or an amino, piperidyl, guanidino or amidino group optionally substituted by one or two (C₁-C₆)alkyl, acyl or (C₁-C₆)alkylsulphonyl groups;

X is NR₁₁CO, CO—CR₈R₉, CR₆R₇—CO, O—CR₈R₉, NHR₁₁—CR₈R₉, NR₁₁SO₂, CR₆R₇—SO₂ or CR₆R₇—CR₈R₉,

wherein each of R₆, R₇, R₈ and R₉ is independently selected from: hydrogen; (C₁-C₆)alkoxy; (C₁-C₆)alkylthio; halogen; hydroxyl; halo(C₁-C₆)alkyl; azido; (C₁-C₆)alkyl; (C₂-C₆)alkenyl; (C₁-C₆)alkoxycarbonyl; (C₁-C₆)alkylcarbonyl; (C₂-C₆)alkenyloxycarbonyl; (C₂-C₆)alkenylcarbonyl; trifluoromethoxy; cyano; carboxy; nitro; acyl; acyloxy; acylthio; piperidyl, guanidino or amidino group optionally N-substituted by one or two (C₁-C₆)alkyl, acyl or (C₁-C₆)alkylsulphonyl groups; hydroxy, optionally substituted amino or optionally substituted aminocarbonyl; (C₁-C₆)alkylsulphonyl; (C₂-C₆) alkenylsulphonyl; or (C₁-C₆)aminosulphonyl wherein the amino group is optionally substituted by (C₁-C₆)alkyl or (C₂-C₆)alkenyl; or R₆ and R₈ together represent a bond and R₇ and R₉ are as above defined;

each R₁₁ is independently hydrogen; trifluoromethyl; (C₁-C₆)alkyl; (C₂-C₆)alkenyl; (C₁-C₆)alkoxycarbonyl; (C₁-C₆)alkylcarbonyl; or aminocarbonyl wherein the amino group is optionally substituted by (C₁-C₆)alkoxycarbonyl, (C₁-C₆) alkylcarbonyl, (C₂-C₆)alkenyloxycarbonyl, (C₂-C₆)alkenylcarbonyl, (C₁-C₆)alkyl or (C₂-C₆)alkenyl and optionally further substituted by (C₁-C₆)alkyl or (C₂-C₆)alkenyl; or where one of R₆, R₇, R₈ or R₉ contains a carboxy group and the other contains a hydroxy or amino group they may together form a cyclic ester or amide linkage;

[symbol] is a bond or is absent;

Y is O or NR_{d}, wherein R_{d} is H, (C₁-C₆)alkyl, carboxy(C₁-C₆)alkyl, (C₂-C₆)alkenyl

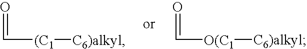

hydroxy(C₁-C₃)alkyl, CONH₂, CO₂H, —CH₂CH₂CO₂H, —CH₂CONH₂, —CH₂CO₂H, —CONH(C₁-C₆)alkyl, trifluoromethyl, S(O)ₓR₁, wherein x is 0, 1, or 2;

Ry and Ry′ are each independently H, halogen, (C₁-C₆)alkyl, hydroxyl, CONH₂, CO₂H, —CH₂CONH₂, —CH₂CO₂H, —CONHCH₃ or amino, provided that when Ry and Ry′ are hydroxyl, amino, or halogen, they are not attached to the same carbon, or when Ry and Ry′ are attached to the same carbon, they form C═O;

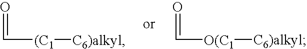

Rₑ is H, (C₁-C₆)alkyl,

U is CH₂, CO, SO₂, or optionally substituted ethylene, or optionally substituted ethenyl, or ethynyl;

R is optionally substituted phenyl or ortho-fused bicyclic aryl or heteroaryl, or

when U is ethyl, ethenyl, or ethynyl, R is hydrogen, halogen, hydroxyl, (C₁-C₆)alkyoxy, (C₁-C₆)alkylthio, (C₁-C₆)alkylsulfinyl, (C₁-C₆)alkylsulfonyl, (C₁-C₆)alkylamino, (C₁-C₆)dialkylamino, (C₃-C₆)cycloalkyl, (C₃-C₆)cyclo(C₁-C₆)alkyloxy, (C₃-C₆)cyclo(C₁-C₆)alkylthio, (C₃-C₆)cyclo(C₁-C₆)alkylsulfinyl, (C₃-C₆)cyclo(C₁₋₆)alkylsulfonyl, (C₃-C₆)cyclo(C₁-C₆)alkylamino, N—(C₃-C₆)cycloalkyl-N—(C₁-C₆)alkylamino, N—(C₃-C₆)(cycloalkyl)₂, (C₁-C₆)acyl, (C₃-C₆)cycloalkylcarbonyl, phenyl, phenoxy, phenylthio, phenylsulfinyl, phenylsulfonyl, phenylamino, N—(C₁-C₆)alkyl-N-phenylamino, N—(C₃-C₆)cycloalkyl-N-phenylamino, N-(phenyl)₂, phenyl(C₁-C₆)alkyloxy, phenyl(C₁-C₆)alkylthio, phenyl(C₁-C₆)alkylsulfinyl, phenyl(C₁-C₆)alkylsulfonyl, phenyl(C₁-C₆)alkylamino, N—(C₁-C₆)alkyl-N-phenylamino(C₁-C₆)alkyl, N—(C₃-C₆)cycloalkyl-N-phenyl(C₁-C₆)alkylamino, benzoyl, mono- or bicyclic heteroaryl, heteroaryloxy, heteroarylthio, heteroarylsulfinyl, heteroarylsulfonyl, heteroarylamino, N—(C₁-C₆)alkyl-N-heteroarylamino, N—(C₃-C₆)cycloalkyl-N-heteroarylamino, heteroarylcarbonyl, heteroaryl(C₁-C₆)alkyloxy, heteroaryl(C₁-C₆)alkylthio, heteroaryl(C₁-C₆)alkylsulfinyl, heteroaryl(C₁-C₆)alkylsulfonyl, heteroaryl(C₁-C₆)alkylamino, N—(C₁-C₆)alkyl-N-heteroarylamino(C₁-C₆)alkyl, N—(C₃-C₆)cycloalkyl-N-heteroarylamino(C₁-C₆)alkyl, carboxyl, (C₁-C₆)alkyloxycarbonyl, —NRₐR_{b} or —CONRₐR_{b} wherein Rₐ and R_{b} respectively represent hydrogen, (C₁-C₆)alkyl, (C₃-C₆)cycloalkyl, phenyl, mono- or bicyclic heteroaryl, or one of Rₐ or R_{b} represents hydroxyl, (C₁-C₆)alkyloxy, (C₃-C₆)cyclo(C₁-C₆)alkyloxy, or Rₐ and R_{b} form together with the nitrogen atom to which they are attached a 5- or 6-membered heterocycle which may optionally contain another heteroatom chosen from O, S and N and carrying, where appropriate, an alkyl, phenyl or mono- or bicyclic heteroaryl substituent on the nitrogen atom or where appropriate in which the sulfur atom is oxidized to the sulfinyl or sulfonyl state), or alternatively

R represents —CR^{o}b=CR^{o}c-R^{o}a wherein R^{o}a is phenyl, phenyl(C₁-C₆)alkyl, heteroaryl or heteroaryl(C₁-C₆)alkyl wherein the heteroaryl part is mono- or bicyclic, phenoxy(C₁-C₆)alkyl, phenylthio(C₁-C₆)alkyl, phenylsulfinyl(C₁-C₆)alkyl, phenylsulfonyl(C₁-C₆)alkyl, phenylamino(C₁-C₆)alkyl, N—(C₁-C₆)alkyl-N-phenylamino(C₁-C₆)alkyl, heteroaryloxy(C₁-C₆)alkyl, heteroarylthio(C₁-C₆)alkyl, heteroarylsulfinyl, heteroarylsulfonyl(C₁-C₆)alkyl, heteroarylamino(C₁-C₆)alkyl, N—(C₁-C₆)alkyl-N-heteroarylamino(C₁-C₆)alkyl, heteroarylthio, heteroarylsulfinyl, heteroarylsulfonyl, phenylthio, phenylsulfinyl, phenylsulfonyl, and wherein R^{o}b and R^{o}c represent hydrogen, (C₁-C₆)alkyl or (C₃-C₆)cycloalkyl, or alternatively R represents a radical C≡C—R^{o}d for which R^{o}d is (C₁-C₆)alkyl, phenyl, phenyl(C₁-C₆)alkyl, phenoxy(C₁-C₆)alkyl, phenylthio(C₁-C₆)alkyl, N—(C₁-C₆)alkyl-N-phenylamino(C₁-C₆)alkyl, mono- or bicyclic heteroaryl, heteroarylalkyl, heteroaryloxy(C₁-C₆)alkyl, heteroarylthio(C₁-C₆)alkyl, heteroarylamino(C₁-C₆)alkyl, N—(C₁-C₆)alkyl-N-heteroarylamino(C₁-C₆)alkyl, or alternatively

R is a radical —CF₂-phenyl or mono- or bicyclic —CF₂-heteroaryl, it being understood that the phenyl, benzyl, benzoyl or heteroaryl radicals or portions mentioned above are optionally substituted on the ring with 1 to 4 substituents chosen from halogen, hydroxyl, alkyl, alkyloxy, alkyloxyalkyl, haloalkyl, trifluoromethyl, trifluoromethoxy, trifluorormethylthio, carboxyl, (C₁-C₆)alkyloxycarbonyl, cyano, alkylamino, —NRₐR_{b} for which Rₐ and R_{b} are as defined above, phenyl, hydroxy(C₁-C₆)alkyl, (C₁-C₆)alkylthio(C₁-C₆)alkyl, (C₁-C₆)alkylsulfinyl(C₁-C₆)alkyl, (C₁-C₆)alkylsulfonyl(C₁-C₆)alkyl.

What is also provided is a compound of formula II

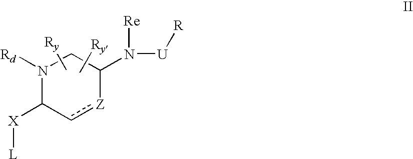

or a pharmaceutically acceptable salt thereof, wherein:

L is

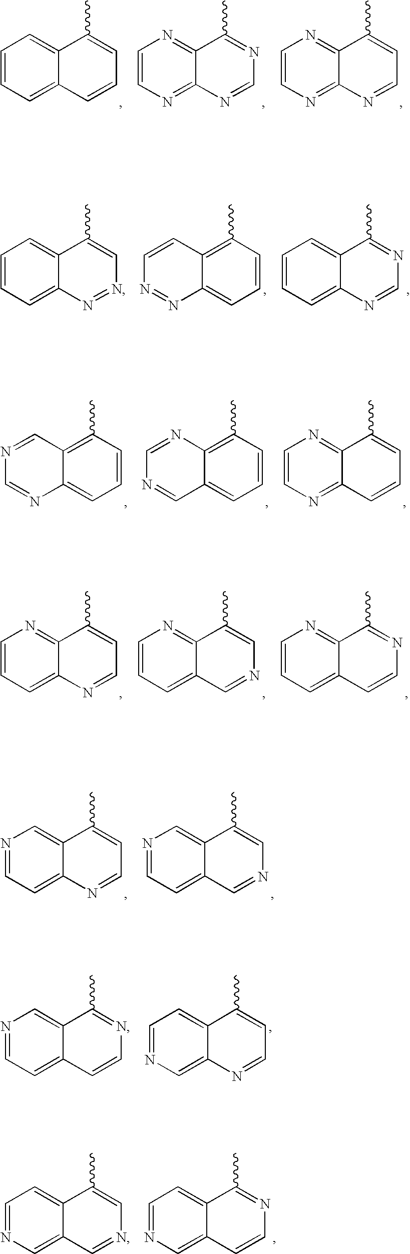

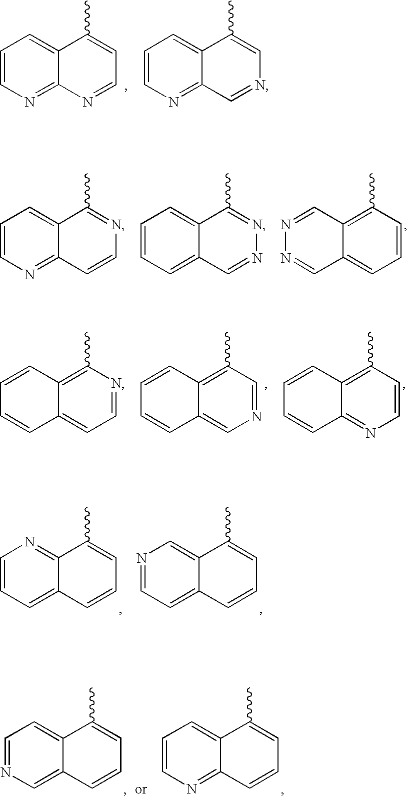

wherein [symbol] indicates the point of attachment, and wherein

each L is optionally substituted with 1, 2, or 3 groups independently selected from the group consisting of H, halo, cyano, nitro, (C₁-C₆)alkanoyl, carboxy, (C₁-C₆)alkoxycarbonyl, (C₁-C₆)alkyl, hydroxyl, halo(C₁-C₆)alkyl, halo(C₁-C₆)alkoxy, (C₁-C₆)alkoxy, NHCO—(C₁-C₆)alkyl, SO₂(C₁-C₆)alkyl, SO₂NH(C₁-C₆)alkyl, or SO₂N((C₁-C₆)alkyl)₂;

X is NHCO, N(C₁-C₆)alkylCO, CO—CR₁R₂, CR₁R₂—CO, NR₁SO₂, CR₁R₂—SO₂ or CR₁R₂—CR₁R₂, wherein R₁ and R₂ at each occurrence is independently H, hydroxyl, (C₁-C₆)alkyl, halogen, halo(C₁-C₆)alkyl, aryl, or heteroaryl; or

X is O—CR₁R₂, NR₁—CR₁R₂, wherein R₁ and R₂ are H, (C₁-C₆)alkyl, halo(C₁-C₆)alkyl, aryl, or heteroaryl;

Z is absent or is C;

[symbol] is a bond or is absent;

R_{d} is H, (C₁-C₆)alkyl, (C₂-C₆)alkenyl,

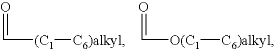

hydroxy(C₁-C₃)alkyl, CONH₂, CO₂H, —CH₂CH₂CO₂H, —CH₂CONH₂, —CH₂CO₂H, —CONH(C₁-C₆)alkyl, trifluoromethyl, S(O)ₓR₁, wherein x is 1 or 2, provided that when R_{d} is H and Z is C, [symbol] is a bond;

Ry and Ry′ are each independently halogen, (C₁-C₆)alkyl, (C₁-C₆)alkoxy, hydroxyl, CONH₂, CO₂H, —CH₂CONH₂, —CH₂CO₂H, —CONHCH₃ or amino, provided that when Ry and Ry′ are hydroxyl, amino, or halogen, they are not attached to the same carbon, or when Ry and Ry′ are attached to the same carbon, they form C═O;

Rₑ is H, (C₁-C₆)alkyl,

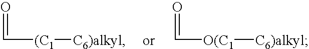

U is CH₂, CH₂CH₂, CH═CH, or C≡C, and wherein each hydrogen may be optionally replaced by fluoro or (C₁-C₆)alkyl;

R is an optionally substituted aryl or ortho-fused bicyclic heteroaryl, or when U is ethylene, ethenyl, or ethynyl, R is optionally substituted aryl or heteroaryl, or is heteroaryl(C₁-C₆)alkyloxy, heteroaryl(C₁-C₆)alkylthio, heteroaryl(C₁-C₆)alkylsulfinyl, heteroaryl(C₁-C₆)alkylsulfonyl, heteroaryl(C₁-C₆)alkylamino.

What is also provided is a compound of claim 1 which is a compound of formula II-1

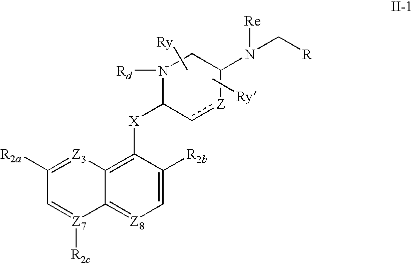

or a pharmaceutically acceptable salt thereof, wherein:

Z₃, Z₇, and Z₈ are C or N provided that when Z₇ is N, R_{2c} is absent, and

R₂ₐ is H, cyano, (C₁-C₆)alkyl, hydroxyl, halo, halo(C₁-C₆)alkoxy, (C₁-C₆)alkoxy.

What is also provided is a compound of formula II-2

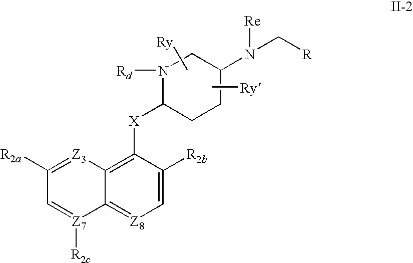

or a pharmaceutically acceptable salt hereof, wherein.

R_{d} is H, (C₁-C₆)alkyl,

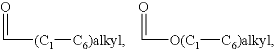

methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

What is also provided is a compound of formula II-3.

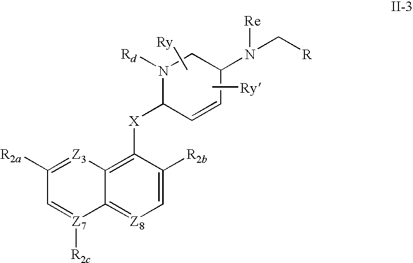

or a pharmaceutically acceptable salt thereof, wherein

R_{d} is H, (C₁-C₆)alkyl,

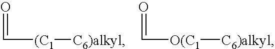

methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

What is also provided is a compound of formula II-4

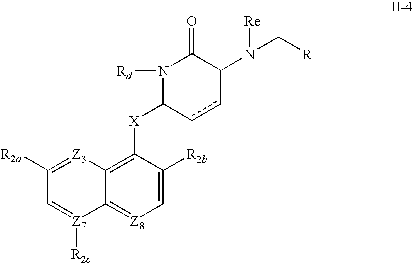

or a pharmaceutically acceptable salt thereof, wherein:

R_{d} is H, (C₁-C₆)alkyl, carboxy(C₁-C₆)alkyl,

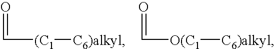

methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

What is also provided is a compound of formula II-5.

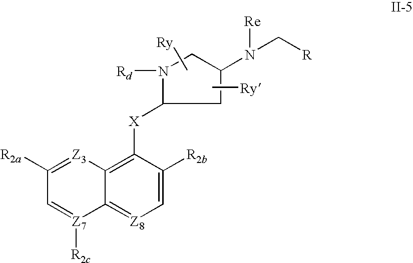

or a pharmaceutically acceptable salt thereof, wherein:

R_{d}H, is (C₁-C₆)alkyl, carboxy(C₁-C₆)alkyl

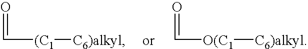

methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

The invention also provides a compound which is:
(2,3-Dihydro[1,4]dioxino[2,3-c]pyridin-7-ylmethyl) {(3R,6R)-6-[2-(3-fluoro-6-methoxy-1,5-naphthyridin-4-yl)ethyl]tetrahydro-2H-pyran-3-yl}amine;
(2S,5R)-5-[(2,3-Dihydro[1,4]dioxino[2,3-c]pyridin-7-ylmethyl)amino]-N-(6-methoxy-1,5-naphthyridin-4-yl)tetrahydro-2H-pyran-2-carboxamide;
((3R,6S)-6-{[(3-Chloro-6-methoxyquinolin-4-yl)oxy]methyl}tetrahydro-2H-pyran-3-yl)(2,3-dihydro[1,4]dioxino[2,3-c]pyridin-7-ylmethyl)amine;
(2S,5R)-5-[(2,3-dihydro[1,4]dioxino[2,3-c]pyridin-7-ylmethyl)amino]-N-(6-methoxy-1,5-naphthyridin-4-yl)piperidine-2-carboxamide;
(2S,5R)-5-[(2,3-dihydro[1,4]dioxino[2,3-c]pyridin-7-ylmethyl)amino]-N-(6-methoxyquinolin-4-yl)piperidine-2-carboxamide;
(2S,5R)—N-(2-cyanoquinolin-8-yl)-5-[(2,3-dihydro[1,4]dioxino[2,3-c]pyridin-7-ylmethyl)amino]piperidine-2-carboxamide;
(2R,5S)—N-(2-cyanoquinolin-8-yl)-5-[(2,3-dihydro[1,4]dioxino[2,3-c]pyridin-7-ylmethyl)amino]piperidine-2-carboxamide;
(2R,5S)-5-[(2,3-dihydro[1,4]dioxino[2,3-c]pyridin-7-ylmethyl)amino]-N-(6-methoxy-1,5-naphthyridin-4-yl)piperidine-2-carboxamide;
N-(2-cyanoquinolin-8-yl)-5-{[(3-oxo-3,4-dihydro-2H-pyrido[3,2-b][1,4]oxazin-6-yl)methyl]amino}piperidine-2-carboxamide;
N-(6-methoxy-1,5-naphthyridin-4-yl)-5-{[(3-oxo-3,4-dihydro-2H-pyrido[3,2-b][1,4]oxazin-6-yl)methyl]amino}piperidine-2-carboxamide;
N-(6-methoxyquinolin-4-yl)-5-{[(3-oxo-3,4-dihydro-2H-pyrido[3,2-b][1,4]oxazin-6-yl)methyl]amino}piperidine-2-carboxamide;
N-(2-cyanoquinolin-8-yl)-5-{[(3-oxo-3,4-dihydro-2H-pyrido[3,2-b][1,4]thiazin-6-yl)methyl]amino}piperidine-2-carboxamide;
N-(6-methoxy-1,5-naphthyridin-4-yl)-5-{[(3-oxo-3,4-dihydro-2H-pyrido[3,2-b][1,4]thiazin-6-yl)methyl]amino}piperidine-2-carboxamide;
N-(6-methoxyquinolin-4-yl)-5-{[(3-oxo-3,4-dihydro-2H-pyrido[3,2-b][1,4]thiazin-6-yl)methyl]amino}piperidine-2-carboxamide;
N-(2-cyanoquinolin-8-yl)-5-{[(2E)-3-(2,5-difluorophenyl)prop-2-en-1-yl]amino}piperidine-2-carboxamide;
5-{[(2E)-3-(2,5-difluorophenyl)prop-2-en-1-yl]amino}-N-(6-methoxy-1,5-naphthyridin-4-yl)piperidine-2-carboxamide;
5-{[(2E)-3-(2,5-difluorophenyl)prop-2-en-1-yl]amino}-N-(6-methoxyquinolin-4-yl)piperidine-2-carboxamide;
6-[2-(3-chloro-6-methoxyquinolin-4-yl)ethyl]-N-(2,3-dihydro[1,4]dioxino[2,3-c]pyridin-7-ylmethyl)piperidin-3-amine;
6-[2-(3-chloro-6-methoxy-1,5-naphthyridin-4-yl)ethyl]-N-(2,3-dihydro[1,4]dioxino[2,3-c]pyridin-7-ylmethyl)piperidin-3-amine;
N-(2,3-dihydro[1,4]dioxino[2,3-c]pyridin-7-ylmethyl)-6-[2-(3-fluoro-6-methoxyquinolin-4-yl)ethyl]piperidin-3-amine;
N-(2,3-dihydro[1,4]dioxino[2,3-c]pyridin-7-ylmethyl)-6-[2-(3-fluoro-6-methoxy-1,5-naphthyridin-4-yl)ethyl]piperidin-3-amine;
6-[({6-[2-(3-chloro-6-methoxyquinolin-4-yl)ethyl]piperidin-3-yl}amino)methyl]-2H-pyrido[3,2-b][1,4]oxazin-3(4H)-one;
6-[({6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
6-[({6-[2-(3-fluoro-6-methoxyquinolin-4-yl)ethyl]piperidin-3-yl}amino)methyl]-2H-pyrido[3,2-b][1,4]oxazin-3(4H)-one;
6-[({6-[2-(3-fluoro-6-methoxy-1,5-naphthyridin-4-yl)ethyl]piperidin-3-yl}amino)methyl]-2H-pyrido[3,2-b][1,4]oxazin-3(4H)-one;
6-[({6-[2-(3-chloro-6-methoxyquinolin-4-yl)ethyl]piperidin-3-yl}amino)methyl]-2H-pyrido[3,2-b][1,4]thiazin-3(4H)-one;
6-[({6-[2-(3-chloro-6-methoxy-1,5-naphthyridin-4-yl)ethyl]piperidin-3-yl}amino)methyl]-2H-pyrido[3,2-b][1,4]thiazin-3(4H)-one;
6-[({6-[2-(3-fluoro-6-methoxyquinolin-4-yl)ethyl]piperidin-3-yl}amino)methyl]-2H-pyrido[3,2-b][1,4]thiazin-3(4H)-one;
6-[({6-[2-(3-fluoro-6-methoxy-1,5-naphthyridin-4-yl)ethyl]piperidin-3-yl}amino)methyl]-2H-pyrido[3,2-b][1,4]thiazin-3(4H)-one;
6-[2-(3-chloro-6-methoxyquinolin-4-yl)ethyl]-N-[(2E)-3-(2,5-difluorophenyl)prop-2-en-1-yl]piperidin-3-amine;
6-[2-(3-chloro-6-methoxy-1,5-naphthyridin-4-yl)ethyl]-N-[(2E)-3-(2,5-difluorophenyl)prop-2-en-1-yl]piperidin-3-amine;
N-[(2E)-3-(2,5-difluorophenyl)prop-2-en-1-yl]-6-[2-(3-fluoro-6-methoxyquinolin-4-yl)ethyl]piperidin-3-amine;
N-[(2E)-3-(2,5-difluorophenyl)prop-2-en-1-yl]-6-[2-(3-fluoro-6-methoxy-1,5-naphthyridin-4-yl)ethyl]piperidin-3-amine;
5-[(2,3-dihydro[1,4]dioxino[2,3-c]pyridin-7-ylmethyl)amino]-N-(3-methoxyquinoxalin-5-yl)piperidine-2-carboxamide;
N-(3-methoxyquinoxalin-5-yl)-5-{[(3-oxo-3,4-dihydro-2H-pyrido[3,2-b][1,4]oxazin-6-yl)methyl]amino}piperidine-2-carboxamide;
N-(3-methoxyquinoxalin-5-yl)-5-{[(3-oxo-3,4-dihydro-2H-pyrido[3,2-b][1,4]thiazin-6-yl)methyl]amino}piperidine-2-carboxamide;
5-{[(2E)-3-(2,6-difluorophenyl)prop-2-enyl]amino}-N-(3-methoxyquinoxalin-5-yl)piperidine-2-carboxamide;
(2S,5R)-5-[(2,3-dihydro-1,4-benzodioxin-6-ylmethyl)amino]-N-(6-methoxy-1,5-naphthyridin-4-yl)piperidine-2-carboxamide;
(2S,5R)—N-(6-methoxy-1,5-naphthyridin-4-yl)-5-{[(3-oxo-3,4-dihydro-2H-pyrido[3,2-b][1,4]oxazin-6-yl)methyl]amino}piperidine-2-carboxamide;
(2S,5R)-5-(benzylamino)-N-(6-methoxy-1,5-naphthyridin-4-yl)piperidine-2-carboxamide;
(4R)-4-[(2,3-dihydro[1,4]dioxino[2,3-c]pyridin-7-ylmethyl)amino]-N-(6-methoxy-1,5-naphthyridin-4-yl)-L-prolinamide;
(4R)—N-(2-cyanoquinolin-8-yl)-4-[(2,3-dihydro[1,4]dioxino[2,3-c]pyridin-7-ylmethyl)amino]-L-prolinamide;
(4R)-4-[(2,3-dihydro[1,4]dioxino[2,3-c]pyridin-7-ylmethyl)amino]-N-(8-fluoro-6-methoxyquinolin-4-yl)-L-prolinamide;
(4R)—N-(6-cyano-1,7-naphthyridin-4-yl)-4-[(2,3-dihydro[1,4]dioxino[2,3-c]pyridin-7-ylmethyl)amino]-L-prolinamide;
6-[({(3S,6S)-6-[2-(3-fluoro-6-methoxy-1,5-naphthyridin-4-yl)ethyl]piperidin-3-yl}amino)methyl]-2H-pyrido[3,2-b][1,4]oxazin-3(4H)-one;
(3S,6S)—N-(2,3-dihydro[1,4]dioxino[2,3-c]pyridin-7-ylmethyl)-6-[2-(3-fluoro-6-methoxy-1,5-naphthyridin-4-yl)ethyl]piperidin-3-amine;
(3S,6S)-6-[2-(3-chloro-6-methoxy-1,5-naphthyridin-4-yl)ethyl]-N-[(2E)-3-(2,5-difluorophenyl)prop-2-en-1-yl]piperidin-3-amine;
6-[({(3S,6S)-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
(3S,6S)-6-[2-(3-chloro-6-methoxy-1,5-naphthyridin-4-yl)ethyl]-N-(2,3-dihydro[1,4]dioxino[2,3-c]pyridin-7-ylmethyl)piperidin-3-amine;
6-[({(3S,6S)-6-[2-(3-chloro-6-methoxy-1,5-naphthyridin-4-yl)ethyl]piperidin-3-yl}amino)methyl]-2H-pyrido[3,2-b][1,4]thiazin-3(4H)-one;
8-[2-((2S,5S)-5-{[(3-oxo-3,4-dihydro-2H-pyrido[3,2-b][1,4]oxazin-6-yl)methyl]amino}piperidin-2-yl)ethyl]quinoline-2-carbonitrile;
8-(2-{(2S,5S)-5-[(2,3-dihydro[1,4]dioxino[2,3-c]pyridin-7-ylmethyl)amino]piperidin-2-yl}ethyl)quinoline-2-carbonitrile;
(3R,6R)-6-[2-(3-chloro-6-methoxy-1,5-naphthyridin-4-yl)ethyl]-N-[(2E)-3-(2,5-difluorophenyl)prop-2-en-1-yl]piperidin-3-amine;
(3R,6R)-6-[2-(3-chloro-6-methoxy-1,5-naphthyridin-4-yl)ethyl]-N-(2,3-dihydro[1,4]dioxino[2,3-c]pyridin-7-ylmethyl)piperidin-3-amine;
6-[({(3R,6R)-6-[2-(3-chloro-6-methoxy-1,5-naphthyridin-4-yl)ethyl]piperidin-3-yl}amino)methyl]-2H-pyrido[3,2-b][1,4]thiazin-3(4H)-one;
6-[({(3R,6R)-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
6-[({(3S,6S)-6-[2-(3-chloro-6-fluoroquinolin-4-yl)ethyl]piperidin-3-yl}amino)methyl]-2H-pyrido[3,2-b][1,4]oxazin-3(4H)-one;
7-chloro-8-[2-((2S,5S)-5-{[(3-oxo-3,4-dihydro-2H-pyrido[3,2-b][1,4]oxazin-6-yl)methyl]amino}piperidin-2-yl)ethyl]-1,5-naphthyridine-2-carbonitrile;
7-fluoro-8-[2-((2R,5R)-5-{[(3-oxo-3,4-dihydro-2H-pyrido[3,2-b][1,4]oxazin-6-yl)methyl]amino}piperidin-2-yl)ethyl]-1,5-naphthyridine-2-carbonitrile;
(3S,6S)—N-(2,3-dihydro[1,4]dioxino[2,3-c]pyridin-7-ylmethyl)-6-[2-(6-methoxy-1,5-naphthyridin-4-yl)ethyl]piperidin-3-amine;
(3S,6S)—N-[(2E)-3-(2,5-difluorophenyl)prop-2-en-1-yl]-6-[2-(6-methoxy-1,5-naphthyridin-4-yl)ethyl]piperidin-3-amine;
6-[({(3S,6S)-6-[2-(6-methoxy-1,5-naphthyridin-4-yl)ethyl]piperidin-3-yl}amino)methyl]-2H-pyrido[3,2-b][1,4]oxazin-3(4H)-one;
(3R,6R)-6-[2-(3-chloro-6-methoxy-1,5-naphthyridin-4-yl)ethyl]-N-(2,3-dihydro[1,4]dioxino[2,3-c]pyridin-7-ylmethyl) 1-methylpiperidin-3-amine;
6-({[(3S,6S)-6-[2-(3-chloro-6-methoxy-1,5-naphthyridin-4-yl)ethyl]-1-(methylsulfonyl)piperidin-3-yl]amino}methyl)-2H-pyrido[3,2-b][1,4]oxazin-3(4H)-one;
6-({[(3R,6R)-6-[2-(3-chloro-6-methoxy-1,5-naphthyridin-4-yl)ethyl]-1-(methylsulfonyl)piperidin-3-yl]amino}methyl)-2H-pyrido[3,2-b][1,4]oxazin-3(4H)-one;
6-[({(3S,6S)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
6-[({(3R,6R)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetic acid;
((2S,5S)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
((2R,5R)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
6-({[(3S,6S)-6-[2-(3-chloro-6-methoxy-1,5-naphthyridin-4-yl)ethyl]-1-(methoxyacetyl)piperidin-3-yl]amino}methyl)-2H-pyrido[3,2-b][1,4]oxazin-3(4H)-one;
2-((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)-2-oxoethyl acetate;
6-[({(3S,6S)-6-[2-(3-chloro-6-methoxy-1,5-naphthyridin-4-yl)ethyl]-1-glycoloylpiperidin-3-yl}amino)methyl]-2H-pyrido[3,2-b][1,4]oxazin-3(4H)-one; or
(3R,6S)—N-(2,3-dihydro[1,4]dioxino[2,3-c]pyridin-7-ylmethyl)-6-[2-(3-fluoro-6-methoxy-1,5-naphthyridin-4-yl)ethyl]piperidin-3-amine.

The invention also provides a compound which is:
(2S,5R)-5-[(2,3-dihydro-1,4-benzodioxin-6-ylmethyl)amino]-N-(6-methoxy-1,5-naphthyridin-4-yl)-6-oxopiperidine-2-carboxamide;
5-[(2,3-dihydro-1,4-benzodioxin-6-ylmethyl)amino]-N-(6-methoxy-1,5-naphthyridin-4-yl)-6-oxopiperidine-2-carboxamide;
(2S,5S)-5-[(2,3-dihydro-1,4-benzodioxin-6-ylmethyl)amino]-N-(6-methoxy-1,5-naphthyridin-4-yl)-6-oxopiperidine-2-carboxamide;
(4R)-4-[(2,3-dihydro[1,4]dioxino[2,3-c]pyridin-7-ylmethyl)amino]-N-(8-fluoro-6-methoxyquinolin-4-yl)-L-prolinamide;
(4R)—N-(6-cyano-1,7-naphthyridin-4-yl)-4-[(2,3-dihydro[1,4]dioxino[2,3-c]pyridin-7-ylmethyl)amino]-L-prolinamide;
(4R)-4-[(2,3-dihydro[1,4]dioxino[2,3-c]pyridin-7-ylmethyl)amino]-N-(6-methoxy-1,5-naphthyridin-4-yl)-L-prolinamide;
(4R)—N-(2-cyanoquinolin-8-yl)-4-[(2,3-dihydro[1,4]dioxino[2,3-c]pyridin-7-ylmethyl)amino]-L-prolinamide;
(3R,6R)-6-[2-(3-chloro-6-methoxy-1,5-naphthyridin-4-yl)ethyl]-N-(2,3-dihydro[1,4]dioxino[2,3-c]pyridin-7-ylmethyl)-1-methylpiperidin-3-amine;
6-[({(3S,6S)-6-[2-(3-chloro-6-methoxy-1,5-naphthyridin-4-yl)ethyl]-1-glycoloylpiperidin-3-yl}amino)methyl]-2H-pyrido[3,2-b][1,4]oxazin-3(4H)-one;
6-[({(3R,6R)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4R)-one;
((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetic acid;
6-[({(3S,6S)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
((2R,5R)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
6-({[(3S,6S)-6-[2-(3-chloro-6-methoxy-1,5-naphthyridin-4-yl)ethyl]-1-(methoxyacetyl)piperidin-3-yl]amino}methyl)-2H-pyrido[3,2-b]L[1,4]oxazin-3(4H)-one;
((2S,5S)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
tert-butyl (2R,5R)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidine-1-carboxylate;
2-((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)-2-oxoethyl acetate; or
(2S,5S)-5-[(2,3-dihydro-1,4-benzodioxin-6-ylmethyl)(methyl)amino]-N-(6-methoxy-1,5-naphthyridin-4-yl)-1-methylpiperidine-2-carboxamide,

or a pharmaceutically acceptable salt thereof.

The invention also provides a pharmaceutical composition comprising a compound of formulas I, II, II-1, II-2, II-3, II-4, and II-5 admixed with a pharmaceutically acceptable adjuvant, carrier, or excipient.

The invention also provides a method of treating a bacterial infection comprising administering a therapeutically effective amount of a compound of formulas I, II, II-1, II-2, II-3, II-4, and II-5 to a mammal in need thereof.

The invention also provides a method of treating a bacterial infection in a warm-blooded animal, such as a human being, in need of such treatment, which comprises administering to said animal an effective amount of a compound of formulas I, II, II-1, II-2, II-3, II-4, and II-5 or a pharmaceutically-acceptable salt thereof.

The invention also provides a method for inhibiting bacterial DNA gyrase in a warm-blooded animal, such as a human being, in need of such treatment which comprises administering to said animal an effective amount of a compound of formulas I, II, II-1, II-2, II-3, II-4, and II-5 or a pharmaceutically acceptable salt.

The invention also provides a compound of formulas I, II, II-1, II-2, II-3, II-4, and II-5 and pharmaceutically acceptable salts thereof for use as a medicament.

The invention also provides the use of a compound of formulas I, II, II-1, II-2, II-3, II-4, and II-5 or a pharmaceutically acceptable salt thereof in the manufacture of a medicament for use in the production of an anti-bacterial effect in a warm-blooded animal such as a human being.

The invention also provides the use of a compound of formulas I, II, II-1, II-2, II-3, II-4, and II-5 or a pharmaceutically acceptable salt thereof in the manufacture of a medicament for use in the treatment of a bacterial infection in a warm-blooded animal such as a human being.

The invention also provides a process for preparing a compound of formulas I, II, II-1, II-2, II-3, II-4, and II-5 comprising one of the following approaches:

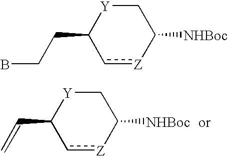

(a) Pd-catalyzed coupling of

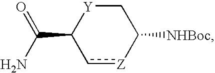

wherein Y is N-PG, wherein PG is a protecting group, with

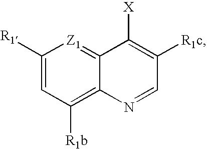

wherein X is a leaving group selected from halo or trifluoromethylsulfonyloxy, followed by removal of the BOC group and addition of U—R via reductive amination;

### (b) Coupling of

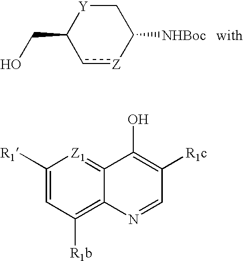

under Mitsunobu conditions followed by removal of the BOC group and addition of U—R via reductive amination; or
(c) Amide formation using

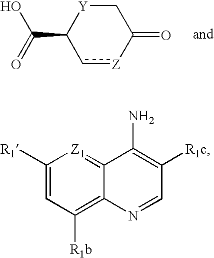

followed by addition of U—R via reductive amination.

### DETAILED DESCRIPTION OF THE INVENTION

Unless otherwise stated, the following terms used in the specification and claims have the following meanings.

### DEFINITIONS

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, pentyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like.

“Alkenylene” means a linear divalent hydrocarbon radical of two to six carbon atoms or a branched divalent hydrocarbon radical of three to six carbon atoms, containing at least one double bond, e.g., ethenylene, 2,4-pentadienylene, and the like.

“Alkynyl” means an alkyl group having one or more carbon-carbon triple bonds, e.g., ethynyl.

“Acyl” means a radical —C(O)R where R is hydrogen, alkyl, alkenyl, cycloalkyl, heteroalkyl, haloalkyl, aryl, aralkyl, heteroaralkyl or heteroaryl, e.g., acetyl, benzoyl, thienoyl, and the like.

“Acyloxy” means a radical —OC(O)R where R is hydrogen, alkyl, alkenyl, cycloalkyl, heteroalkyl, haloalkyl or optionally substituted phenyl, e.g., acetoxy, benzoyloxy, and the like.

“Acylthio” means a radical —SC(O)R where R is hydrogen, alkyl, alkenyl, cycloalkyl, heteroalkyl, haloalkyl or optionally substituted phenyl, e.g., thioacetoxy, thiobenzoyloxy, and the like.

“Halo” means fluoro, chloro, bromo or iodo.

“Haloalkyl” means alkyl substituted with one or more same or different halo atoms, e.g., —CH₂Cl, —CF₃, —CH₂CF₃, —CH₂CCl₃, and the like.

“Cycloalkyl” means a saturated monovalent cyclic hydrocarbon radical of three to six ring carbons, e.g., cyclopropyl, cyclohexyl, and the like.

“Cycloalkyloxy” means a cycloalkyl-O— group in which the cycloalkyl group is as described herein, and includes, e.g., cycloalkyloxy groups include cyclopropyloxy, cyclopentyloxy, cyclohexyloxy and cycloheptyloxy.

“Cycloalkylthio” means a cycloalkyl-S— group in which the cycloalkyl group is as described herein, and includes, e.g., cycloalkylthio groups include cyclopropylthio, cyclopentylthio, cyclohexylthio and cycloheptylthio.

“Carbocycle” means a saturated, cyclic group of 3 to 6 ring atoms in which all the ring atoms are carbon, e.g., cyclopentyl, cyclohexyl, and the like.

“Amine” or “amino” refers to radicals of the general formula —NRR′, wherein R and R′ are independently selected from hydrogen or a hydrocarbyl radical, or wherein R and R′ combined form a heterocycle. Examples of amino groups include: —NH₂, methyl amino, diethyl amino, anilino, benzyl amino, piperidinyl, piperazinyl and indolinyl.

“Monosubstituted-amino” means a radical —NHR where R is alkyl, heteroalkyl, haloalkyl, cycloalkyl, cycloalkylalkyl or optionally substituted phenyl, e.g., methylamino, (1-methylethyl)amino, phenylamino, and the like.

“Disubstituted-amino” means a radical —NRR′ where R and R′ are independently alkyl, alkenyl, heteroalkyl, haloalkyl, cycloalkyl, cycloalkylalkyl or optionally substituted phenyl. Representative examples include, but are not limited to, dimethylamino, methylethylamino, di(1-methylethyl)amino, methylbenzylamino, and the like.

“Aryl” means a monovalent monocyclic or bicyclic aromatic hydrocarbon radical of 6 to 10 ring atoms, and optionally substituted independently with one or more substituents, preferably one, two or three substituents selected from alkyl, haloalkyl, heteroalkyl, cycloalkyl, cycloalkylalkyl, halo, cyano, nitro, acyloxy, alkoxy, optionally substituted phenyl, heteroaryl, heteroaralkyl, amino, monosubstituted amino, disubstituted amino, acylamino, hydroxylamino, amidino, guanidino, cyanoguanidinyl, hydrazino, hydrazido, —OR [where R is hydrogen, alkyl, haloalkyl, alkenyl, cycloalkyl, cycloalkylalkyl, optionally substituted phenyl, heteroaryl or heteroaralkyl], —S(O)ₙR [where n is an integer from 0 to 2 and R is hydrogen, alkyl, haloalkyl, alkenyl, cycloalkyl, cycloalkylalkyl, optionally substituted phenyl, heteroaryl, heteroaralkyl, amino, mono or disubstituted amino], —NRSO₂R′ (where R is hydrogen or alkyl and R′ is alkyl, amino, monosubstituted or disubstituted amino) —C(O)R (where R is hydrogen, alkyl, alkenyl, cycloalkyl, heteroalkyl, haloalkyl or optionally substituted phenyl), —COOR (where R is hydrogen, alkyl, optionally substituted phenyl, heteroaryl or heteroaralkyl), -(alkylene)-COOR (where R is hydrogen, alkyl, optionally substituted phenyl, heteroaryl or heteroaralkyl), methylenedioxy, 1,2-ethylenedioxy, —CONR′R″ or -(alkylene)CONR′R″ (where R′ and R″ are independently selected from hydrogen, alkyl, cycloalkyl, haloalkyl, cycloalkylalkyl, optionally substituted phenyl, heteroaryl and heteroaralkyl). More specifically the term aryl includes, but is not limited to, phenyl, 1-naphthyl, 2-naphthyl, and derivatives thereof.

The term “ortho-fused” as used in the phrase “ortho-fused bicyclic subunit” means a bicyclic saturated, partially aromatic, or fully aromatic carbocyclic or heterocyclic ring system wherein the two rings have only two atoms and one bond in common. Both rings may be aromatic; for example, such as in naphthalene, pteridine, cinnoline, quinazoline, quinoxaline, naphthyridine, phthalazine, quinoline, isoquinoline, quinolizine, purine, indazole, indole, isoindole, indolizine, or pyrrolizine and the like.

“Heteroaryl” means a monovalent monocyclic or bicyclic aromatic radical of 5 to 10 ring atoms containing one, two, or three ring heteroatoms selected from N, O, or S, the remaining ring atoms being C. The aromatic radical is optionally substituted independently with one or more substituents, preferably one or two substituents selected from alkyl, haloalkyl, heteroalkyl, cycloalkyl, cycloalkylalkyl, halo, cyano, nitro, acyloxy, optionally substituted phenyl, amino, monosubstituted amino, disubstituted amino, acylamino, hydroxyamino, amidino, guanidino, cyanoguanidinyl, hydrazino, hydrazido, —OR [where R is hydrogen, alkyl, haloalkyl, alkenyl, cycloalkyl, cycloalkylalkyl or optionally substituted phenyl], —S(O)ₙR [where n is an integer from 0 to 2 and R is hydrogen, alkyl, haloalkyl, alkenyl, cycloalkyl, cycloalkylalkyl, optionally substituted phenyl, amino, mono or disubstituted amino], —C(O)R (where R is hydrogen, alkyl, alkenyl, cycloalkyl, heteroalkyl, haloalkyl or optionally substituted phenyl), —COOR (where R is hydrogen, alkyl, or optionally substituted phenyl), -(alkylene)-COOR (where R is hydrogen, alkyl or optionally substituted phenyl), methylenedioxy, 1,2-ethylenedioxy, —CONR′R″ or -(alkylene)-CONR′R″ (where R′ and R″ are independently selected from hydrogen, alkyl, cycloalkyl, haloalkyl, cycloalkylalkyl or optionally substituted phenyl). The term heteroaryl includes, but is not limited to pyridyl, pyrrolyl, thiophene, pyrazolyl, thiazolyl, imidazolyl, pyrimidinyl, thiadiazolyl, indolyl, carbazolyl, azaindolyl, benzofuranyl, benzotriazolyl, benzisoxazolyl, purinyl, quinolinyl, benzopyranyl, and derivatives thereof.

“Heterocycle” or “Heterocyclyl” means a saturated or unsaturated cyclic radical of 3 to 8 ring atoms in which one or two ring atoms are heteroatoms selected from N, O, or S(O).sub.n (where n is an integer from 0 to 2). The heterocyclo ring may be optionally substituted independently with one, two or three substituents selected from alkyl, haloalkyl, cycloalkyl, cycloalkylalkyl, aryl, aralkyl, heteroaryl, heteroaralkyl, halo, cyano, acyl, acylamino, amino, monosubstituted amino, disubstituted amino, —COOR (where R is hydrogen or alkyl), —XR (where X is O or S(O), where n is an integer from 0 to 2 and R is hydrogen, alkyl, haloalkyl, cycloalkyl, aralkyl, aryl, heteroaryl or heteroaralkyl) or —CONR′R″ (where R′ and R″ are independently selected from hydrogen or alkyl). Representative examples include, but are not limited to tetrahydropyranyl, piperidino, 1-(4-chlorophenyl)piperidino, and the like.

“Heterocyclylalkyl” means a radical -Rₐ-R_{b} where Rₐ is bound to R_{b} and Rₐ is an alkylene group and R_{b} is a heterocyclyl group as defined above e.g., tetrahydropyran-2-ylmethyl, 4-methylpiperazin-1-ylethyl, and the like.

“Heterocyclylthio” means a radical -Rₐ-R_{b} where Rₐ is bound to R_{b} and Rₐ is a thio group and R_{b} is a heterocyclyl group as defined above.

“Heterocyclyloxy” means a radical -Rₐ-R_{b} where Rₐ is bound to R_{b} and Rₐ is an oxygen and R_{b} is a heterocyclyl group as defined above.

“Arylalkyl” or “aralkyl” means a radical -RₐR_{b} where Rₐ is bound to R_{b} and Rₐ is an alkylene group and R_{b} is an aryl group as defined above e.g., benzyl, phenylethyl, 3-(3-chlorophenyl)-2-methylpentyl, and the like.

“Aryloxy” means an aryl-O— group in which the aryl group is as previously described. Exemplary aryloxy groups include optionally substituted phenoxy and naphthoxy.

“Arylthio” means an aryl-S— group in which the aryl group is as previously described. Exemplary arylthio groups include phenylthio and naphthylthio.

“Heteroaralkyl” means a radical -Rₐ-R_{b} where Rₐ is bound to R_{b} and Rₐ is an alkylene group and R_{b} is a heteroaryl group as defined above e.g., pyridin-3-ylmethyl, 3-(benzofuran-2-yl)propyl, and the like.

“Hydroxyalkyl” means a linear monovalent hydrocarbon radical of two to six carbon atoms or a branched monovalent hydrocarbon radical of three or six carbons substituted with one or two hydroxy groups, provided that if two hydroxy groups are present they are not both on the same carbon atom. Representative examples include, but are not limited to, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Alkoxy”, “haloalkyloxy”, “aryloxy”, “heteroaryloxy”, “aralkyloxy”, or “heteroaralkyloxy” means a radical —OR where R is an alkyl, haloalkyl, aryl, heteroaryl, respectively as defined above e.g., methoxy, phenoxy, pyridin-2-yloxy, benzyloxy, and the like. “Optional” or “optionally” means that the subsequently described event or circumstance may but need not occur, and that the description includes instances where the event or circumstance occurs and instances in which it does not. For example, “heterocyclo group optionally mono- or di-substituted with an alkyl group” means that the alkyl may but need not be present, and the description includes situations where the heterocyclo group is mono- or disubstituted with an alkyl group and situations where the heterocyclo group is not substituted with the alkyl group.

“Alkylthio”, arylthio, and “heteroarylthio” respectively mean an alkyl group, aryl group, or heteroaryl group attached via a thioether linkage.

“Alkylsulfinyl”, “arylsulfinyl”, and “heteroarylsulfinyl” respectively mean an alkyl group, aryl group, or heteroaryl group attached via a sulfinyl linkage.

“Alkylsulfonyl”, “arylsulfonyl”, and “heteroarylsulfonyl” respectively mean an alkyl, aryl, or heteroaryl group attached via a sulfonyl linkage.

“Alkylsulfoxide”, “arylsulfoxide”, and “heteroarylsulfoxide” respectively mean an alkyl group, aryl group, or heteroaryl group attached via a sulfoxide linkage.

“Alkylcarbonyl”, “alkenylcarbonyl”, “arylcarbonyl”, “heteroarylcarbonyl”, and “aralkylcarbonyl” means an alkyl, alkenyl, aryl, heteroaryl, or aralkyl radical, as defined above, attached to a carbonyl radical. Examples of such radicals include substituted or unsubstituted methylcarbonyl, ethylcarbonyl, phenylcarbonyl and benzylcarbonyl.

“Heteroarylcarbonyl”, and “aralkylcarbonyl” means an alkyl, alkenyl, aryl, heteroaryl, or aralkyl radical, as defined above, attached to a carbonyl radical. Examples of such radicals include substituted or unsubstituted methylcarbonyl, ethylcarbonyl, phenylcarbonyl and benzylcarbonyl.

“Alkylcarbonyloxy”, “alkenylcarbonyloxy”, “arylcarbonyloxy”, refer to an alkyl, alkenyl, or aryl group attached to a CO₂ group, as in alkyl-CO₂—, alkenyl-CO₂—, aryl-CO₂—, respectively, where alkyl, alkenyl, and aryl are as defined herein. For example, alkylcarbonyloxy includes but is not limited to, acetoxy, ethylcarbonyloxy, n- or iso-propylcarbonyloxy, n-, iso-, sec- or tert-butylcarbonyloxy, n-pentylcarbonyloxy, n-hexylcarbonyloxy.

“Optionally substituted” means that the group at issue is optionally substituted independently with one, two or three substituents selected from alkyl, haloalkyl, halo, nitro, cyano, —OR (where R is hydrogen or alkyl), —NRR′ (where R and R′ are independently of each other hydrogen or alkyl), —COOR (where R is hydrogen or alkyl) or —CONR′R″ (where R′ and R″ are independently selected from hydrogen or alkyl), or as otherwise provided.

“Amino-protecting group” refers to those organic groups intended to protect nitrogen atoms against undesirable reactions during synthetic procedures e.g., benzyl, benzyloxycarbonyl (CBZ), t-butoxycarbonyl (BOC), trifluoroacetyl, and the like.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, if the Y and Y′ substituents in a compound of Formula (I) are attached to the same carbon are different, then the carbon to which they are attached is an asymmetric center and the compound of Formula (I) can exist as an (R)- or (S)-stereoisomer relative to that carbon. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counterion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.

A “pharmaceutically acceptable salt” of a compound means a salt that is pharmaceutically acceptable and that possesses the desired pharmacological activity of the parent compound. Such salts include:
1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or
2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Leaving group” has the meaning conventionally associated with it in synthetic organic chemistry i.e., an atom or group capable of being displaced by a nucleophile and includes halogen (such as chloro, bromo, iodo), alkanesulfonyloxy (such as mesyloxy or trifluorosulfonyloxy) or arenesulfonyloxy (such as tosyloxy), ester, or amino, and the like.

“Pro-drugs” means any compound which releases an active parent drug according to Formula (I) in vivo when such prodrug is administered to a mammalian subject. Prodrugs of a compound of Formula (I) are prepared by modifying functional groups present in the compound of Formula (I) in such a way that the modifications may be cleaved in vivo to release the parent compound. Prodrugs include compounds of Formula (I) wherein a hydroxy, sulfhydryl or amino group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of Formula (I), and the like.

“Treating” or “treatment” of a disease includes:
1) preventing the disease, i.e. causing the clinical symptoms of the disease not to develop in a mammal that may be exposed to or predisposed to the disease but does not yet experience or display symptoms of the disease;
2) inhibiting the disease, i.e., arresting or reducing the development of the disease or its clinical symptoms; or
3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

### Invention Compounds

Referring again to a compound of the invention, the following specific values are disclosed. Reference is first made to compounds of formula I and II.

A specific value for L is

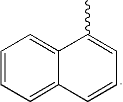

Other specific values for L include

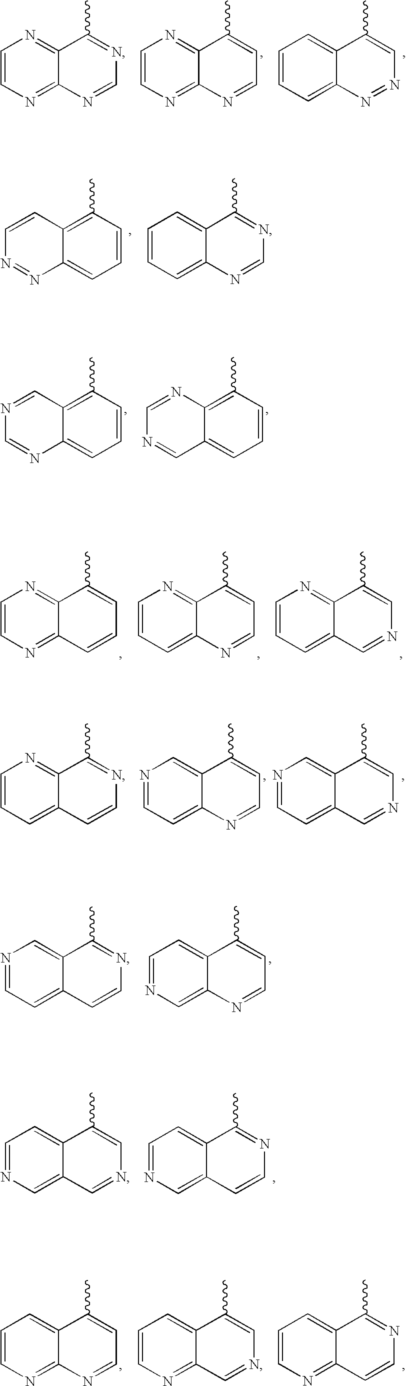

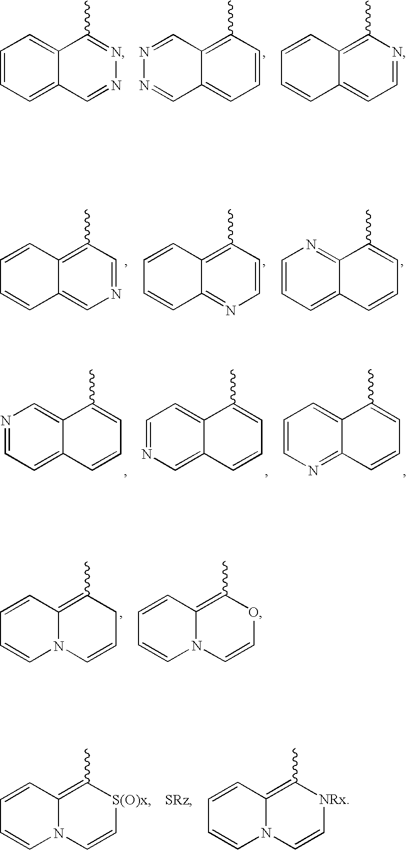

In the above values for L, [symbol] indicates the point of attachment. Also, each L may be optionally substituted with 1, 2, or 3 groups independently selected from the group consisting of H, halo, cyano, nitro, (C₁-C₆)alkanoyl, carboxy, (C₁-C₆)alkoxycarbonyl, (C₁-C₆)alkyl, hydroxyl, halo(C₁-C₆)alkyl, halo(C₁-C₆)alkoxy, (C₁-C₆)alkoxy, NHCO—(C₁-C₆)alkyl, SO₂(C₁-C₆)alkyl, SO₂NH(C₁-C₆)alkyl, or SO₂N((C₁-C₆)alkyl)₂. A specific value for a group that may be attached to L is H. Other specific values for groups that may be attached to L include cyano, methoxy.

Thus, in one embodiment, L is

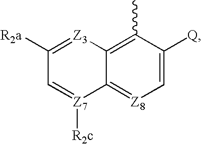

wherein

[symbol] indicates the point of attachment;

Z₃, Z₇, and Z₈ are C or N provided that when Z₇ is N, R_{2c} is absent;

Q is hydrogen, fluoro, or chloro; and

R₂ₐ is H, cyano, (C₁-C₆)alkyl, hydroxyl, halo, halo(C₁-C₆)alkoxy, (C₁-C₆)alkoxy.

More specifically, L is

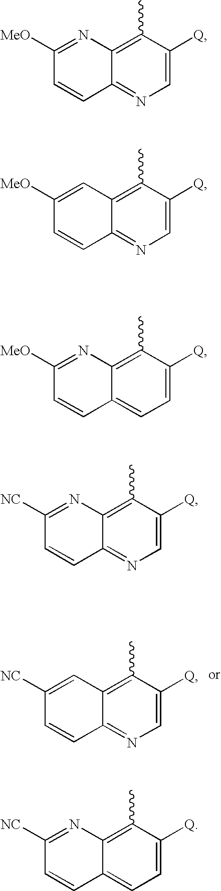

A specific value for X is NHCO. Other specific values for X include CO—CH₂, CH₂CH₂, O—CH₂, CHOHCH₂, or NHCH₂;

A specific value for

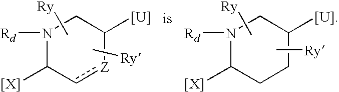

Specific values for Ry and Ry′ include H or (C₁-C₆)alkyl. Specific values for R_{d} include (C₁-C₆)alkyl, COMe, CO₂Me, CONH₂, CO₂H, —CH₂CH₂CO₂H, —CH₂CONH₂, —CH₂CO₂H, —CONHCH₃, SO₂Me, COCH₂OMe, COCH₂OH, and COCH₂OAc.

Another specific value for

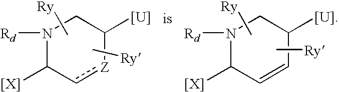

Specific values for Ry and Ry′ include H or (C₁-C₆)alkyl. Specific values for R_{d} include H, (C₁-C₆)alkyl, COMe, CO₂Me, CONH₂, CO₂H, —CH₂CH₂CO₂H, —CH₂CONH₂, —CH₂CO₂H, —CONHCH₃, SO₂Me, COCH₂OMe, COCH₂OH, and COCH₂OAc.

Another specific value for

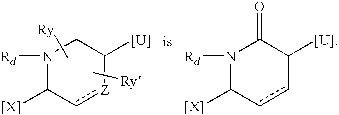

Another specific value for

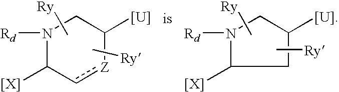

Specific values for Ry and Ry′ include H or (C₁-C₆)alkyl. Specific values for R_{d} include H, (C₁-C₆)alkyl, COMe, CO₂Me, CONH₂, CO₂H, —CH₂CH₂CO₂H, —CH₂CONH₂, —CH₂CO₂H, —CONHCH₃, SO₂Me, COCH₂OMe, COCH₂OH, and COCH₂OAc.

A specific value for U is CH₂. Other specific values for CH₂, CH₂CH₂, CH═CH, or C≡C. Each hydrogen may be optionally replaced by fluoro or (C₁-C₆)alkyl.

A specific value for R is R is benzo[1,2,5]thiadiazol-5-yl. Other specific values for R include 4H-benzo[1,4]thiazin-3-one-6-yl, 2,3-dihydro-benzo[1,4]dioxin-6-yl, benzo[1,2,3]thiadiazol-5-yl, 3-oxo-3,4-dihydro-2H-benzo[1,4]oxazin-6-yl, 7-fluoro-3-oxo-3,4-dihydro-2H-benzo[1,4]oxazin-6-yl, 2-oxo-2,3-dihydro-1H-pyrido[2,3-b][1,4]thiazin-7-yl, 2,3-dihydro-[1,4]dioxino[2,3-c]pyridin-7-yl, 3-oxo-3,4-dihydro-2H-pyrido[3,2-b][1,4]oxazin-6-yl, [1,2,3]thiadiazolo[5,4-b]pyridin-6-yl, 3-oxo-3,4-dihydro-2H-pyrido[3,2-b][1,4]thiazin-6-yl, 7-chloro-3-oxo-3,4-dihydro-2H-pyrido[3,2-b][1,4]thiazin-6-yl, 7-fluoro-3-oxo-3,4-dihydro-2H-pyrido[3,2-b][1,4]thiazin-6-yl, 2-thienylthio-methyl, or 2,5-difluorophenylvinyl.

Turning now to a compound of formula II-1, a specific group of compounds of the invention are compounds of formula II-1a

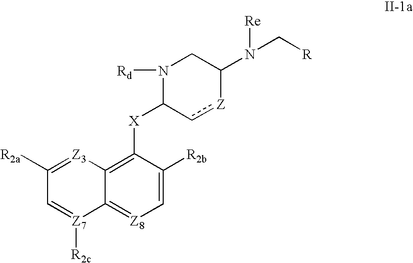

wherein Z₃, Z₇, Z₈ R_{2a-c}, R_{d}, Z, Rₑ, and R have the range of values described above.

Turning now to a compound of formula II-2, a specific group of compounds of the invention are compounds of formula II-2a

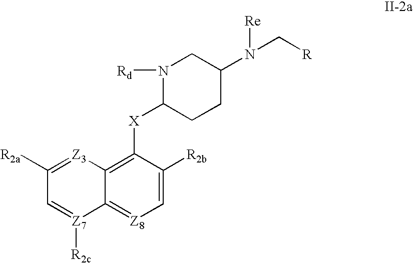

wherein Z₃, Z₇, Z₈ R_{2a-c}, R_{d}, Z, R_{d}, and R have the range of values described above.

Turning now to a compound of formula II-3, a specific group of compounds of the invention are compounds of formula II-3a

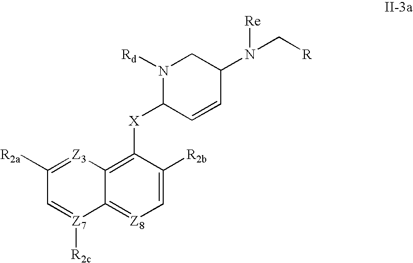

wherein Z₃, Z₇, Z₈ R^{2a-c}, R_{d}, Z, Rₑ, and R have the range of values described above.

Turning now to a compound of formula II-4, a specific group of compounds of the invention are compounds of formula II-4-a

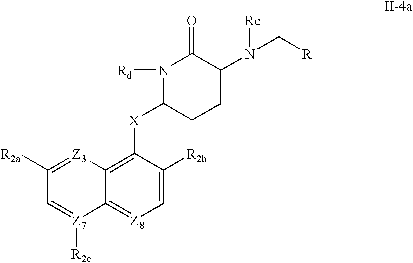

Turning now to a compound of formula II-5, a specific group of compounds of the invention are compounds of formula II-5a

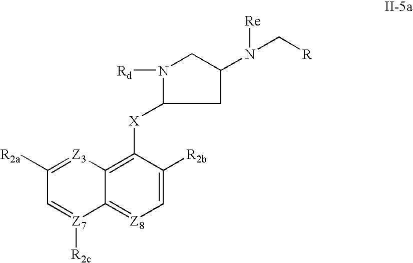

In one embodiment, the invention compounds of formula I, II, II-1, II-2, II-3, II-4, and II-5 have trans relative stereochemistry as depicted in compounds of formulas II-1A or I-1B.

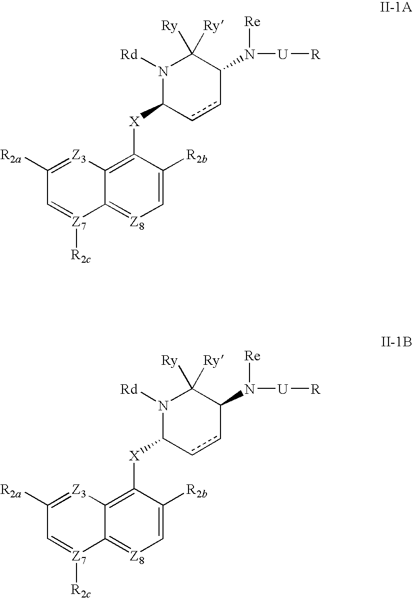

### Preparation of Invention Compounds

In a further aspect, the present invention provides a process for preparing a compound of the invention or a pharmaceutically-acceptable salt or an in-vivo hydrolysable ester thereof. It will be appreciated that during certain of the following processes, certain substituents may require protection to prevent their undesired reaction. The skilled chemist will appreciate when such protection is required, and how such protecting groups may be put in place and later removed.

Examples of protecting groups are disclosed in, for example, ‘Protective Groups in Organic Synthesis’ by Theodora Green (John Wiley & Sons, 1999). Protecting groups may be removed by any convenient method described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group that may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience, 2001), Jerry March or Houben-Weyl, Methoden der Organischen Chemie). The preparation of such starting materials is described within the accompanying non-limiting Examples. Alternatively, necessary starting materials are obtainable by analogous procedures to those illustrated that are within the ordinary skill of an organic chemist. Information on the preparation of necessary starting materials or related compounds (which may be adapted to form necessary starting materials) may also be found in the certain Patent Application Publications, the contents of the relevant process sections of which are incorporated herein by reference; for example WO2004/058144; US2004/0224946; WO2004/002992.

The skilled organic chemist will be able to use and adapt the information contained and referenced within the above references, and accompanying Examples therein and also the Examples herein, to obtain necessary starting materials, and products.

Thus, the present invention also provides that the compounds of the invention and pharmaceutically-acceptable salts and in vivo hydrolysable esters thereof, can be prepared by a process (a) to (h); and thereafter if necessary:

i) removing any protecting groups;

ii) forming a pro-drug (for example an in-vivo hydrolysable ester); and/or

iii) forming a pharmaceutically-acceptable salt;

wherein said processes (a) to (h) are as follows (wherein the variables are as defined above unless otherwise stated):
a) by modifying a substituent in, or introducing a substituent into another compound of the invention by using standard chemistry (see for example, Comprehensive Organic Functional Group Transformations (Pergamon, 1998), Katritzky, Meth-Cohn & Rees);

For example, a hydroxy group may be converted into a fluoro group; into an acyloxy group, for instance an acetoxy group; an amino group; a heterocyclyl group linked through nitrogen (optionally substituted on a carbon other than a carbon atom adjacent to the linking nitrogen ring atom), for instance an optionally substituted amino group; such conversions of the hydroxy group taking place directly (for instance by acylation or Mitsunobu reaction) or through the intermediacy of one or more derivatives (for instance a mesylate or an azide); an acyloxy group may be converted into a hydroxy group or into the groups that may be obtained from a hydroxy group (either directly or through the intermediacy of a hydroxy group); an alkyl halide group may be converted to a hydroxyl group, an amino group, a thioalkyl group or a heterocyclyl group linked through nitrogen; a keto group may be reduced to a hydroxyl group or an saturated alkyl group.

b) As depicted in Scheme 1, by reaction of a compound of formula (II) (wherein X is a leaving group useful in palladium [II] coupling, for example chloride, bromide, iodide, or trifluoromethylsulfonyloxy) with an alkyl boronic acid derived in-situ from an olefin III and a borane such as 9-BBN or a similar borane that is known to the skilled artisan (such methods are well known, see for instance S. R. Chemler et al, Angew. Chem. Int. Ed. 2001, 40, 4544-4568), followed by deprotection and reductive ammination with an aldehyde. A protecting group (“PG”) may be necessary when Y is N in compound III. Attachment of U—R occurs via removal of the BOC group in IV, followed by reductive amination using the aldehyde shown. An extra deprotection step is required at the end of the synthesis (for suitable protection groups see: ‘Protective Groups in Organic Synthesis’ by Theodora Green (publisher: John Wiley & Sons, 1999).

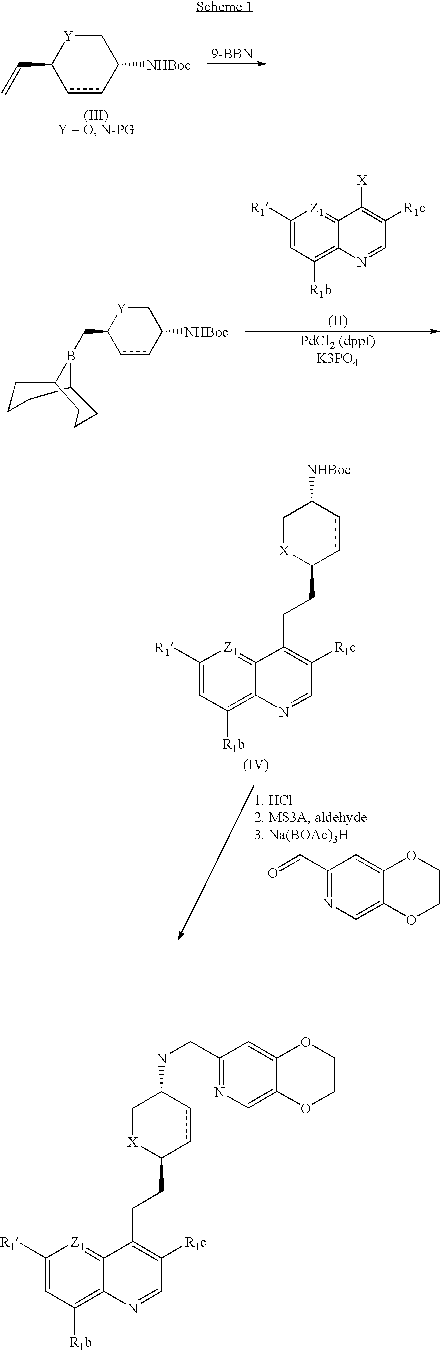

c) As depicted in Scheme 2, by reaction of a compound of formula (II) (wherein X is a leaving group useful in palladium [0 or II] coupling, for example chloride, bromide, iodide, or trifluoromethylsulfonyloxy) with an olefin (III), followed by hydrogenation to arrive at the same intermediate (IV) as under b) (such reactions are known as the “Heck” reaction: for a similar reaction see: S. Adam, Tetrahedron, 50 (11), 3327-3332, 1994).

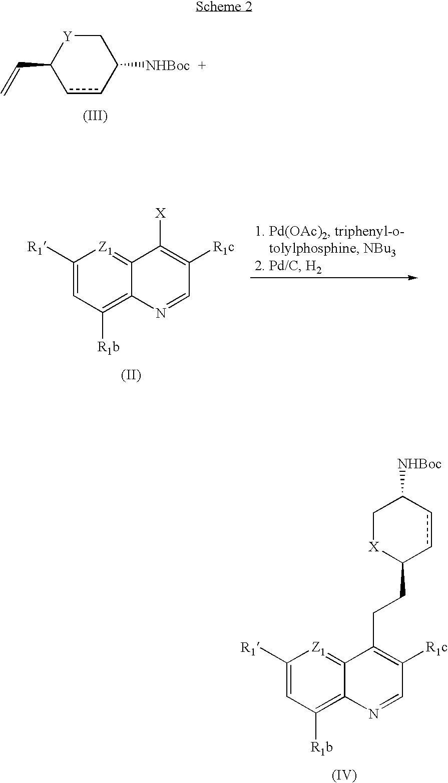

d) As depicted in Scheme 3, by reaction of a compound of formula (II) (wherein X is a leaving group useful in palladium [0 or II] coupling, for example chloride, bromide, iodide, or trifluoromethylsulfonyloxy) with a primary amide (V) (procedures for such reactions have been reported by Buchwald, e.g.: J. Yin, L. Buchwald, Organic Letters 2, No 8, 1101-1104, 2000), followed by deprotection and reductive ammination with an aldehyde. A protecting group (“PG”) may be necessary when Y is N in compound V. As a result, an extra deprotection step will be required at the end of the synthesis (for suitable protection groups see: ‘Protective Groups in Organic Synthesis’ by Theodora Green (publisher: John Wiley & Sons, 1999).

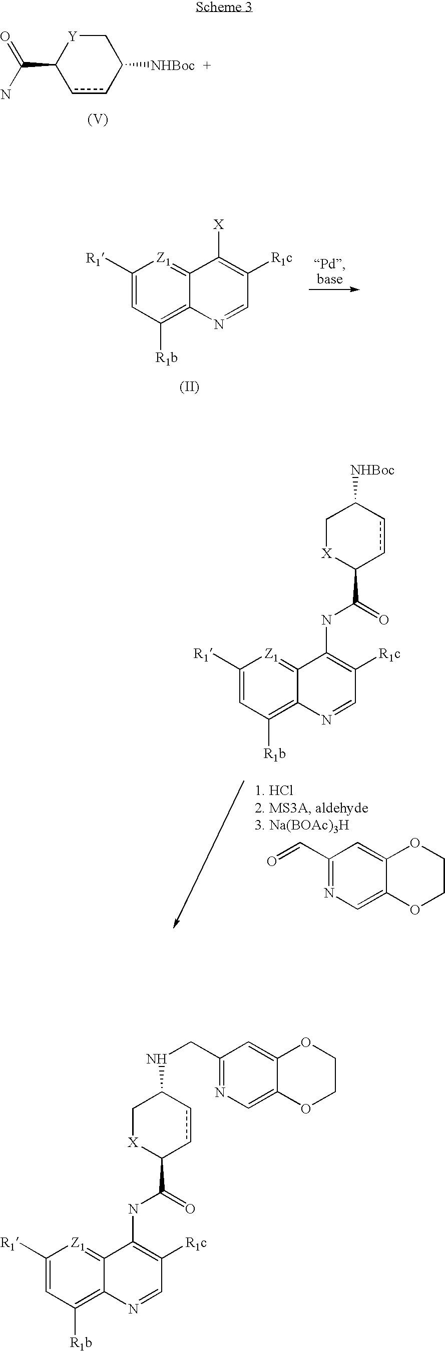

e) As depicted in Scheme 4, by reaction of a compound of formula (VI) with an alcohol, e.g. (VII) under Mitsunobu conditions, followed by deprotection and reductive ammination with an aldehyde. A protecting group (“PG”) may be necessary when Y is N in compound VII. As a result, an extra deprotection step will be required at the end of the synthesis (for suitable protection groups see: ‘Protective Groups in Organic Synthesis’ by Theodora Green (publisher: John Wiley & Sons, 1999).

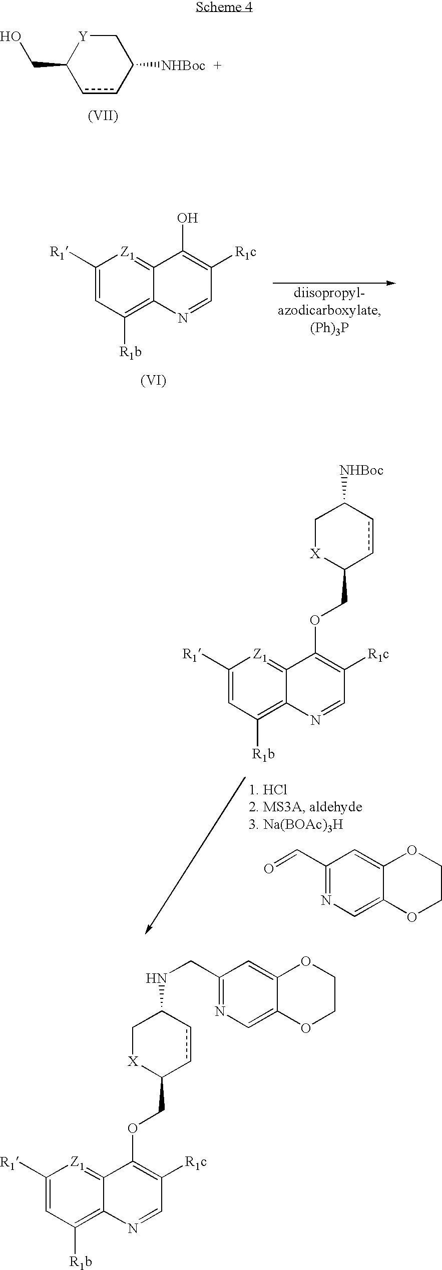

f) As depicted in Scheme 5, the precursor compounds (II), (V) and (VII) can be obtained for Y=O from the known precursors (VIII), (IX) and (X) (N. M. A. J. Kriek et al, Eur. J. Org. Chem. 2003, 2418-2427) by standard functional group manipulation and Wittig olefination.

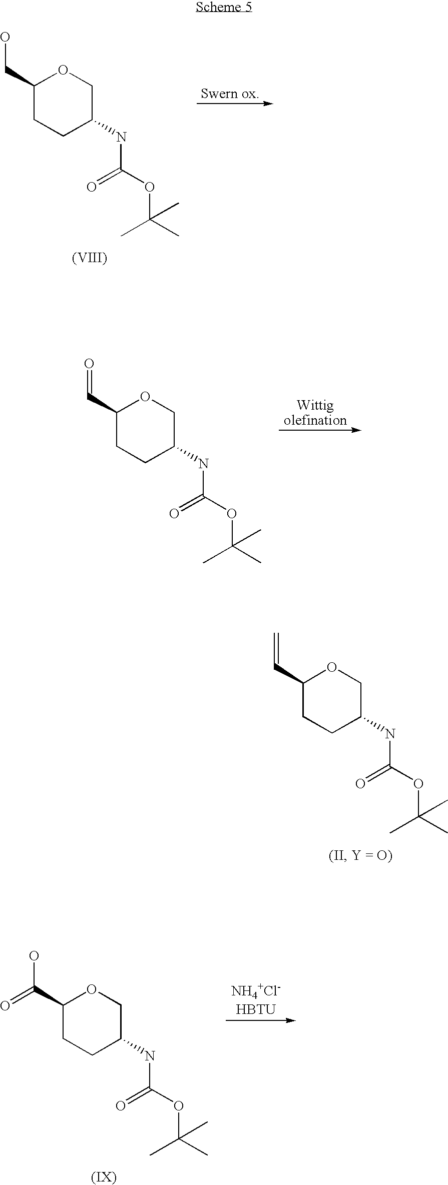

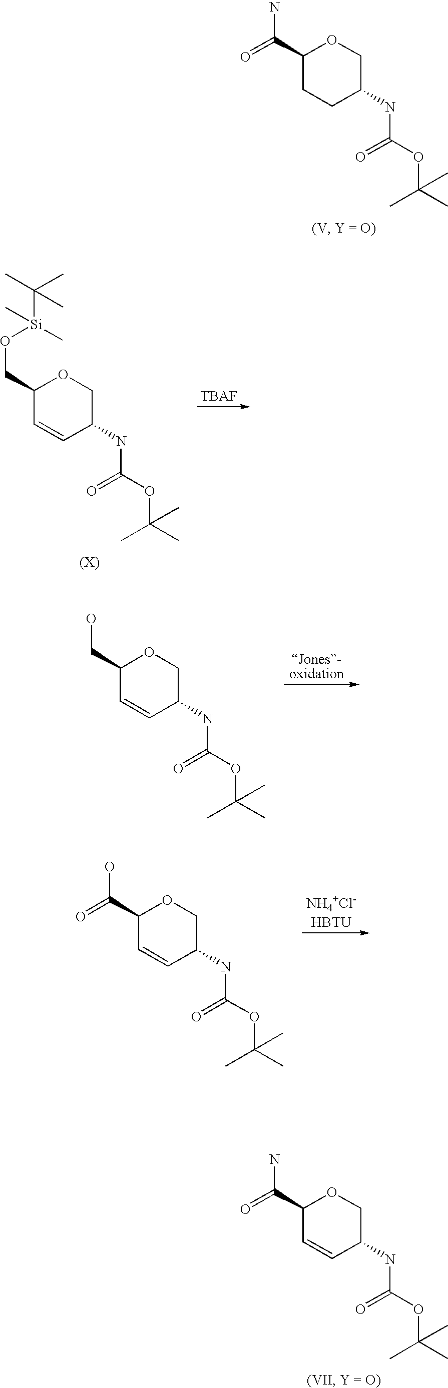

g) As depicted in Scheme 6, the precursor compounds (II) and (VII) can be obtained for Y=N from the known precursors pyroglutamic acid (Letavic et al. Bioorg. Med. Chem. Lett 12 (2002) 1387-1390) by standard functional group manipulation and Wittig olefination.

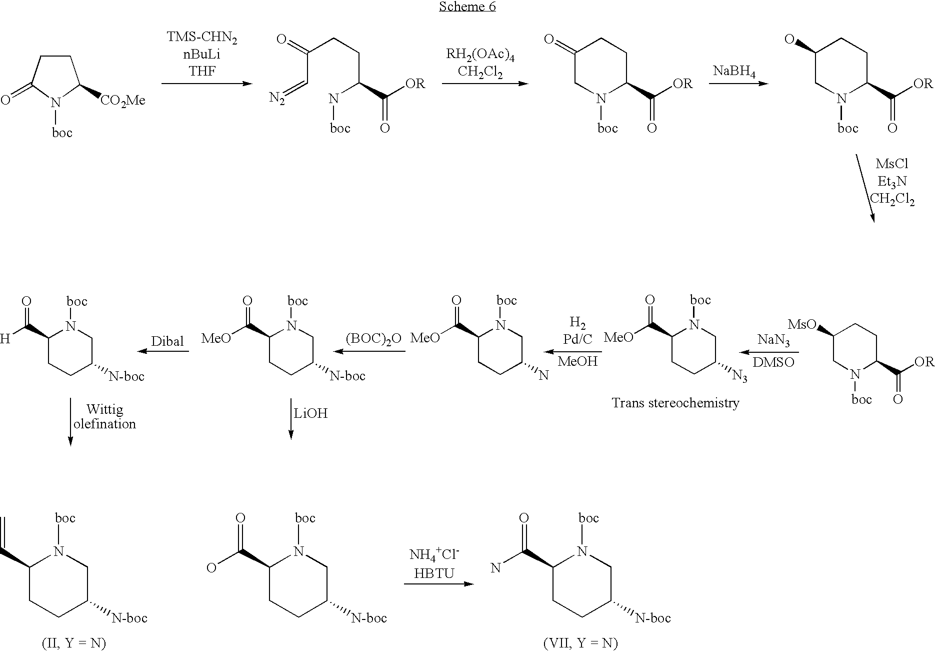

h) As depicted in Scheme 7, the invention compounds can be prepared by amide formation between an aniline compound (XI) with an acid (XII) (or an activated form of the acid, such as an acyl halide or an activated ester such as succinimidyl ester) under peptide coupling conditions, followed by reductive ammination. A protecting group (“PG”) may be necessary when Y is N in compound XII. As a result, an extra deprotection step will be required at the end of the synthesis (for suitable protection groups see: ‘Protective Groups in Organic Synthesis’ by Theodora Green (publisher: John Wiley & Sons, 1999).

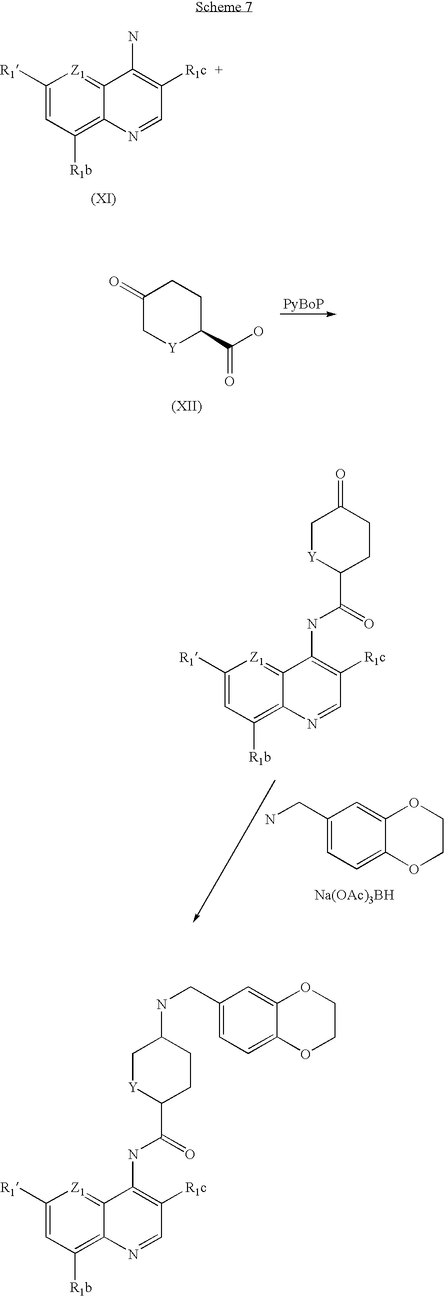

With respect to (a)-(h) and Schemes 1-7 above, the removal of any protecting groups, the formation of pharmaceutically-acceptable salts and/or the formation of in-vivo hydrolysable esters or amides are within the skill of an ordinary organic chemist using standard techniques. Furthermore, details regarding these transformations, for example the preparation of in-vivo hydrolysable ester prodrugs has been provided in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

Compounds of the formula (II) wherein X is a triflate or a bromide derivative and compounds of the formula (VI) may be made according to the processes described in WO 04/058144. Compounds of the formula (XI) can be obtained from the triflates of formula (II), by treatment with n-propylamine/HCl (Bioorg. Med. Chem. Lett, 11, 1907-1910, 2001).

### Biological Activity

The ability of the invention compounds disclosed herein to achieve an antibacterial effect is demonstrated by the following tests.

Enzyme Potency Testing Methods

Supercoiling Assay Description:

Compounds were tested for inhibition of Escherichia coli DNA supercoiling activity as follows. Assays were performed in polypropylene multiwell plates in 50 μl reactions containing 35 mM Tris-HCl (pH 7.5), 24 mM KCl, 4 mM MgCl₂, 2 mM dithiothreitol, 1.8 mM spermidine, 5% (v/v) glycerol, 200 nM bovine serum albumin, 1.25% (v/v) DMSO, 3 mM ATP, 10 ng/ml relaxed pBR322 plasmid, 0.6 nM DNA gyrase, and test compound. Reactions were quenched after 1 hour by the addition of 10 μl of 30% (w/v) Ficoll-400, 10 mM EDTA, and 5% sodium dodecyl sulfate. Twenty-five μl of each sample was loaded onto a 0.8% (w/v) agarose gel and electrophoresed. The gel and gel buffer contained 1×TBE buffer (89 mM Tris base, 89 mM boric acid, and 2 mM EDTA at pH 8.3). After electrophoresis for 3 hours at 70V, the gel was stained with ethidium bromide and visualized by excitation with ultraviolet light. The fluorescence intensity of the most supercoiled plasmid band was used to measure gyrase activity. Compound potency was based IC₅₀ measurements determined from reactions performed with eight 2-fold serial dilutions of each compound and a control without compound.

Compounds of the Examples generally have an IC₅₀ of <20 μg/ml.

ATPase Assay Description:

Compounds were tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays were performed in multiwell plates in 100 μl reactions containing: 50 mM TRIS buffer pH 7.5, 75 mM ammonium acetate, 5.5 mM magnesium chloride, 0.5 mM ethylenediaminetetraacetic acid, 5% glycerol, 1 mM 1,4-dithio-DL-threitol, 200 nM bovine serum albumin, 16 μg/ml sheared salmon sperm DNA, 4 nM E. coli GyrA, 4 nM E. coli GyrB, 250 μM ATP, and compound in dimethylsulfoxide. Reactions were quenched with 150 μL of ammonium molybdate/malachite green detection reagent containing 1.2 mM malachite green hydrochloride, 8.5 mM ammonium molybdate tetrahydrate, and 1 M hydrochloric acid. Plates were read in an absorbance plate reader at 625 μm and percent inhibition values were calculated using dimethylsulfoxide (2%)-containing reactions as 0% inhibition and novobiocin-containing (2 μM) reactions as 100% inhibition controls. Compound potency was based on IC₅₀ measurements determined from reactions performed in the presence of 10 different compound concentrations.

Compounds of the Examples generally have an IC₅₀ of <20 μg/ml.

Bacterial Susceptibility Testing Methods

Compounds were tested for antimicrobial activity by susceptibility testing in liquid media in a 96 well format. Compounds were dissolved in dimethylsulfoxide and tested in 10 doubling dilutions in the susceptibility assays. The organisms used in the assay were grown overnight on suitable agar media and then suspended in a liquid medium appropriate for the growth of the organism. The suspension was a 0.5 McFarland and a further 1 in 10 dilution was made into the same liquid medium to prepare the final organism suspension in 100 μL. Plates were incubated under appropriate conditions at 37° C. for 24 hours prior to reading. The Minimum Inhibitory Concentration (MIC) was determined as the lowest drug concentration able to reduce growth by 80% or more.

Compounds were evaluated against a panel of Gram-positive species, including Staphylococcus aureus, Streptococcus pneumoniae, Streptococcus pyogenes, and Enterococcus faecium. In addition, compounds were evaluated against a panel of Gram-negative species including Haemophilus influenzae, Escherichia coli and Moraxella catarrhalis. Compounds of the present invention have MIC's less than or equal to 32 μg/ml versus all the organisms named above.

### Pharmaceutical Formulations

In another embodiment, the present invention provides a pharmaceutical composition which comprises a compound of formula (I) admixed with a pharmaceutically-acceptable carrier, diluent, or excipient.

The invention compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (i.e. through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents.

The compositions of the invention may be obtained by conventional procedures using conventional pharmaceutical diluents, carriers, or excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. A suitable pharmaceutical composition of this invention is one suitable for oral administration in unit dosage form. For example, a formulation intended for oral administration to humans will generally contain, for example, a therapeutically effective amount of active agent compounded with an appropriate and convenient amount of excipients which may vary from about 1 to about 98 percent by weight of the total composition. For further information on Routes of Administration and Dosage Regimes the reader is referred to Chapter 25.3 in Volume 5 of Comprehensive Medicinal Chemistry (Corwin Hansch; Chairman of Editorial Board), Pergainon Press 1990.

In another aspect a pharmaceutical composition of the invention is one suitable for intravenous, subcutaneous or intramuscular injection. Each patient may receive, for example, a daily intravenous, subcutaneous or intramuscular dose of a compound of this invention, the composition being administered 1 to 4 times per day. The intravenous, subcutaneous and intramuscular dose may be given by means of a bolus injection. Alternatively, the intravenous dose may be given by continuous infusion over a period of time. Alternatively, each patient may receive a daily oral dose which may be approximately equivalent to the daily parenteral dose, the composition being administered 1 to 4 times per day.

### EXAMPLES

The invention will now be illustrated by the following non-limiting examples.

### Example 1

### (2,3-Dihydro[1,4]dioxino[2,3-c]pyridin-7-ylmethyl){(3R,6R)-6-[2-(3-fluoro-6-methoxy-1,5-naphthyridin-4-yl)ethyl]tetrahydro-2H-pyran-3-yl}amine

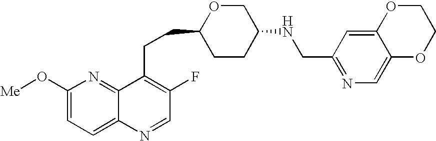

A solution of {(3R,6R)-6-[2-(3-fluoro-6-methoxy-1,5-naphthyridin-4-yl)ethyl]tetrahydro-2H-pyran-3-yl}amine (Intermediate 1, crude, approximately 0.089 mmol) and 2,3-dihydro[1,4]dioxino[2,3-c]pyridine-7-carbaldehyde (WO 2004/058144) (15 mg, 0.09 mmol) in dry chloroform/methanol (5 mL, 1:1) were heated over 3 Å molecular sieves at 70° C. for 3 hours. The reaction mixture was cooled to 0° C., sodium triacetoxy borohydride (58 mg, 0.272 mmol) was added and the resulting mixture was stirred at room temperature over night. The reaction mixture was acidified with concentrated HCl to pH of approximately 1, and was filtered and concentrated to dryness under reduced pressure. The residue was taken up in dichloromethane (20 mL) and saturated aqueous sodium hydrogen carbonate solution (5 mL), and the phases were separated. The aqueous phase was back extracted twice with dichloromethane (2×20 mL) and the combined organic phases were dried over anhydrous sodium sulfate. Chromatography on silica gel with dichloromethane/methanol (15:1), containing 0.125% ammonium hydroxide, gave the free base of the title compound as a slightly yellow film. The free base was taken up in dichloromethane (0.5 mL), isopropanol (2 mL) was added, followed by addition of 1M HCl in ether (50 μL). The resulting mixture was concentrated to dryness. Precipitation from dichloromethane (0.5 mL) with hexanes (2 mL) gave 14 mg (32% yield) of the hydrochloride salt of the product as a colorless solid.

¹H-NMR (DMSO-d₆): δ 1.20-4.50 (m, 18H); 4.03 (s, 3H); 7.12 (s, 1H); 7.23 (d, 1H); 8.19 (s, 1H); 8.27 (d, 1H); 8.77 (s, 1H); 9.14 (brs, 2H).

MS (ESP): 455.23 (MH⁺) for C₂₄H₂₇FN₄O₄

The intermediates for Example 1 were prepared as follows:

### Intermediate 1: {(3R,6R)-6-[2-(3-Fluoro-6-methoxy-1,5-naphthyridin-4-yl)ethyl]tetrahydro-2H-pyran-3-yl}amine

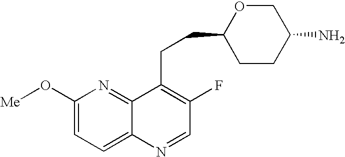

A solution of tert-butyl {(3R,6R)-6-[2-(3-fluoro-6-methoxy-1,5-naphthyridin-4-yl)ethyl]tetrahydro-2H-pyran-3-yl}carbamate (Intermediate 2, 36 mg, 0.089 mmol) in dioxane (2 mL) was treated at room temperature under vigorous stirring with a solution of HCl in dioxane (4M, 1 mL). After 6 hours, the reaction mixture was concentrated under reduced pressure and co-distilled once with isopropanol. The residue was taken up in dichloromethane (20 mL) and saturated aqueous sodium hydrogen carbonate solution (5 mL), and the phases were separated. The aqueous phase was extracted twice with dichloromethane (2×10 mL) and the combined organic phases were dried over anhydrous sodium sulfate to give 28 mg (100% yield) of the crude product as an oil.

MS (ESP): 306.09 (MH⁺) for C₁₆H₂₀FNO₄

### Intermediate 2: tert-Butyl {(3R,6R)-6-[2-(3-fluoro-6-methoxy-1,5-naphthyridin-4-yl)ethyl]tetrahydro-2H-pyran-3-yl}carbamate

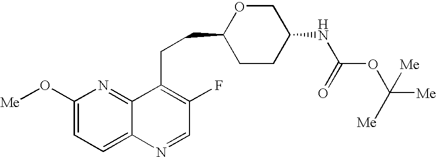

A solution of tert-butyl [(3R,6S)-6-vinyltetrahydro-2H-pyran-3-yl]carbamate (Intermediate 3, 40 mg, 0.176 mmol) in dry tetrahydrofuran (THF) (2 mL) was treated at 0° C. under stirring with a solution of 9-borabicyclo[3.3.1]nonane in hexanes (0.4M, 0.88 mL, 0.352 mmol). The reaction mixture was allowed to warm to room temperature and was stirred for 3 hours. A solution of potassium phosphate (3M, 0.123 mL) and 8-bromo-7-fluoro-2-methoxy-1,5-naphthyridine (WO 2004/058144, 50 mg, 0.194 mmol) was added. The resulting reaction mixture was degassed, flushed with nitrogen and then [1,1′-bis(diphenylphosphino)ferrocene]dichloropalladium (7 mg, 0.09 mmol, complex with dichloromethane) was added. The reaction mixture was stirred at room temperature over night and then at 50° C. for 3 hours. It was diluted with ethyl acetate (50 mL), washed with water (2×50 mL) and dried over anhydrous sodium sulfate. Chromatography on silica gel with hexanes/ethyl acetate (5:2) gave 36 mg (50% yield) of the product as a colorless solid.

¹H-NMR (benzene) δ: 0.73 (m, 1H); 1.12-1.80 (m, 4H); 1.45 (s, 9H); 1.93 (m, 2H); 2.68 (dd, 1H); 2.97 (m, 1H); 3.37 (m, 1H); 3.65-3.95 (m, 2H); 3.78 (s, 3H); 4.15 (m, 1H); 6.70 (d, 1H); 8.01 (d, 1H); 8.56 (s, 1H).

MS (ESP): 405.99 (MH⁺) for C₂₁H₂₈FN₃O₄

### Intermediate 3: tert-Butyl [(3R,6S)-6-vinyltetrahydro-2H-pyran-3-yl]carbamate

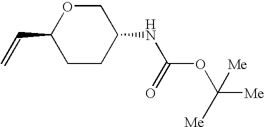

A solution of tert-butyl [(3R,6S)-6-formyltetrahydro-2H-pyran-3-yl]carbamate (Intermediate 4, 310 mg, 1.35 mmol) in toluene/THF (15 mL, 3:1) was treated at −78° C. under stirring with bis(cyclopentadienyl)-μ-chloro(dimethylaluminum)-β-methylenetitanium (Tebbe's reagent) (0.5M in toluene, 3.5 mL, 1.76 mmol). The reaction mixture was allowed to warm to room temperature over 3 hours. Aqueous sodium hydroxide solution (15%, 3.5 mL) was added slowly. The reaction mixture was then diluted with water (100 mL) and extracted with ethyl acetate (3×200 mL). The combined organic phases were washed with brine (2×150 mL) and dried over anhydrous sodium sulfate. Chromatography on silica gel with hexanes/ethyl acetate (7:1) gave 42 mg (13% yield) of product as a colorless solid.

¹H-NMR (DMSO-d₆) δ: 1.30 (m, 1H); 1.38 (s, 9H); 1.44 (m, 1H); 1.72 (m, 1H); 1.85 (m, 1H); 2.99 (dd, 1H); 3.30-3.40 (m, 1H, under HDO); 3.67 (m, 1H); 3.81 (m, 1H); 5.04 (m, 1H); 5.18 (m, 1H); 5.81 (m, 1H); 6.78 (d, 1H).

MS (ESP): 250.23 (MNa⁺) for C₁₂H₂₁NO₃

### Intermediate 4: tert-Butyl [(3R,6S)-6-formyltetrahydro-2H-pyran-3-yl]carbamate

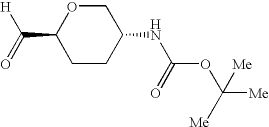

To a solution of oxalyl chloride (0.216 mL, 2.47 mmol) in dichloromethane (5 mL) at −50° C. was drop wise added a solution of dimethylsulfoxide (DMSO) (0.216 mL) in dichloromethane (5 mL). The mixture was stirred for 20 minutes, then a solution of tert-butyl [(3R,6S)-6-(hydroxymethyl)tetrahydro-2H-pyran-3-yl]carbamate (Intermediate 5, 440 mg, 1.9 mmol) in dichloromethane (5 mL) was added dropwise and the resulting mixture was stirred for one hour at −50° C. A solution of diisopropylethyl amine (1.6 mL, 9.5 mmol) in dichloromethane (4 mL) was added dropwise, and the reaction mixture was allowed to warm to −40° C. and was stirred for 1 hour. The temperature of the reaction mixture was then allowed to reach room temperature over 2 hours. The solvent was evaporated under reduced pressure, and the residue was co-distilled once with toluene and then chromatographed on silica gel with hexanes/ethyl acetate (1:1) to give 310 mg (71% yield) of product as a colorless solid.

¹H-NMR (DMSO-d₆) δ: 1.31-1.80 (m, 2H); 1.38 (s, 9H); 1.85-1.98 (m, 2H); 3.06 (dd, 1H); 3.30-3.40 (m, 1H, under HDO); 3.78 (dd, 1H); 3.89 (m, 1H); 6.85 (d, 1H); 9.53 (s, 1H).

### Intermediate 5: tert-Butyl [(3R,6S)-6-(hydroxymethyl)tetrahydro-2H-pyran-3-yl]carbamate

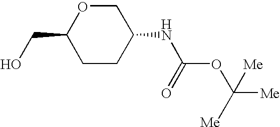

tert-Butyl [(3R,6S)-6-(hydroxymethyl)-3,6-dihydro-2H-pyran-3-yl]carbamate (Intermediate 6, 0.6 g, 2.62 mmol) was hydrogenated in ethyl acetate (10 mL) at room temperature and normal pressure over palladium/charcoal (10%, wet) for 5 hours. The reaction mixture was filtered through a 0.45 μm membrane and solvent was evaporated under reduced pressure. Chromatography on silica gel with ethyl acetate/hexanes (1:1 to 2:1) gave 0.486 g (80% yield) of the product as a colorless solid.

¹H-NMR (DMSO-d₆) δ: 1.21 (m, 1H); 1.30-1.42 (m, 1H); 1.38 (s, 9H); 1.64 (m, 1H); 1.84 (m, 1H); 2.92 (dd, 1H); 3.14 (m, 1H); 3.25-3.38 (m, 3H, under HDO); 3.78 (m, 1H); 4.59 (dd, 1H); 6.74 (d, 1H).

MS (ESP): 254(MNa⁺) for C₁₁H₂₁NO₃

### Intermediate 6: tert-Butyl [(3R,6S)-6-(hydroxymethyl)-3,6-dihydro-2H-pyran-3-yl]carbamate

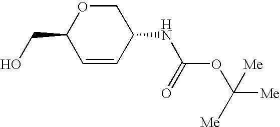

A solution of tert-butyl [(3R,6S)-6-({[tert-butyl(dimethyl)silyl]oxy}methyl)-3,6-dihydro-2H-pyran-3-yl]carbamate (N. M. A. J. Kriek et al, Eur. J. Org. Chem. 2003, 2418-2427) (17 g, 49.5 mmol) in dry THF (200 mL) was cooled to 0° C. and treated dropwise with a solution of tetrabutylammonium fluoride in THF (1M, 60 mL, 60 mmol). The mixture was stirred for 30 minutes at 0° C. Potassium phosphate buffer (pH 7, 1M, 200 mL) was added, and the THF was removed under reduced pressure. The aqueous mixture was extracted with ethyl acetate and the organic phase was dried over anhydrous sodium sulfate and evaporated to dryness under reduced pressure. The residue was chromatographed on silica gel with hexanes/acetone (5:2) to give 10 g (89% yield) of the product as a colorless solid.

¹H-NMR (CDCl₃) δ: 1.43 (s, 9H); 3.40 (dd, 1H); 3.56-3.67 (m, 2H); 4.09 (dd, 1H); 4.14-4.28 (m, 2H); 4.57 (m, 1H); 5.74 (m, 1H); 5.87 (m, 1H) (one proton, either the OH or the NH were not observed).

### Example 2

### (2S,5R)-5-[(2,3-Dihydro[1,4]dioxino[2,3-c]pyridin-7-ylmethyl)amino]-N-(6-methoxy-1,5-naphthyridin-4-yl)tetrahydro-2H-pyran-2-carboxamide

(2S,5R)-5-Amino-N-(6-methoxy-1,5-naphthyridin-4-yl)tetrahydro-2H-pyran-2-carboxamide (Intermediate 7, 60 mg, 0.2 mmol) was reacted with 2,3-dihydro[1,4]dioxino[2,3-c]pyridine-7-carbaldehyde (WO 2004/058144) (36 mg, 0.22 mmol) and sodium triacetoxy borohydride (126 mg, 0.60 mmol) following the procedure described under Example 1 to give 51 mg (53% yield) of the hydrochloride salt of the product as a colorless solid, mp>230° C. (decomposed).

¹H-NMR (DMSO-d₆) δ: 1.65 (m, 1H); 1.86 (m, 1H); 2.26 (m, 1H); 2.35 (m, 1H); 3.29 (m, 1H); 3.78 (dd, 1H); 4.12 (s, 3H); 4.24-4.48 (m, 7H); 4.52 (m, 1H); 7.41 (s, 1H); 7.61 (d, 1H); 8.31 (s, 1H); 8.57 (d, 1H); 8.62 (d, 1H); 8.95 (d, 1H); 9.88 (brs, 2H); 10.72 (s, 1H).

MS (ESP): 452.10 (MH⁺) for C₂₃H₂₅N₅O₅

The intermediates for Example 2 were prepared as follows:

### Intermediate 7: (2S,5R)-5-Amino-N-(6-methoxy-1,5-naphthyridin-4-yl)tetrahydro-2H-pyran-2-carboxamide

tert-Butyl ((3R,6S)-6-{[(6-methoxy-1,5-naphthyridin-4-yl)amino]carbonyl}tetrahydro-2H-pyran-3-yl)carbamate (Intermediate 8, 228 mg, 0.567 mmol) was deprotected and converted into the free base using the procedure described under Intermediate 1 to give 171 mg (100% yield) of the product as a colourless oil.

MS (ESP): 303.20 (MH⁺) for C₁₆H₁₈N₄O₃

### Intermediate 8: tert-Butyl ((3R,6S)-6-{[(6-methoxy-1,5-naphthyridin-4-yl)amino]carbonyl}tetrahydro-2H-pyran-3-yl)carbamate

tert-Butyl [(3R,6S)-6-(aminocarbonyl)tetrahydro-2H-pyran-3-yl]carbamate (Intermediate 9, 200 mg, 0.82 mmol), tris(dibenzylideneacetone) dipalladium (0) (15 mg, 0.016 mmol), cesium carbonate (333 mg, 1.02 mmol) and 2,2′-bis(diphenylphosphino)-1,1′-binaphthyl (31 mg, 0.05 mmol) were mixed under nitrogen in dry dioxane (6 mL). 6-Methoxy-1,5-naphthyridin-4-yl trifluoromethanesulfonate (WO 2002008224) (252 mg, 0.82 mmol) was added and the mixture was heated at 100° C. for 3 hours. The reaction mixture was cooled to room temperature, filtered through a 0.45 μm membrane and the solvent was removed under reduced pressure. Chromatography on silica gel with hexanes/acetone (2:1) gave 233 mg (71% yield) of the product as a colorless hard foam.

¹H-NMR (DMSO-d₆) δ: 1.38 (s, 9H); 1.55 (m, 2H); 1.96 (m, 1H); 2.14 (m, 1H); 3.24 (dd, 1H); 3.46 (m, 1H); 4.05 (s, 3H); 4.00-4.15 (m, 2H); 6.61 (d, 1H); 7.32 (d, 1H); 8.27 (d, 1H); 8.37 (d, 1H); 8.69 (d, 1H); 10.46 (s, 1H).

MS (ESP): 403.21 (MH⁺) for C₂₀H₂₆N₄O₅

### Intermediate 9: tert-Butyl [(3R,6S)-6-(aminocarbonyl)tetrahydro-2H-pyran-3-yl]carbamate

To a solution of (2S,5R)-5-[(tert-butoxycarbonyl)amino]tetrahydro-2H-pyran-2-carboxylic acid (N. M. A. J. Kriek et al, Eur. J. Org. Chem. 2003, 2418-2427) (775 mg, 3.16 mmol) in DMF (10 mL) was added N-methylmorpholine (1.4 mL, 12.64 mmol), followed by addition of O-(7-azabenzotriazol-1-yl)-N,N,N′,N′-tetramethyluronium hexafluorophosphate (2.4 g, 6.31 mmol) and ammonium chloride (338 mg, 6.32 mmol). The resulting reaction mixture was stirred for 1 hour at room temperature. The solvent was removed under reduced pressure, and the residue was taken up in ethyl acetate (100 mL) and washed with potassium phosphate buffer (pH7, 1M, 20 mL). The aqueous phase was backextraced once with ethyl acetate (70 mL) and the combined organic phases were dried over sodium sulfate. The solvent was evaporated under reduced pressure and the product was precipitated from dichloromethane (30 mL) with hexanes (30 mL) to give 647 mg (84% yield) of the product as a colorless solid.

¹H-NMR (DMSO-d₆) δ: 1.36 (s, 9H); 1.26-1.50 (m, 2H); 1.80-2.00 (m, 2H); 2.99 (dd, 1H); 3.37 (m, 1H); 3.54 (m, 1H); 3.86 (m, 1H); 6.81 (d, 1H); 7.06 and 7.14 (2×s, 2H).

MS (ESP): 267.29 (MNa⁺) for C₁₁H₂₀N₂O₄

### Example 3

### (2S,5R)—N-(3-Chloro-6-methoxyquinolin-4-yl)-5-[(2,3-dihydro [1,4]dioxino [2,3-c]pyridin-7-ylmethyl)amino]tetrahydro-2H-pyran-2-carboxamide

(2S,5R)-5-Amino-N-(3-chloro-6-methoxyquinolin-4-yl)tetrahydro-2H-pyran-2-carboxamide (Intermediate 10, 60 mg, 0.179 mmol) was reacted with 2,3-dihydro[1,4]dioxino[2,3-c]pyridine-7-carbaldehyde (WO 2004/058144) (33 mg, 0.197 mmol) and sodium triacetoxy borohydride (114 mg, 0.54 mmol) following the procedure described under Example 1 to give 32 mg (34% yield) of the hydrochloride salt of the product as a colorless solid, mp 220° C.

¹H-NMR (DMSO-d₆) δ: 1.72 (m, 1H); 1.84 (m, 1H); 2.22 (m, 1H); 2.34 (m, 1H); 3.31 (m, 1H); 3.66 (dd, 1H); 3.89 (s, 3H); 4.20-4.50 (m, 8H); 7.16 (d, 1H); 7.26 (s, 1H); 7.48 (d, 1H); 7.99 (d, 1H); 8.26 (s, 1H); 8.78 (s, 1H); 9.53 (brs, 2H); 10.25 (s, 1H).

MS (ESP): 485.07 (MH⁺) for C₂₄H₂₅ClN₄O₅

The intermediates for Example 3 were prepared as follows:

### Intermediate 10: (2S,5R)-5-Amino-N-(3-chloro-6-methoxyquinolin-4-yl)tetrahydro-2H-pyran-2-carboxamide

tert-Butyl ((3R,6S)-6-{[(3-chloro-6-methoxyquinolin-4-yl)amino]carbonyl}tetrahydro-2H-pyran-3-yl)carbamate (Intermediate 11, 208 mg, 0.477 mmol) was deprotected and converted into the free base using the procedure described for Intermediate 1 to give 160 mg (quant) of the product as a colorless oil.

MS (ESP): 336.15 (MH⁺) for C₁₆H₁₈ClN₃O₃

### Intermediate 11: tert-Butyl ((3R,6S)-6-{[(3-chloro-6-methoxyquinolin-4-yl)amino]carbonyl}tetrahydro-2H-pyran-3-yl)carbamate

tert-Butyl [(3R,6S)-6-(aminocarbonyl)tetrahydro-2H-pyran-3-yl]carbamate (Intermediate 9, 200 mg, 0.82 mmol), tris(dibenzylideneacetone) dipalladium (0) (15 mg, 0.016 mmol), cesium carbonate (333 mg, 1.02 mmol), 2,2′-bis(diphenylphosphino)-1,1′-binaphthyl (31 mg, 0.05 mmol) and 4-bromo-3-chloro-6-methoxyquinoline (WO 2002040474) (223 mg, 0.82 mmol) were reacted following the procedure described for Intermediate 8, except that heating occurred for 24 hours. Chromatography on silica gel with hexanes/acetone (5:2) gave 212 mg (59% yield) of the product as a colorless solid.

¹H-NMR (DMSO-d₆) δ: 1.39 (s, 9H); 1.53 (m, 1H); 1.70 (m, 1H); 1.98 (m, 1H); 2.10 (m, 1H); 3.19 (dd, 1H); 3.50 (m, 1H); 3.88 (s, 3H); 4.00 (m, 1H); 4.05 (m, 1H); 6.90 (d, 1H); 7.11 (d, 1H); 7.45 (d, 1H); 7.98 (d, 1H); 8.77 (s, 1H); 10.09 (s, 1H).

MS (ESP): 436.13 (MH⁺) for C₂₁H₂₆ClN₃O₅

### Example 4

### ((3R,6S)-6-{[(3-Chloro-6-methoxyquinolin-4-yl)oxy]methyl}tetrahydro-2H-pyran-3-yl)(2,3-dihydro[1,4]dioxino[2,3-c]pyridin-7-ylmethyl)amine

((3R,6S)-6-{[(3-Chloro-6-methoxyquinolin-4-yl)oxy]methyl}tetrahydro-2H-pyran-3-yl)amine (Intermediate 12, 105 mg, 0.325 mmol) was reacted with 2,3-dihydro[1,4]dioxino[2,3-c]pyridine-7-carbaldehyde (WO 2004/058144) (54 mg, 0.325 mmol) and sodium triacetoxy borohydride (207 mg, 0.976 mmol) following the procedure described for Example 1 to give 113 mg (60% yield) of the hydrochloride salt of the product as a colorless solid, mp 191° C.

¹H-NMR (DMSO-d₆) δ: 1.48 (m, 1H); 1.73 (m, 1H); 1.85 (m, 1H); 2.27 (m, 1H); 3.17 (m, 1H); 3.50 (dd, 1H); 3.76 (m, 1H); 3.92 (s, 3H); 4.15-4.65 (m, 9H); 7.27 (s, 1H); 7.46 (dd, 1H); 7.55 (d, 1H); 7.96 (d, 1H); 8.25 (s, 1H); 8.73 (s, 1H); 9.51 (brs, 2H).

MS (ESP): 472.09 (MH⁺) for C₂₄H₂₆ClN₃O₅

The intermediates for Example 4 were prepared as follows:

### Intermediate 12: ((3R,6S)-6-{[(3-Chloro-6-methoxyquinolin-4-yl)oxy]methyl}tetrahydro-2H-pyran-3-yl)amine

tert-Butyl ((3R,6S)-6-{[(3-chloro-6-methoxyquinolin-4-yl)oxy]methyl}tetrahydro-2H-pyran-3-yl)carbamate (Intermediate 13, 156 mg, 0.37 mmol) was deprotected and converted into the free base using the procedure described under Intermediate 1 to give 105 mg (88% yield) of the product as a colorless hard foam.

The crude amine was directly used in the next step without further characterization.

### Intermediate 13: tert-Butyl ((3R,6S)-6-{[(3-chloro-6-methoxyquinolin-4-yl)oxy]methyl}tetrahydro-2H-pyran-3-yl)carbamate

To a solution of 3-chloro-6-methoxyquinolin-4-ol (WO 2004058144) (250 mg, 1.19 mmol), tert-butyl [(3R,6S)-6-(hydroxymethyl)tetrahydro-2H-pyran-3-yl]carbamate (Intermediate 5, 276 mg, 1.19 mmol) and triphenylphosphine (375 mg, 1.42 mmol) in dry THF (5 mL) was added diisopropylazodicarboxylate (0.258 mL, 1.31 mmol). The resulting mixture was stirred at room temperature overnight. The solvent was removed under reduced pressure and the residue was chromatographed on silica gel with hexanes/ethyl acetate (3:1 to 2:1) and then with dichloromethane/ethyl acetate (15:1 to 5:1) gave 159 mg (32% yield) of the product as a colorless solid, mp 173° C.

¹H-NMR (DMSO-d₆) δ: 1.37 (s, 9H); 1.35-1.50 (m, 2H); 1.72 (m, 1H); 1.91 (m, 1H); 3.03 (dd, 1H); 3.31 (m, 1H); 3.63 (m, 1H); 3.85 (m, 1H); 3.91 (s, 3H); 4.16 (dd, 1H); 4.26 (dd, 1H); 6.80 (d, 1H); 7.43 (dd, 1H); 7.58 (d, 1H); 7.93 (d, 1H); 8.68 (s, 1H).

### Example 5

### (2S,5R)-5-[(2,3-Dihydro[1,4]dioxino[2,3-c]pyridin-7-ylmethyl)amino]-N-(6-methoxy-1,5-naphthyridin-4-yl)piperidine-2-carboxamide

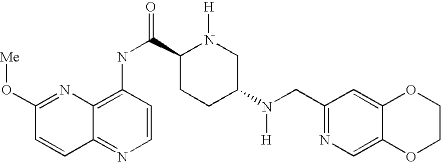

To a solution of tert-butyl (2S,5R)-5-[(2,3-dihydro[1,4]dioxino[2,3-c]pyridin-7-ylmethyl)amino]-2-{[(6-methoxy-1,5-naphthyridin-4-yl)amino]carbonyl}piperidine-1-carboxylate (0.20 g) (Intermediate 24) in dichloromethane (10 mL) was added trifluoracetic acid (1 mL). The resulting mixture was heated for two hours in a bath at 45° C., and then was concentrated under reduced pressure. The residue was purified by high pressure liquid chromatography (HPLC) on an Atlantic C18 5 μM column (A: 0.1% TFA/water; B: 0.1% TFA/acetonitrile) (0-95% B in 15 min gradient). Fractions containing product were collected, concentrated slightly, neutralized with solid Na₂CO₃ and extracted twice with ethyl acetate. The organic phase was washed with brine, dried over anhydrous sodium sulfate, filtered, and concentrated at reduced pressure to yield 59 mg of the tile compound.

¹H NMR (400 MHz, DMSO-D6) δ 1.25-1.32 (m, 1H) 1.41-1.51 (m, 1H) 1.87-1.96 (m, 1H) 1.97-2.05 (m, 1H) 2.35-2.45 (m, 1H) 3.09-3.19 (m, 2H) 3.68 (s, 2H) 4.04 (s, 3H) 4.25-4.35 (m, 4H) 6.95 (s, 1H) 7.30 (d, J=9.09 Hz, 1H) 8.00 (s, 1H) 8.25 (d, J=9.09 Hz, 1H) 8.36 (d, J=5.05 Hz, 1H) 8.66 (d, J=5.05 Hz, 1H) 11.05 (s, 1H)

ES (M+H)⁺=451.

The intermediates for Example 5 were prepared as follows:

### Intermediate 14: 1-tert-Butyl 2-methyl (2S)-5-oxopyrrolidine-1,2-dicarboxylate

To a solution of methyl (S)-(+)-2-pyrrolidone-5-carboxylate (10 g) in dichloromethane (100 mL) and triethylamine (15 mL) was added di-tert-butyl carbonate (19.5 g) and dimethylamino pyridine (DMAP) (0.5 g). After stirring overnight at room temperature, the reaction mixture was diluted with dichloromethane, washed with sodium hydrogen carbonate and brine, dried over anhydrous sodium sulfate, filtered and was concentrated. The residue was purified by flash chromatography using a FlashMaster™ and ethylacetate and hexane as eluants (Separtis, Germany) yielding 15.1 g of the title compound.

ES (M+Na)⁺=266.

### Intermediate 15: Methyl N-(tert-butoxycarbonyl)-6-diazonio-5-oxo-L-norleucinate

The title compound was prepared as provided in Bioorganic & Medicinal Chemistry Letters 12 (2002) 1387-1390. Thus, to a solution of trimethylsilyl-diazomethane (20 mL) in THF (75 mL) at −100° C. was added 1.6 M nBuLi in hexanes (25 mL). After 30 minutes at −100° C., the reaction mixture was added via canulla to a solution of 1-tert-butyl 2-methyl (2S)-5-oxopyrrolidine-1,2-dicarboxylate (7.57 g) in THF (300 mL) at −100° C. After stirring for 45 minutes at −100° C., the reaction was quenched with acetic acid/methanol (10 mL) and then was treated with saturated ammonium chloride solution and ethyl acetate. The organic phase was collected, washed with brine, dried over anhydrous sodium sulfate, filtered and concentrated to provide 9.28 g of the title compound.

ES (M+H)⁺=308.

### Intermediate 16: 1-tert-Butyl 2-methyl (2S)-5-oxopiperidine-1,2-dicarboxylate

The title compound was prepared as provided in Bioorganic & Medicinal Chemistry Letters 12 (2002) 1387-1390. Thus, to a refluxing solution of rhodium (II) acetate dimmer (Rh₂(OAc)₂) (130 mg) in benzene (400 mL) was added dropwise over an hour, a solution of methyl N-(tert-butoxycarbonyl)-6-diazonio-5-oxo-L-norleucinate (9.28 g) in benzene (50 mL). After 2 hours, the reaction was concentrated and purified by flash chromatography using a FlashMaster™ and ethylacetate and hexane as eluants to provide 3.7 g of the title compound.

GC-MS (M⁺)=257.

### Intermediate 17: 1-tert-Butyl 2-methyl (2S,5S)-5-hydroxypiperidine-1,2-dicarboxylate

To a solution of 1-tert-butyl 2-methyl (2S)-5-oxopiperidine-1,2-dicarboxylate (3.69 g) in methanol (100 mL) at 0° C. was added sodium borohydride (NaBH₄) (0.51 g). After 3 hours at 0° C., the reaction was concentrated slightly, diluted with ethyl acetate, washed with 1 M HCl, 1 M NaOH, and brine, dried over anhydrous sodium sulfate, filtered and concentrated to give 3.36 g of the title compound.

ES (M+Na)⁺=282.

### Intermediate 18: 1-tert-Butyl 2-methyl (2S,5S)-5-[(methylsulfonyl)oxy]piperidine-1,2-dicarboxylate

To a solution of 1-tert-butyl 2-methyl (2S,5S)-5-hydroxypiperidine-1,2-dicarboxylate (3.36 g) in dichloromethane (40 mL) were added triethylamine (2.8 mL) and methanesulfonyl chloride (1.2 mL). After 1½ hours, the reaction mixture was diluted with dichloromethane, washed with NaHCO₃ and brine, dried over anhydrous sodium sulfate, filtered and concentrated to give 4.54 g of the title compound.

### Intermediate 19: 1-tert-Butyl 2-methyl (2S,5R)-5-azidopiperidine-1,2-dicarboxylate

A solution of 1-tert-butyl 2-methyl (2S,5S)-5-[(methylsulfonyl)oxy]piperidine-1,2-dicarboxylate (0.62 g) and sodium azide (0.52 g) in dimethylformamide (DMF) (5 mL) was heated in a microwave vessel for 1 hour at 100° C. The reaction was diluted with ethyl acetate, washed with water, sodium hydrogen carbonate and brine, dried over anhydrous sodium sulfate, filtered and concentrated. The residue was purified by flash chromatography using a FlashMaster™ and ethylacetate and hexane as eluants to give 0.13 g of the title compound.

ES (M+H)⁺=307.

### Intermediate 20: 1-tert-Butyl 2-methyl (2S,5R)-5-aminopiperidine-1,2-dicarboxylate

A solution of 1-tert-butyl 2-methyl (2S,5R)-5-azidopiperidine-1,2-dicarboxylate (1.92 g) and 10% Pd/C (0.17 g) in methanol (20 μL) was stirred under hydrogen balloon. After 1½ hours, the reaction was filtered and concentrated to yield 1.60 g of the title compound.

ES (M+H)⁺=259.

### Intermediate 21: 1-tert-Butyl 2-methyl (2S,5R)-5-[(2,3-dihydro-1,4-benzodioxin-6-ylmethyl)amino]piperidine-1,2-dicarboxylate

To a solution of 1-tert-butyl 2-methyl (2S,5R)-5-aminopiperidine-1,2-dicarboxylate (1.60 g) in methanol (40 mL) were added 3 Å molecular sieves powder (0.17 g) and 2,3-dihydro[1,4]dioxino[2,3-c]pyridine-7-carbaldehyde (1.16 g). After heating at reflux for 2 hours, the reaction was cooled with an ice bath before adding NaCNBH₃ (0.47 g). After stirring overnight at room temperature, the reaction mixture was diluted with ethyl acetate, washed with water, sodium hydrogen carbonate and brine, dried over anhydrous sodium sulfate, filtered and concentrated. This residue was purified by flash chromatography using a FlashMaster™ with methylenechloride to methylenechloride/methanol (10:1) as eluants to give 1.26 g of the title compound.

ES (M+H)⁺=408.

### Intermediate 22: (2S,5R)-1-(tert-Butoxycarbonyl)-5-[(2,3-dihydro[1,4]dioxino[2,3-c]pyridin-7-ylmethyl)amino]piperidine-2-carboxylic acid

To a solution of 1-tert-butyl 2-methyl (2S,5R)-5-[(2,3-dihydro-1,4-benzodioxin-6-ylmethyl)amino]piperidine-1,2-dicarboxylate (0.53 g) in THF (5 mL), methanol (1 mL) and water (1 mL) was added solid lithium hydroxide (0.12 g). The resulting mixture was heated with a bath to a temperature of 45° C. After 3 hours, the reaction was concentrated to yield 0.69 g of the title compound.

ES (M+H)⁺=394.

### Intermediate 23: tert-Butyl (2S,5R)-2-(aminocarbonyl)-5-[(2,3-dihydro[1,4]dioxino[2,3-c]pyridin-7-ylmethyl)amino]piperidine-1-carboxylate

To a solution of (2S,5R)-1-(tert-butoxycarbonyl)-5-[(2,3-dihydro[1,4]dioxino[2,3-c]pyridin-7-ylmethyl)amino]piperidine-2-carboxylic acid (0.69 g) in DMF (5 mL) were added N-(3-dimethylaminopropyl)-N′-ethylcarbodiimide hydrochloride (EDAC) (0.23 g) and 1-hydroxy-7-azabenzotriazole (HOAT) (0.21 g). After 15 min, ammonium bicarbonate (0.38 g) was added. EDC (0.32 g), ammonium bicarbonate (0.40 g) and DMF (3 mL) were added after stirring overnight at room temperature. After 48 hours, the reaction was diluted with ethyl acetate and water. The aqueous layer was collected and extracted twice with ethyl acetate. The combined organic solution was dried over anhydrous sodium sulfate, filtered and concentrated. This residue was purified by flash chromatography using a FlashMaster™ with methylenechloride to methylenechloride/methanol (10:1) as eluants yielding 0.24 g of the title compound.

ES (M+H)⁺=393.

### Intermediate 24: tert-Butyl (2S,5R)-5-[(2,3-dihydro[1,4]dioxino[2,3-c]pyridin-7-ylmethyl)amino]-2-{[(6-methoxy-1,5-naphthyridin-4-yl)amino]carbonyl}piperidine-1-carboxylate

To a solution of tert-butyl (2S,5R)-2-(aminocarbonyl)-5-[(2,3-dihydro[1,4]dioxino[2,3-c]pyridin-7-ylmethyl)amino]piperidine-1-carboxylate (0.11 g) in dioxane (5 mL) were added tris(dibenzylideneacetone)dipalladium (0) Pd₂(dba)₃ (2 mg), racemic 2,2′-bis(diphenylphosphino)-1,1′-binaphthalene (BINAP) (20 mg), and cesium carbonate (CsCO₃) (0.10 g). The mixture was degassed by bubbling nitrogen gas into the solution before adding 6-methoxy-1,5-naphthyridin-4-yl trifluoromethanesulfonate (0.10 g). After 7 hours at bath temperature of 100° C., the reaction was cooled to room temperature and diluted with dichloromethane and water. The organic solution was collected, washed with brine, dried over anhydrous sodium sulfate, filtered and concentrated. This residue was purified by flash chromatography using a FlashMaster™ with dichloromethane to dichloromethane/methanol (10:1) as eluants to yield 0.20 g of the title compound.

ES (M+H)⁺=551.

### Example 6

### (2S,5S)-5-[(2,3-Dihydro-1,4-benzodioxin-6-ylmethyl)amino]-N-(6-methoxy-1,5-naphthyridin-4-yl)piperidine-2-carboxamide

To a solution of tert-butyl (2S,5S)-5-[(2,3-dihydro-1,4-benzodioxin-6-ylmethyl)amino]-2-{[(6-methoxy-1,5-naphthyridin-4-yl)amino]carbonyl}piperidine-1-carboxylate (0.31 g) (Intermediate 27) in dichloromethane (8 mL) was added TFA (2 mL). After 2 hours at a bath temperature of 45° C., the reaction was concentrated. The residue was purified by semi-prep HPLC on an Atlantic C18 5 μM column (A: 0.1% TFA/water; B: 0.1% TFA/acetonitrile) (0-95% B in 15 minutes gradient). Fractions containing the product were collected, concentrated slightly, neutralized with Na₂CO₃ (s) and extracted with twice with ethyl acetate. The organic solution was washed with brine, dried over anhydrous sodium sulfate, filtered and concentrated to yield 57 mg of the title compound.

¹H NMR (400 MHz, CHLOROFORM-D) δ ppm 1.62-1.68 (m, 1H) 1.78-1.90 (m, 2H) 2.22-2.33 (m, 1H) 2.65 (dq, J=7.04, 3.46 Hz, 1H) 2.82 (dd, J=13.26, 6.69 Hz, 1H) 3.03 (dd, J=13.39, 2.78 Hz, 1H) 3.56-3.64 (m, 1H) 3.68 (d, J=3.79 Hz, 2H) 4.02-4.10 (m, 3H) 4.21-4.28 (m, 4H) 6.74-6.85 (m, 3H) 7.12-7.16 (m, 1H) 8.20 (d, J=9.09 Hz, 1H) 8.51 (t, J=5.81 Hz, 1H) 8.70 (d, J=5.05 Hz, 1H) 11.28 (s, 1H) ES (M+H)⁺=450.

The intermediates for Example 6 were prepared as follows:

### Intermediate 25: (2S)-1-(tert-Butoxycarbonyl)-5-oxopiperidine-2-carboxylic acid

To a solution of 1-tert-butyl 2-methyl (2S)-5-oxopiperidine-1,2-dicarboxylate (1.28 g) in THF (5 mL), methanol (2 ml) and water (2 mL) was added lithium hydroxide (s) (0.17 g). After heating at bath temperature of 85° C. for 2½ hours, the reaction was diluted with ethyl acetate and 1N HCl. The organic solution was collected, washed with brine, dried over anhydrous sodium sulfate, filtered and concentrated to yield 1.20 g of the title compound.

ES (M−H)⁻=242.

### Intermediate 26: tert-Butyl (2S)-2-{[(6-methoxy-1,5-naphthyridin-4-yl)amino]carbonyl}-5-oxopiperidine-1-carboxylate

To a solution of (2S)-1-(tert-butoxycarbonyl)-5-oxopiperidine-2-carboxylic acid (1.20 g) in THF (15 mL) and DMF (5 mL) were added triethylamine (2 mL), 6-methoxy-1,5-naphthyridin-4-amine (1.01 g) and bromotripyrrolidinophosphonium hexafluorophosphate (PyBroP) (2.6 g). After 3 hours at a bath temperature of 75° C., the reaction was diluted with saturated solution of NaHCO₃ and ethyl acetate. The organic solution was collected and washed with brine, dried over anhydrous sodium sulfate, filtered and concentrated. This residue was purified by flash chromatography using a FlashMaster™ with methylenechloride to dichloromethane/methanol (10:1) as eluants to yield 1.19 g of the title compound.

ES (M+H)⁺=401.

### Intermediate 27: tert-Butyl (2S,5S)-5-[(2,3-dihydro-1,4-benzodioxin-6-ylmethyl)amino]-2-{[(6-methoxy-1,5-naphthyridin-4-yl)amino]carbonyl}piperidine-1-carboxylate

To a solution of tert-butyl (2S)-2-{[(6-methoxy-1,5-naphthyridin-4-yl)amino]carbonyl}-5-oxopiperidine-1-carboxylate (1.19 g) in dichloroethane (50 mL) were added 1-(2,3-dihydro-1,4-benzodioxin-6-yl)methanamine (0.60 g) and sodium cyanoborohydride (NaCNBH₃) (0.42 g). After stirring at room temperature 60 hours, the reaction was concentrated. This residue was purified by flash chromatography using a FlashMaster™ using ethylacetate and hexane as eluants yielding 0.45 g of the title compound.

ES (M+H)⁺=550.

### Example 7

### (2S)-5-[(2,3-Dihydro-1,4-benzodioxin-6-ylmethyl)(methyl)amino]-N-(6-methoxy-1,5-naphthyridin-4-yl)-1-methylpiperidine-2-carboxamide

To a solution of (2S,5S)-5-[(2,3-dihydro-1,4-benzodioxin-6-ylmethyl)amino]-N-(6-methoxy-1,5-naphthyridin-4-yl)piperidine-2-carboxamide (51 mg) (Example 6) in dichloromethane (2 mL) and methanol (0.2 mL) was added paraformaldehyde (0.035 g). After 1 hour at room temperature, NaCNBH₃ (0.035 g) was added. After stirring overnight, additional paraformaldehyde (0.037 g) and NaCNBH₃ (0.039 g) were added. The reaction was concentrated and purified by semi-prep HPLC on a Atlantic C18 5 μM column (A: 0.1% TFA/water; B: 0.1% TFA/acetonitrile; 0-95% B in 15 minute gradient). Fractions containing the product were collected, concentrated slightly, neutralized with Na₂CO₃ (s) and extracted twice with ethyl acetate. The organic solution was washed with brine, dried over anhydrous sodium sulfate, filtered and concentrated to yield 20 mg of the title compound.

¹H NMR (400 MHz, CHLOROFORM-D) δ ppm 1.71-1.80 (m, 1H) 1.85-1.96 (m, 1H) 2.13-2.24 (m, 3H) 2.32-2.42 (m, 1H) 2.54 (s, 3H) 2.55-2.64 (m, 1H) 2.65-2.71 (m, 1H) 3.04-3.14 (m, 21H) 3.38 (s, 1H) 3.56 (s, 1H) 3.96 (s, 3H) 4.13-4.16 (m, 1H) 4.18-4.26 (m, 4H) 6.69-6.81 (m, 3H) 7.13 (d, J=9.09 Hz, 1H) 8.20 (d, J=9.09 Hz, 1H) 8.54 (t, J=5.18 Hz, 1H) 8.68-8.74 (m, 1H) 11.13 (s, 1H).

ES (M+H)⁺=478.

### Example 8

### (2S)-5-[(2,3-Dihydro-1,4-benzodioxin-6-ylmethyl)(methyl)amino]-N-(6-methoxy-1,5-naphthyridin-4-yl)piperidine-2-carboxamide

To a solution of tert-butyl (2S,5S)-5-[(2,3-dihydro-1,4-benzodioxin-6-ylmethyl)amino]-2-{[(6-methoxy-1,5-naphthyridin-4-yl)amino]carbonyl}piperidine-1-carboxylate (0.14 g) (Intermediate 27) in dichloromethane (2 mL) and methanol (0.2 mL) was added paraformaldehyde (0.050 g). After 1 hour at room temperature, NaCNBH₃ (0.040 g) was added. After 1½ hours, TFA (0.40 mL) was added and the reaction warmed to bath temperature of 45° C. After 2 hour, the reaction was concentrated and purified by semi-prepHPLC on an Atlantic C18 5 μM column (A: 0.1% TFA/water; B: 0.1% TFA/acetonitrile; 0-95% B in 15 minute gradient). Fractions containing the product were collected, concentrated slightly, neutralized with Na₂CO₃ (s), and extracted with twice with ethyl acetate. The organic solution was washed with brine, dried over anhydrous sodium sulfate, filtered and concentrated to yield 26 mg of the title compound.

¹H NMR (400 MHz, CHLOROFORM-D) δ ppm 1.59-1.71 (m, 2H) 1.84-1.95 (m, 1H) 2.37-2.47 (m, J=9.54, 6.13, 3.54 Hz, 1H) 2.53-2.64 (m, 1H) 2.87 (dd, J=13.64, 9.60 Hz, 1H) 3.01-3.10 (m, 1H) 3.39-3.50 (m, 21H) 3.64-3.73 (m, 1H) 4.05-4.12 (m, 3H) 4.21 (s, 4H) 6.70-6.81 (m, 3H) 7.15 (d, J=8.84 Hz, 1H) 8.18-8.24 (m, 1H) 8.55 (d, J=5.05 Hz, 1H) 8.68-8.74 (m, 1H) 11.53 (s, 1H).

ES (M+H)⁺=464.

### Example 9

### 5-[(2,3-Dihydro-1,4-benzodioxin-6-ylmethyl)amino]-N-(6-methoxy-1,5-naphthyridin-4-yl)-6-oxopiperidine-2-carboxamide

To a solution of 5-amino-N-(6-methoxy-1,5-naphthyridin-4-yl)-6-oxopiperidine-2-carboxamide (0.5 g) in methanol (10 mL) were added 2,3-dihydro-1,4-benzodioxine-6-carbaldehyde (0.26 g) and 3 A molecular sieves powder (0.12 g) and heated (80° C., 30 minutes.). The solution was chilled with an ice bath and NaCNBH₃ (0.10 g) was added. After stirring at room temperature overnight, the reaction was concentrated. The residue was dissolved in ethyl acetate, washed with saturated solution of NaHCO₃ and brine, dried over anhydrous sodium sulfate, filtered and concentrated. This residue was purified by semi-prepHPLC on an Atlantic C18 5 μM column (A: 0.1% TFA/water; B: 0.1% TFA/acetonitrile; 0-95% B in 15 minute gradient). Fractions containing the product were collected, concentrated slightly, neutralized with Na₂CO₃ (s) and extracted with twice with ethyl acetate. The organic solution was washed with brine, dried over anhydrous sodium sulfate, filtered and concentrated to yield 51 mg of the titled compound as a 1:1 diastereomeric mixture.

¹H NMR (400 MHz, DMSO-D6) δ ppm 1.05-1.30 (m, 1H) 1.45 (m, 1H) 1.62 (m, 1H) 1.86 (m, 1H) 1.94-2.14 (m, 2H) 2.20 (m, 2H) 3.00-3.14 (m, 2H) 3.42-3.77 (m, 4H) 4.02 (s, 3H) 4.08 (s, 3H) 4.14-4.40 (m, 10H) 6.62-6.86 (m, 6H) 7.32 (s, 1H) 7.35 (s, 1H) 8.21 (m, 1H) 8.28 (m, 1H) 8.30 (m, 1H) 8.35 (m, 2H) 8.43 (d, J=5.05 Hz, 1H) 8.72 (m, 2H) 10.18 (s, 1H) 10.30 (s, 1H).

ES (M+H)⁺=464.

The intermediates for Example 9 were prepared as follows:

### Intermediate 28: Ethyl 5-[(tert-butoxycarbonyl)amino]-6-oxopiperidine-2-carboxylate

To a solution of ethyl 5-amino-6-oxopiperidine-2-carboxylate (10.7 g) (J. Org. Chem. (1984), 49(12), 2286-8.) in dioxane (150 mL) and triethylamine (10 mL) were added di-tert-butyldicarbonate (13.6 g) and DMAP (0.37 g). After 4 hours, the reaction was concentrated. The residue was dissolved in ethylacetate, washed with saturated solution of NaHCO₃ and brine, dried over anhydrous sodium sulfate, filtered and concentrated. This residue was purified by flash chromatography using a FlashMaster™ with dichloromethane to dichloromethane/methanol (10:1) as eluants yielding 4.62 g of the title compound.

ES (M+Na)⁺=309.

### Intermediate 29: tert-Butyl 6-(aminocarbonyl)-2-oxopiperidin-3-ylcarbamate

A solution of ethyl 5-[(tert-butoxycarbonyl)amino]-6-oxopiperidine-2-carboxylate (2.24 g) in 7N NH₃/MeOH (10 mL) was heated in microwave (30 minutes, 100° C.). The reaction was concentrated to dryness yielding 2.13 g of the title compound.

ES (M+Na)⁺=258.

### Intermediate 30: tert-Butyl 6-{[(6-methoxy-1,5-naphthyridin-4-yl)amino]carbonyl}-2-oxopiperidin-3-ylcarbamate

To a solution of tert-butyl 6-(aminocarbonyl)-2-oxopiperidin-3-ylcarbamate (1.06 g) in dioxane (15 mL) were added Pd₂(dba)₃ (0.2 g), BINAP (0.12 g), and CsCO₃ (0.85 g). The mixture was degassed by bubbling nitrogen gas into the solution before adding 6-methoxy-1,5-naphthyridin-4-yl trifluoromethanesulfonate (1.40 g). The mixture was heated by microwave (2 hour, 100° C., twice). The mixture was diluted with ethylacetate and water. The organic solution was collected, washed with brine, dried over anhydrous sodium sulfate, filtered and concentrated. This residue was purified by flash chromatography using a FlashMaster™ with methylenechloride to methylenechloride/methanol (10:1) as eluants yielding 1.17 g of the title compound.

ES (M+Na)⁺=416.

### Intermediate 31: 5-Amino-N-(6-methoxy-1,5-naphthyridin-4-yl)-6-oxopiperidine-2-carboxamide

A solution of tert-butyl 6-{[(6-methoxy-1,5-naphthyridin-4-yl)amino]carbonyl}-2-oxopiperidin-3-ylcarbamate (1.17 g) in 30% trifluoroacetic acid (TFA)/dichloromethane (10 mL) was heated at 45 C. After 2 hours, the reaction was concentrated. The mixture was diluted with ethylacetate and saturated solution of NaHCO₃. The organic phase was washed with brine, dried over anhydrous sodium sulfate, filtered and concentrated. Additional material was collected from the aqueous layer by concentrating to dryness. This residue was suspended in hot methanol and filtered. The filtrate was combined with the organic extract and the combined residue was purified by flash chromatography using a FlashMaster™ with dichloromethane to dichloromethane/methanol (10:1) as eluants yielding 1.0 g of the title compound,

ES (M+Na)⁺=316.

### Examples 10-11

### (cis)-5-[(2,3-Dihydro-1,4-benzodioxin-6-ylmethyl)amino]-N-(6-methoxy-1,5-naphthyridin-4-yl)-6-oxopiperidine-2-carboxamide and (trans)-5-[(2,3-dihydro-1,4-benzodioxin-6-ylmethyl)amino]-N-(6-methoxy-1,5-naphthyridin-4-yl)-6-oxopiperidine-2-carboxamide

The diastereomeric isomers of 5-[(2,3-dihydro-1,4-benzodioxin-6-ylmethyl)amino]-N-(6-methoxy-1,5-naphthyridin-4-yl)-6-oxopiperidine-2-carboxamide (Example 9) (45 mg) were separated by semi-prepHPLC (A: 10 mM NH₄OAc, pH 8; B: methanol; 50-95% B in 14 minutes) on a YMC Carotenoid(TM) C30, 5 μM column. Fractions containing the product were collected, concentrated slightly, and extracted with twice with ethyl acetate. The organic solution was washed with brine, dried over anhydrous sodium sulfate, filtered and concentrated to yield 34 mg of the cis and trans products.

Peak 1: 18 mg of (cis)-5-[(2,3-dihydro-1,4-benzodioxin-6-ylmethyl)amino]-N-(6-methoxy-1,5-naphthyridin-4-yl)-6-oxopiperidine-2-carboxamide.

¹H NMR (400 MHz, DMSO-D6) δ ppm 1.50-1.59 (m, 1H) 2.01-2.09 (m, 1H) 2.10-2.19 (m, 1H) 2.23-2.33 (m, 1H) 3.10-3.20 (m, 1H) 3.61 (d, 1H) 3.72 (d, J=13.64 Hz, 1H) 4.08-4.18 (m, 3H) 4.20-4.26 (m, 4H) 4.27-4.35 (m, 1H) 6.72-6.83 (m, 3H) 7.39 (d, J=9.09 Hz, 1H) 8.35 (d, J=8.84 Hz, 1H) 8.41 (s, 1H) 8.48 (d, J=5.05 Hz, 1H) 8.79 (d, J=5.05 Hz, 1H) 10.35 (s, 1H).

ES (M+H)⁺=464.

Peak 2: 16 mg of (trans)-5-[(2,3-dihydro-1,4-benzodioxin-6-ylmethyl)amino]-N-(6-methoxy-1,5-naphthyridin-4-yl)-6-oxopiperidine-2-carboxamide,

¹H NMR (400 MHz, DMSO-D6) δ ppm 1.63 (m, 1H) 1.89 (m, 1H) 2.02 (m, 1H) 2.28 (m, 1H) 3.09 (m, 1H) 3.56-3.67 (d, 1H) 3.67-3.77 (d, 1H) 4.02 (s, 3H) 4.19 (s, 4H) 4.29 (m, 1H) 6.75 (s, 2H) 6.82 (s, 1H) 7.33 (d, J=9.09 Hz, 1H) 8.21 (d, J=1.01 Hz, 1H) 8.29 (d, J=8.84 Hz, 1H) 8.36 (d, J=5.05 Hz, 1H) 8.71 (d, J=5.05 Hz, 1H) 10.18 (s, 1H).

ES (M+H)⁺=464.

### Examples 12-19

The following compounds were synthesized in an analogous method to Example 5:

| | | | ES | |
|---|---|---|---|---|
| Example | Compound | ¹H NMR | (M + H)⁺ | SM |
| 12 | (2S,5R)-5-[(2,3- | (300 MHz, DMSO-D6) δ | 450 | (S)-(+) ethyl-2-pyrrolidone-5- |
| | dihydro[1,4]dioxi- | ppm 1.19-1.54 (m, 2 H) | | carboxylate; 6- |
| | no[2,3-c]pyridin- | 1.94-2.09 (m, 2 H) 2.23-2.36 | | methoxyquinolin-4-yl |
| | 7- | (m, 1 H) 2.35-2.45 | | trifluoromethanesulfonate |
| | ylmethyl)amino]- | (m, 1 H) 3.17 (d, | | (WO 2004035569); and 2,3- |
| | N-(6- | J = 12.81 Hz, 1 H) 3.43 (d, | | dihydro[1,4]dioxino[2,3- |
| | methoxyquinolin- | J = 11.87 Hz, 1 H) 3.69 (s, | | c]pyridine-7-carbaldehyde |
| | 4-yl)piperidine-2- | 2 H) 3.92 (s, 3 H) 4.21- | | |
| | carboxamide | 4.39 (m, 4 H) 6.95 (s, 1 | | |
| | | H) 7.35-7.49 (m, 2 H) | | |
| | | 7.86-7.99 (m, 2 H) 8.00 | | |
| | | (s, 1 H) 8.63 (d, J = 4.90 | | |
| | | Hz, 1 H) | | |
| 13 | (2S,5R)-5-[(2,3- | (300 MHz, | 469 | (S)-(+) ethyl-2-pyrrolidone-5- |
| | dihydro[1,4]dioxi- | CHLOROFORM-D) δ | | carboxylate; 8-bromo-7- |
| | no[2,3-c]pyridin- | ppm 1.38-1.53 (m, 1 H) | | fluoro-2-methoxy-1,5- |
| | 7- | 1.68-1.83 (m, 1 H) 2.03-2.15 | | naphthyridine; and 2,3- |
| | ylmethyl)amino]- | (m, 1H) 2.16-2.28 | | dihydro[1,4]dioxino[2,3- |
| | N-(3-fluoro-6- | (m, J = 13.09, 8.95, | | c]pyridine-7-carbaldehyde |
| | methoxy-1,5- | 3.58 Hz, 1 H) 2.57-2.71 | | |
| | naphthyridin-4- | (m, 2 H) 3.26-3.41 (m, | | |
| | yl)piperidine-2- | J = 8.48 Hz, 1 H) 3.56 (dd, | | |
| | carboxamide | J = 9.51, 3.67 Hz, 1 H) | | |
| | | 3.76-3.90 (m, 2 H) 4.00-4.15 | | |
| | | (m, 3 H) 4.32 (ddd, | | |
| | | J = 15.78, 5.51, 2.07 Hz, 4 | | |
| | | H) 6.82 (s, 1 H) 7.10 (d, | | |
| | | J = 9.04 Hz, 1 H) 8.12 (s, 1 | | |
| | | H) 8.15-8.24 (m, 1H) | | |
| | | 8.70 (d, J = 2.45 Hz, 1 H) | | |
| | | 9.91 (s, 1 H) | | |
| 14 | (2S,5R)-N-(2- | (400 MHz, DMSO-D6) δ | 445 | (S)-(+) ethyl-2-pyrrolidone-5- |
| | cyanoquinolin-8- | ppm 1.08-1.35 (m, 1 H) | | carboxylate; 2-cyanoquinolin- |
| | yl)-5-[(2,3- | 1.36-1.61 (m, 1 H) 1.86-2.14 | | 8-yl trifluoromethanesulfonate |
| | dihydro[1,4]dioxi- | (m, 2 H) 2.30-2.42 | | (WO 2004002992); and 2,3- |
| | no[2,3-c]pyridin- | (m, 1 H) 2.43-2.49 | | dihydro[1,4]dioxino[2,3- |
| | 7- | (m, 1 H) 2.97-3.12 (m, 1 | | c]pyridine-7-carbaldehyde |
| | ylmethyl)amino] | H) 3.19 (d, 1 H) 3.28- | | |
| | piperidine-2- | 3.37 (m, 1 H) 3.70-3.76 | | |
| | carboxamide | (s, 2 H) 4.24-4.32 (m, 2 | | |
| | | H) 4.32-4.38 (m, 2 H) | | |
| | | 7.77-7.84 (m, 2 H) 8.01-8.05 | | |
| | | (m, 1 H) 8.14 (d, | | |
| | | J = 8.34 Hz, 1 H) 8.70 (d, | | |
| | | J = 8.59 Hz, 1 H) 8.77-8.89 | | |
| | | (m, 1 H) 10.72-10.85 | | |
| | | (m, 1 H) | | |
| 15 | (2R,5S)-N-(2- | (400 MHz, | 445 | (R)-(+) methyl-2-pyrrolidone- |
| | cyanoquinolin-8- | CHLOROFORM-D) δ | | 5-carboxylate; 2- |
| | yl)-5-[(2,3- | ppm 1.34-1.56 (m, 1 H) | | cyanoquinolin-8-yl |
| | dihydro[1,4]dioxi- | 1.59-1.76 (m, 1 H) 2.07-2.19 | | trifluoromethanesulfonate; 2,3- |
| | no[2,3-c]pyridin- | (m, 1 H) 2.19-2.32 | | dihydro[1,4]dioxino[2,3- |
| | 7- | (m, 1 H) 2.54-2.66 | | c]pyridine-7-carbaldehyde |
| | ylmethyl)amino] | (m, 1 H) 2.66-2.79 (m, 1 | | |
| | piperidine-2- | H) 3.28-3.57 (m, 2 H) | | |
| | carboxamide | 3.86 (s, 2 H) 4.20-4.45 | | |
| | | (m, 4 H) 6.84 (s, 1 H) | | |
| | | 7.26 (s, 1 H) 7.56 (d, | | |
| | | J = 8.08 Hz, 1 H) 7.63-7.86 | | |
| | | (m, 2 H) 8.13 (s, 1 | | |
| | | H) 8.30 (d, J = 8.34 Hz, 1 | | |
| | | H) 8.92 (d, J = 7.58 Hz, 1 | | |
| | | H) 10.70 (s, 1 H) | | |
| 16 | (2R,5S)-5-[(2,3- | (400 MHz, | 451 | (R)-(+)-methyl-2-pyrrolidone- |
| | dihydro[1,4]dioxi- | CHLOROFORM-D) δ | | 5-carboxylate; 6-methoxy-1,5- |
| | no[2,3-c]pyridin- | ppm 1.37-1.53 (m, 1 H) | | naphthyridin-4-yl |
| | 7- | 1.65-1.78 (m, 1 H) 2.02-2.13 | | trifluoromethanesulfonate; 2,3- |
| | ylmethyl)amino]- | (m, 1 H) 2.18-2.28 | | dihydro[1,4]dioxino[2,3- |
| | N-(6-methoxy- | (m, 1 H) 2.56-2.71 | | c]pyridine-7-carbaldehyde |
| | 1,5-naphthyridin- | (m, 2 H) 3.30 (d, J = 9.35 | | |
| | 4-yl)piperidine-2- | Hz, 1 H) 3.49 (dd, | | |
| | carboxamide | J = 9.47, 3.66 Hz, 1 H) | | |
| | | 3.77-3.89 (m, 2 H) 4.10 | | |
| | | (s, 3 H) 4.24-4.38 (m, 4 | | |
| | | H) 6.82 (s, 1 H) 7.14 (d, | | |
| | | J = 9.09 Hz, 1 H) 8.12 (s, 1 | | |
| | | H) 8.20 (d, J = 9.09 Hz, 1 | | |
| | | H) 8.49 (d, J = 5.05 Hz, 1 | | |
| | | H) 8.69 (d, J = 5.05 Hz, 1 | | |
| | | H) 10.99 (s, 1 H) | | |
| 17 | (2S,5R)-5-[(2,3- | (300 MHz, | 450 | (S)-(+) ethyl-2-pyrrolidone-5- |
| | dihydro-1,4- | CHLOROFORM-D) δ | | carboxylate; 6-methoxy-1,5- |
| | benzodioxin-6- | ppm 1.22-1.47 (m, 2 H) | | naphthyridin-4-yl |
| | ylmethyl)amino]- | 1.60-1.77 (m, 2 H) 1.99-2.11 | | trifluoromethanesulfonate; 2,3- |
| | N-(6-methoxy- | (m, 1 H) 2.14-2.27 | | dihydro-1,4-benzodioxine-6- |
| | 1,5-naphthyridin- | (m, 1 H) 2.51-2.69 | | carbaldehyde |
| | 4-yl)piperidine-2- | (m, 2 H) 3.27 (d, J = 10.93 | | |
| | carboxamide | Hz, 1 H) 3.47 (dd, | | |
| | | J = 9.61, 3.58 Hz, 1 H) | | |
| | | 3.72 (s, 2 H) 4.08 (s, 3 H) | | |
| | | 4.24 (s, 4 H) 6.76-6.86 | | |
| | | (m, 3 H) 7.13 (d, J = 9.04 | | |
| | | Hz, 1 H) 8.18 (d, J = 9.04 | | |
| | | Hz, 1 H) 8.47 (d, J = 4.90 | | |
| | | Hz, 1 H) 8.67 (d, J = 5.09 | | |
| | | Hz, 1 H) 10.96 (s, 1 H) | | |
| 18 | (2S,5R)-N-(6- | (300 MHz, DMSO-D6) 8 | 464 | (S)-(+) ethyl-2-pyrrolidone-5- |
| | methoxy-1,5- | ppm 1.20-1.55 (m, 2 H) | | carboxylate; 6-methoxy-1,5- |
| | naphthyridin-4- | 1.89-2.07 (m, J = 22.51, | | naphthyridin-4-yl |
| | yl)-5-{[(3-oxo- | 13.66 Hz, 2 H) 2.31-2.43 | | trifluoromethanesulfonate; 3- |
| | 3,4-dihydro-2H- | (m, 1 H) 3.04-3.20 | | oxo-3,4-dihydro-2H- |
| | pyrido[3,2- | (m, J = 10.36 Hz, 1 H) | | pyrido[3,2-b][1,4]oxazine-6- |
| | b][1,4]oxazin-6- | 3.72 (s, 2 H) 4.04 (s, 3 H) | | carbaldehyde |
| | yl)methyl]ami- | 4.41 (d, J = 5.65 Hz, 1 H) | | |
| | no}piperidine-2- | 4.60 (s, 3 H) 7.04 (d, | | |
| | carboxamide | J = 8.10 Hz, 1 H) 7.25-7.37 | | |
| | | (m, 2 H) 8.26 (d, | | |
| | | J = 9.04 Hz, 1 H) 8.36 (d, | | |
| | | J = 5.09 Hz, 1 H) 8.67 (d, | | |
| | | J = 4.90 Hz, 1 H) 11.04 (s, | | |
| | | 1 H) 11.19 (s, 1 H) | | |
| 19 | (2S,5R)-5- | (300 MHz, DMSO-D6) δ | 392 | (S)-(+) ethyl-2-pyrrolidone-5- |
| | (benzylamino)-N- | ppm 1.26-1.44 (m, 1 H) | | carboxylate; 6-methoxy-1,5- |
| | (6-methoxy-1,5- | 1.45-1.63 (m, 1 H) 1.92-2.15 | | naphthyridin-4-yl |
| | naphthyridin-4- | (m, 2 H) 2.58 (s, 2 | | trifluoromethanesulfonate; |
| | yl)piperidine-2- | H) 3.12-3.30 (m, J = 9.23 | | benzaldehyde |
| | carboxamide | Hz, 2 H) 3.33-3.54 (m, 2 | | |
| | | H) 3.81 (s, 2H) 4.10 (s, 3 | | |
| | | H) 7.30 (d, J = 6.78 Hz, 1 | | |
| | | H) 7.33-7.49 (m, 5 H) | | |
| | | 8.33 (d, J = 8.85 Hz, 1 H) | | |
| | | 8.44 (d, J = 4.90 Hz, 1 H) | | |
| | | 8.74 (d, J = 4.90 Hz, 1 H) | | |
| | | 11.13 (s, 1 H) | | |

### Example 20

### (4R)-4-[(2,3-dihydro[1,4]dioxino [2,3-c]pyridin-7-ylmethyl)amino]-N-(6-methoxy-1,5-naphthyridin-4-yl)-L-prolinamide

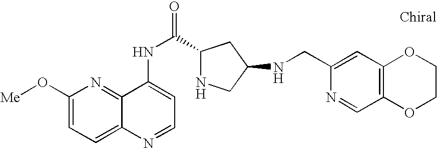

To a solution of N-tert-butoxycarbonyl (4R)-4-[(2,3-dihydro[1,4]dioxino[2,3-c]pyridin-7-ylmethyl)amino]-N-(6-methoxy-1,5-naphthyridin-4-yl)-L-prolinamide in CH₂Cl₂ (3 mL) was added TFA (2 mL). After 15 minutes at room temperature, the reaction was concentrated. The residue was purified by Gilson (A: 0.1% TFA/water; B: 0.1% TFA/acetonitrile). The desired fractions were collected, concentrated slightly, neutralized with Na₂CO₃ (s) and extracted with twice with ethyl acetate. The organic solution was washed with brine, dried (Na₂SO₄), filtered and concentrated to yield 30 mg.

¹H NMR (300 MHz, CHLOROFORM-D) δ ppm 2.10-2.31 (m, 2H) 2.91-3.03 (m, 1H) 3.04-3.14 (m, 1H) 3.39 (s, 1H) 3.75 (s, 2H) 4.11 (s, 3H) 4.15-4.24 (m, 1H) 4.23-4.37 (m, J=13.09, 4.43 Hz, 4H) 6.76 (s, 1H) 7.13 (d, J=8.85 Hz, 1H) 8.10 (s, 1H) 8.18 (d, J=9.04 Hz, 1H) 8.50 (d, J=5.09 Hz, 1H) 8.68 (d, J=5.09 Hz, 1H) 11.74 (s, 1H);

ES (M+H)⁺=437.

The intermediates for Example 20 were prepared as follows:

### Intermediate 32: N-tert-butoxycarbonyl (4R)-4-[(2,3-dihydro[1,4]dioxino[2,3-c]pyridin-7-ylmethyl)amino]-N-(6-methoxy-1,5-naphthyridin-4-yl)-L-prolinamide

To a solution of (4R)-4-[(2,3-dihydro[1,4]dioxino[2,3-c]pyridin-7-ylmethyl)amino]-1-(3,3-dimethylbutanoyl)-L-prolinamide (130 mg) in dioxane (4 mL) were added Pd₂(dba)₃ (20 mg g), BINAP 25 mg g), and CsCO₃ (50 mg). The mixture was degassed by bubbling N₂(g) into the solution before adding 6-methoxy-1,5-naphthyridin-4-yl trifluoromethanesulfonate (120 mg). The mixture was heated by microwave (2 hours, 125° C.). The mixture was diluted with ethylacetate and water. The organic solution was collected, washed with brine, dried (Na₂SO₄), filtered and concentrated. This residue was purified by flash chromatography using a FlashMaster™ yielding 180 mg.

ES (M+Na)⁺=537.

### Intermediate 33: (4R)-4-[(2,3-dihydro[1,4]dioxino[2,3-c]pyridin-7-ylmethyl)amino]-1-(3,3-dimethylbutanoyl)-L-prolinamide

To a solution of (4R)-1-(tert-butoxycarbonyl)-4-[(2,3-dihydro[1,4]dioxino[2,3-c]pyridin-7-ylmethyl)amino]-L-proline (2.4 g) in DMF (50 mL) were added ammonium chloride (2 g), EDC (2.0 g) and HOAT (0.56 g). After stirring overnight at room temperature, the reaction was diluted with ethyl acetate and water. The aqueous layer was collected and extracted six times with ethyl acetate. The combined organic solution was dried (Na₂SO₄), filtered and concentrated. This residue was purified by flash chromatography using a FlashMaster™ yielding 1.1 g.

ES (M+H)⁺=379.

### Intermediate 34: (4R)-1-(tert-butoxycarbonyl)-4-[(2,3-dihydro[1,4]dioxino[2,3-c]pyridin-7-ylmethyl)amino]-L-proline

To a solution of 1-tert-butyl 2-methyl (2S,4R)-4-[(2,3-dihydro[1,4]dioxino[2,3-c]pyridin-7-ylmethyl)amino]pyrrolidine-1,2-dicarboxylate (2.5 g) in THF (45 mL), methanol (5 mL) and water (2 mL) was added 2N lithium hydroxide (5 mL). The mixture was heated for 1½ hours at 40° C. The residue was evaporated repeatedly with methanol to constant weight (2.4 g).

ES (M+H)⁺=380.

### Intermediate 35: To a solution of 1-tert-butyl 2-methyl (2S,4R)-4-[(2,3-dihydro[1,4]dioxino[2,3-c]pyridin-7-ylmethyl)amino]pyrrolidine-1,2-dicarboxylate

A solution of 1-tert-butyl 2-methyl (2S,4R)-4-aminopyrrolidine-1,2-dicarboxylate (3 g) and 2,3-dihydro[1,4]dioxino[2,3-c]pyridine-7-carbaldehyde (2.15 g) in methanol (50 mL) with 3 A molecular sieves powder (0.58 g) was heated at 80° C. for 1 hour. The solution was chilled with an ice bath and NaCNBH₃ (1.0 g) was added. After stirring at room temperature overnight, the reaction was concentrated. The residue was dissolved in ethylacetate, washed with NaHCO₃ (sat'd soln) and brine, dried (Na₂SO₄), filtered and concentrated. The residue was purified by flash chromatography using an ISCO yielding 2.5 g.

ES (M+H)⁺=394.

### Examples 21-23

The following compounds were synthesized in an analogous method to Example 20 involving Intermediate 33 and appropriate Aryl triflate in place of Intermediate 32 Aryl triflate:

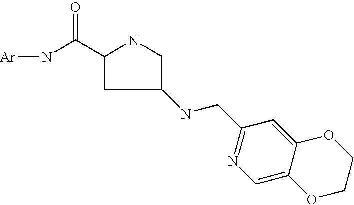

| | | | ES | |
|---|---|---|---|---|
| Example | Compound | ¹H NMR | (M + H)⁺ | Ar SM |
| 21 | (4R)-N-(2- | (300 MHz, | 431 | 2-cyanoquinolin-8-yl |
| | cyanoquinolin-8-yl)- | CHLOROFORM-D) δ | | trifluoromethanesulfonate |
| | 4-[(2,3- | ppm 2.05-2.31 (m, 2 H) | | |
| | dihydro[1,4]dioxino[2,3- | 3.01-3.09 (m, J = 4.33 | | |
| | c]pyridin-7- | Hz, 1 H) 3.09-3.16 (m, 1 | | |
| | ylmethyl)amino]-L- | H) 3.33-3.43 (m, 1 H) | | |
| | prolinamide | 3.77 (s, 2 H) 4.16-4.25 | | |
| | | (m, 1 H) 4.24-4.35 (m, 4 | | |
| | | H) 6.79 (s, 1 H) 7.54 (d, | | |
| | | J = 6.97 Hz, 1 H) 7.63- | | |
| | | 7.77 (m, 2 H) 8.11 (s, 1 | | |
| | | H) 8.28 (d, J = 8.48 Hz, 1 | | |
| | | H) 8.92 (d, J= 9.04 Hz, 1 | | |
| | | H) 11.65 (s, 1 H) | | |
| 22 | (4R)-4-[(2,3- | (400 MHz, | 454 | 8-fluoro-6- |
| | dihydro[1,4]dioxino[2,3- | CHLOROFORM-D) δ | | methoxyquinolin-4-yl |
| | c]pyridin-7- | ppm 2.37-2.47 (m, 1 H) | | trifluoromethanesulfonate |
| | ylmethyl)amino]-N- | 2.49-2.60 (m, 1 H) 3.05 | | (WO2006002047) |
| | (8-fluoro-6- | (dd, J = 13.14, 4.80 Hz, 1 | | |
| | methoxyquinolin-4- | H) 3.46-3.55 (m, 1 H) | | |
| | yl)-L-prolinamide | 3.62-3.71 (m, 1 H) 3.92- | | |
| | | 3.98 (m, 3 H) 4.01- | | |
| | | 4.07 (m, 2 H) 4.25-4.36 | | |
| | | (m, 4H) 4.39-4.49 (m, 1 | | |
| | | H) 6.84 (s, 1 H) 6.93 (s, 1 | | |
| | | H) 7.10-7.17 (m, 1H) | | |
| | | 8.11 (s, 1 H) 8.31 (d, | | |
| | | J = 4.80 Hz, 1 H) 8.70 (d, | | |
| | | J = 5.05 Hz, 1 H) 10.76 | | |
| | | (br. s, 1 H) | | |
| 23 | (4R)-N-(6-cyano-1,7- | (400 MHz, | 432 | Intermediate 36 |
| | naphthyridin-4-yl)-4- | CHLOROFORM-D) δ | | |
| | [(2,3- | ppm 2.27-2.38 (m, 1 H) | | |
| | dihydro[1,4]dioxino[2,3- | 2.50 (m, 1 H) 3.05 (m, 1 | | |
| | c]pyridin-7- | H) 3.56-3.65 (m, 1 H) | | |
| | ylmethyl)amino]-L- | 3.68-3.76 (m, 1 H) 3.97 | | |
| | prolinamide | (m, 1 H) 4.07-4.16 (d, 1 | | |
| | | H) 4.16-4.26 (d, 1 H) | | |
| | | 4.37-4.49 (m, 2 H) 4.55 | | |
| | | (m, 2 H) 7.22 (s, 1 H) | | |
| | | 8.22-8.26 (m, 1 H) 8.51 | | |
| | | (d, J = 5.05 Hz, 1 H) 8.90 | | |
| | | (s, 1 H) 9.06 (d, J =15.31 | | |
| | | Hz, 1 H) 9.46 (s, 1 H) | | |

### Intermediate 36: 6-cyano-1,7-naphthyridin-4-yl trifluoromethanesulfonate

2-Cyano-5-aminopyridine (5.0 g), 2,2-dimethyl-1,3-dioxane-4,6-dione (7.25 g) and triethyl orthoformate (6.9 mL) were refluxed in ethanol (75 mL) for 1.5 hours. This mixture was cooled in an ice bath and filtered and dried to give product (89%).

The product (8.0 g) was then added to refluxing phenyl ether (75 mL) in small portions and stirred for 5 minutes. The mixture was cooled to room temperature and diethyl ether was added and the resulting mixture was filtered. The solids were suspended in ethyl acetate, filtered and dried to give product, 6-Cyano-6-hydroxy-[1,7]-naphthyridine (761 mg).

6-Cyano-6-hydroxy-[1,7]-naphthyridine (700 mg) was added to cooled methylene chloride (70 mL), followed by 2,6-lutidine (0.813 mL), DMAP (70 mg) and trifluoro methanesulfonic anhydride (0.841 mL). The mixture was stirred at 0° C. for 2 hours. The resulting mixture was poured into saturated ammonium chloride solution, washed with brine, dried over sodium sulfate, filtered and concentrated. Purification on silica gel (0-35% Ethyl acetate in hexane) gave the captioned compound (220 mg).

### Example 24

### 6-[({(3S,6S)-6-[2-(3-fluoro-6-methoxy-1,5-naphthyridin-4-yl)ethyl]piperidin-3-yl}amino)methyl]-2H-pyrido[3,2-b][1,4]oxazin-3(4H)-one

To a solution of tert-butyl (2S,5S)-2-[2-(3-fluoro-6-methoxy-1,5-naphthyridin-4-yl)ethyl]-5-{[(3-oxo-3,4-dihydro-2H-pyrido[3,2-b][1,4]oxazin-6-yl)methyl]amino}piperidine-1-carboxylate (0.138 g) in methylene chloride (3 mL) was added trifluoroacetic acid (2 mL). After 2 hours at room temperature, the reaction was concentrated. The residue was purified by flash chromatography using an ISCO yielding 53 mg.

¹H NMR (400 MHz, DMSO-D6) δ ppm 1.24-1.36 (m, 1H) 1.37-1.48 (m, 1H) 1.81-1.92 (m, 1H) 1.93-2.05 (m, 1H) 2.05-2.15 (m, 1H) 2.16-2.27 (m, 1H) 2.54-2.64 (m, 1H) 2.71-2.79 (m, 1H) 2.97-3.06 (m, 1H) 3.15-3.25 (m, 2H) 3.28-3.38 (m, 1H) 3.71-3.82 (m, 2H) 4.07 (s, 3H) 4.63 (s, 2H) 7.04 (d, J=8.34 Hz, 1H) 7.28 (d, J=9.09 Hz, 1H) 7.35 (d, J=8.08 Hz, 1H) 8.31 (d, J=9.09 Hz, 1H) 8.82 (s, 1H) 11.19 (s, 1H).

ES (M+H)⁺=467.

### Intermediate 37: tert-butyl (2S,5S)-2-[2-(3-fluoro-6-methoxy-1,5-naphthyridin-4-yl)ethyl]-5-{[(3-oxo-3,4-dihydro-2H-pyrido[3,2-b][1,4]oxazin-6-yl)methyl]amino}piperidine-1-carboxylate

To a solution of tert-butyl (2S,5S)-5-amino-2-[2-(3-fluoro-6-methoxy-1,5-naphthyridin-4-yl)ethyl]piperidine-1-carboxylate (0.13 g) in methanol (5 mL) were added 3-oxo-3,4-dihydro-2H-pyrido[3,2-b][1,4]oxazine-6-carbaldehyde (68 mg) and 3 A molecular sieves powder (75 mg) was heated at 80° C. for 1 hour. The solution was chilled with an ice bath and NaCNBH₃ (40 mg) was added. After stirring at rt. overnight, the reaction was concentrated. The residue was dissolved in ethylacetate, washed with NaHCO_{3 (sat'd soln)} and brine, dried (Na₂SO₄), filtered and concentrated. The residue was purified by flash chromatography using an ISCO yielding 0.14 g,

ES (M+H)⁺=567.

### Intermediate 38: tert-butyl (2S,5S)-5-amino-2-[2-(3-fluoro-6-methoxy-1,5-naphthyridin-4-yl)ethyl]piperidine-1-carboxylate

To a solution of tert-butyl (2S,5S)-5-azido-2-[2-(3-fluoro-6-methoxy-1,5-naphthyridin-4-yl)ethyl]piperidine-1-carboxylate (0.31 g) in methanol (4 mL) were added 10% Pd/C (70 mg), ZnBr₂ (10 mg) and H₂ (g) balloon. After stirring overnight, the reaction was degassed and filtered through celite to yield 0.26 g.

ES (M+H)⁺=405.

### Intermediate 39: tert-butyl (2S,5S)-5-azido-2-[2-(3-fluoro-6-methoxy-1,5-naphthyridin-4-yl)ethyl]piperidine-1-carboxylate

To a solution of tert-butyl (2S,5R)-2-[2-(3-fluoro-6-methoxy-1,5-naphthyridin-4-yl)ethyl]-5-hydroxypiperidine-1-carboxylate (0.80 g) in methylene chloride (8 mL) at 0° C. were added DIEA (0.70 mL) and methanesulfonyl chloride (0.30 mL). After 2 hours, the reaction was diluted with methylene chloride, washed with NaHCO_{3 (sat'd soln)} and brine, dried (Na₂SO₄), filtered and concentrated. The residue was dissolved in DMF (10 mL) and sodium azide (1.35 g) heated by microwave for 1 hour at 100° C. The mixture was diluted with water and ethylacetate. The organic solution was collected, washed with a saturated solution of NaHCO₃ and brine, dried (Na₂SO₄), filtered, and concentrated. The residue was purified by flash chromatography using an ISCO yielding 0.31 g of tert-butyl (2S,5S)-5-azido-2-[2-(3-fluoro-6-methoxy-1,5-naphthyridin-4-yl)ethyl]piperidine-1-carboxylate (ES (M+H)⁺=431), 85 mg of tert-butyl (2S,5R)-5-azido-2-[2-(3-fluoro-6-methoxy-1,5-naphthyridin-4-yl)ethyl]piperidine-1-carboxylate (ES (M+H)⁺=431), and 15 mg of tert-butyl (2R)-2-[2-(3-fluoro-6-methoxy-1,5-naphthyridin-4-yl)ethyl]-3,4-dihydropyridine-1(2H)-carboxylate.

ES (M+H)⁺=388.

### Intermediate 40: tert-butyl (2S,5R)-2-[2-(3-fluoro-6-methoxy-1,5-naphthyridin-4-yl)ethyl]-5-hydroxypiperidine-1-carboxylate

To a solution of tert-butyl (2S,5R)-5-{[tert-butyl(dimethyl)silyl]oxy}-2-[2-(3-fluoro-6-methoxy-1,5-naphthyridin-4-yl)ethyl]piperidine-1-carboxylate (2.5 g) in THF (30 mL) was added 1 M terabutylammonium fluoride (TBAF) (8 mL). After stirring overnight, the reaction was concentrated. The residue was dissolved in ethylacetate, washed with a saturated solution of NaHCO₃ and brine, dried (Na₂SO₄), filtered and concentrated. The residue was purified by flash chromatography using an ISCO chromatography system yielding 1.01 g.

ES (M+H)⁺=406.

### Intermediate 41: tert-butyl (2S,5R)-5-{[tert-butyl(dimethyl)silyl]oxy}-2-[2-(3-fluoro-6-methoxy-1,5-naphthyridin-4-yl)ethyl]piperidine-1-carboxylate

To a solution of tert-butyl (2R,5R)-5-{[tert-butyl(dimethyl)silyl]oxy}-2-vinylpiperidine-1-carboxylate (2.09 g) in THF (20 mL) at 0° C. was added 0.5 M 9-BBN (25 mL). The ice bath was removed and the resulting reaction mixture was stirred at room temperature for 1 hour. The reaction mixture was covered with foil and 3 M K₃PO₄ (2.2 mL) was added followed by 8-bromo-7-fluoro-2-methoxy-1,5-naphthyridine (1.7 g) in DMF (25 mL). The solution was degassed with N₂ and PdCl₂(dppf)₂ (0.28 g) was added. After stirring overnight, the solution was diluted with ethylacetate, washed with a saturated solution of NaHCO₃ and brine, dried (Na₂SO₄), filtered and concentrated yielding 2.5 g.

ES (M+H)⁺=520.

### Intermediate 42: tert-butyl (2R,5R)-5-{[tert-butyl(dimethyl)silyl]oxy}-2-vinylpiperidine-1-carboxylate

To a suspension of Zn dust (12.46 g) in THF (100 mL) was added diiodomethane (8 mL). The mixture was cooled with an ice bath and trimethylaluminium (6.5 mL) was added. The ice bath was removed and the reaction over time (approximately 15 minutes) became exothermic. The reaction was stirred for an additional 15 additional before it was cooled with an ice bath and tert-butyl (2R,5R)-5-{[tert-butyl(dimethyl)silyl]oxy}-2-formylpiperidine-1-carboxylate (7.1 g) in THF (60 mL) was added. After 6 hours, the slurry was filtered through a celite bed. The filtrate was quenched with a saturated solution of NaHCO₃ and ethylacetate. The organic solution was collected, washed with NaHCO_{3 (sat'd soln)} and brine, dried (Na₂SO₄), filtered and concentrated. The residue was purified by flash chromatography using an ISCO chromatography system yielding 2.23 g.

ES (M+H)⁺=342.

### Intermediate 43: tert-butyl (2R,5R)-5-{[tert-butyl(dimethyl)silyl]oxy}-2-formylpiperidine-1-carboxylate

To a solution of tert-butyl (2R,5R)-5-{[tert-butyl(dimethyl)silyl]oxy}-2-(hydroxymethyl)piperidine-1-carboxylate (7.2 g) in methylene chloride (100 mL) was added Dess-Martin periodinane (10.2 g). After 2 hours at room temperature, the reaction was diluted with methylene chloride, washed with a saturated solution of NaHCO₃ and brine, dried (Na₂SO₄), filtered and concentrated yielding (7.1 g).

ES (M+H)⁺=344

### Intermediate 44: tert-butyl (2R,5R)-5-{[tert-butyl(dimethyl)silyl]oxy}-2-(hydroxymethyl)piperidine-1-carboxylate

To a solution of 1-tert-butyl 2-ethyl (2R,5R)-5-{[tert-butyl(dimethyl)silyl]oxy}piperidine-1,2-dicarboxylate (9.4 g) in THF (100 mL) in an ice bath was added 1 M LAH (24 mL) via an addition funnel. After stirring for 1 hour after addition, the reaction was diluted with ethylacetate and quenched with 1 N HCl. The organic solution was collected, washed with a saturated solution of NaHCO₃ and brine, dried (Na₂SO₄), filtered and concentrated yielding (7.2 g).

ES (M+H)⁺=346.

### Intermediate 45: 1-tert-butyl 2-ethyl (2R,5R)-5-{[tert-butyl(dimethyl)silyl]oxy}piperidine-1,2-dicarboxylate

To a solution of 1-tert-butyl 2-ethyl (2R,5R)-5-hydroxypiperidine-1,2-dicarboxylate (12.4 g) (prepared similar to Intermediate 17 from (S)-(+) ethyl-2-pyrrolidone-5-carboxylate) in DMF (95 mL) was added imidazole (3.1 g) and tBDMSCl (8.2 g). After stirring overnight, additional tBDMSCl (2 g) was added. After 12 hours, the reaction was diluted with methylene chloride, washed with 1 M HCl, a saturated solution of NaHCO₃ and brine, dried (Na₂SO₄), filtered and concentrated. The residue was purified by flash chromatography using an ISCO yielding 9.4 g.

ES (M+H)⁺ 388

### Examples 25-37

The following compounds were synthesized in an analogous method to Example 24 involving Intermediate 42 or it's enantiomer (prepared in a similar manner as Intermediate 45 starting with (R)-(+)-methyl-2-pyrrolidone-5-carboxylate). The examples involve reaction with appropriate Aryl triflate or Aryl bromide in making Intermediate 41 and aldehyde in making Intermediate 37:

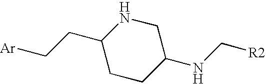

| | | | ES | |
|---|---|---|---|---|
| Example | Compound | ¹H NMR | (M + H)⁺ | SM |
| 25 | (3S,6S)-N-(2,3- | (300 MHz, DMSO-D6) δ | 454 | Aryl: 8-bromo-7-fluoro-2- |
| | dihydro[1,4]dioxino[2,3- | ppm 1.49-1.74 (m, 2 H) | | methoxy-1,5-naphthyridine |
| | c]pyridin-7- | 1.84-1.98 (m, J = 14.88 | | Aldehyde: 2,3- |
| | ylmethyl)-6-[2-(3- | Hz, 2 H) 2.00-2.17 (m, | | dihydro[1,4]dioxino[2,3- |
| | fluoro-6-methoxy- | J = 13.38 Hz, 2 H) 2.21- | | c]pyridine-7-carbaldehyde |
| | 1,5-naphthyridin- | 2.38 (m, 1 H) 3.11-3.26 | | |
| | 4- | (m, 1 H) 4.08 (s,3 H) | | |
| | yl)ethyl]piperidin- | 4.24 (s, 2 H) 4.29-4.47 | | |
| | 3-amine | (m, 4 H) 7.18 (s, 1 H) | | |
| | | 7.22-7.30 (m, 1 H) 8.18 | | |
| | | (s, 1 H) 8.30 (d, J = 8.85 | | |
| | | Hz, 1 H) 8.81 (s, 1 H) | | |
| | | 9.46-9.81 (m, 2 H) | | |
| 26 | (3S,6S)-6-[2-(3- | (300 MHz, DMSO-D6) δ | 473 | Aryl: 8-bromo-7-chloro-2- |
| | chloro-6-methoxy- | ppm 1.57-1.76 (m, | | methoxy-1,5-naphthyridine |
| | 1,5-naphthyridin- | J = 9.48, 9.48, 9.48 Hz, 2 | | Aldehyde: (2E)-3-(2,5- |
| | 4-yl)ethyl]-N- | H) 1.80-2.15 (m, 2 H) | | difluorophenyl)acrylaldehyde |
| | [(2E)-3-(2,5- | 2.22-2.38 (m, 2 H) 2.95- | | (FR2872164) |
| | difluorophenyl)prop- | 3.25 (m, 2 H) 3.25- | | |
| | 2-en-1- | 3.39 (m, 2 H) 3.47-3.58 | | |
| | yl]piperidin-3- | (m, 1 H) 3.61-3.73 (m, | | |
| | amine | J = 11.11 Hz, 1 H) 3.88 | | |
| | | (d, J = 4.52 Hz, 2 H) 4.08 | | |
| | | (s, 3 H) 6.42-6.59 (m, 1 | | |
| | | H) 6.96 (d, J = 16.20 Hz, | | |
| | | 1 H) 7.16-7.36 (m, 2 H) | | |
| | | 7.43-7.53 (m, 1 H) 8.30 | | |
| | | (d, J = 9.04 Hz, 1 H) 8.78 | | |
| | | (s, 1 H) 9.54-9.91 (m, 2 H) | | |
| 27 | 6-[({(3S,6S)-6-[2- | (400 MHz, DMSO-D6) δ | 483 | Aryl: 8-bromo-7-chloro-2- |
| | (3-chloro-6- | ppm 1.68 (m, 2 H) 1.91 | | methoxy-1,5-naphthyridine |
| | methoxy-1,5- | (m, 1 H) 2.02-2.12 (m, | | Aldehyde: 3-oxo-3,4-dihydro- |
| | naphthyridin-4- | 2 H) 2.23-2.40 (m, 2 H) | | 2H-pyrido[3,2-b][1,4]oxazine-6- |
| | yl)ethyl]piperidin- | 3.05 (m, 1 H) 3.17 (m, 1 | | carbaldehyde |
| | 3- | H) 3.35 (t, J =8.08 Hz, 2 | | |
| | yl}amino)methyl]- | H) 3.62-3.74 (m, 2 H) | | |
| | 2H-pyrido[3,2- | 4.10 (s, 3 H) 4.25 (s, 2 | | |
| | b][1,4]oxazin- | H) 4.71 (s, 2 H) 7.23 (d, | | |
| | 3(4H)-one | J = 8.08 Hz, 1 H) 7.30 (d, | | |
| | | 1 H) 7.46 (d, J = 8.08 Hz, | | |
| | | 1 H) 8.29-8.34 (d, 1 H) | | |
| | | 8.80 (s, 1 H) 11.37 (s, 1 H) | | |
| 28 | (3S,6S)-6-[2-(3- | (300 MHz, DMSO-D6) δ | 449 | Aryl: 8-bromo-7-chloro-2- |
| | chloro-6-methoxy- | ppm 1.53-1.73 (m, 2 H) | | methoxy-1,5-naphthyridine |
| | 1,5-naphthyridin- | 1.79-2.09 (m, 2 H) 2.23- | | Aldehyde: 2,3- |
| | 4-yl)ethyl]-N-(2,3- | 2.37 (m, 2 H) 2.95- | | dihydro[1,4]dioxino[2,3- |
| | dihydro[1,4]dioxino[2,3- | 3.21 (m, 2 H) 3.31 (t, | | c]pyridine-7-carbaldehyde |
| | c]pyridin-7- | J = 7.72 Hz, 2 H) 3.43- | | |
| | ylmethyl)piperidin- | 3.55 (m, 1 H) 3.57-3.69 | | |
| | 3-amine | (m, J= 13.28, 4.62 Hz, 1 | | |
| | | H) 4.07 (s, 3 H) 4.25 (s, | | |
| | | 2 H) 4.29-4.42 (m, 4 H) | | |
| | | 7.17 (s, 1 H) 7.31 (d, | | |
| | | J = 9.04 Hz, 1 H) 8.18 (s, | | |
| | | 1 H) 8.30 (d, J = 9.04 Hz, | | |
| | | 1 H) 8.78 (s, 1 H) 9.45- | | |
| | | 9.75 (m, 2 H) | | |
| 29 | 6-[({(3S,6S)-6-[2- | (400 MHz, DMSO-D6) δ | 499 | Aryl: 8-bromo-7-chloro-2- |
| | (3-chloro-6- | ppm 1.78 (m, 2 H) 1.95 | | methoxy-1,5-naphthyridine |
| | methoxy-1,5- | (m, 1 H) 2.14 (m, 1 H) | | Aldehyde: 3-oxo-3,4-dihydro- |
| | naphthyridin-4- | 2.41 (m, 2 H) 3.13 (m, 1 | | 2H-pyrido[3,2-b][1,4]thiazine-6- |
| | yl)ethyl]piperidin- | H) 3.28 (m, 1 H) 3.41 | | carbaldehyde |
| | 3- | (m, 2 H) 3.67 (s, 2 H) | | |
| | yl}amino)methyl]- | 4.16 (s, 3 H) 4.37 (m, 2 | | |
| | 2H-pyrido[3,2- | H) 7.30 (d, J = 8.08 Hz, 1 | | |
| | b][1,4]thiazin- | H) 7.39 (d, J = 8.84 Hz, 1 | | |
| | 3(4H)-one | H) 7.98 (d, J = 7.83 Hz, 1 | | |
| | | H) 8.38 (d, J = 9.09 Hz, 1 | | |
| | | H) 8.86 (s, 1 H) 9.57 (s, | | |
| | | 1 H) 9.77 (s, 1 H) 11.08 | | |
| | | (s, 1 H) | | |
| 30 | 8-[2-((2S,5S)-5- | (300 MHz, | 443 | Aryl: 2-cyanoquinolin-8-yl |
| | {[(3-oxo-3,4- | CHLOROFORM-D) δ | | trifluoromethanesulfonate |
| | dihydro-2H- | ppm 1.16 - 1.32 (m, 4 H) | | Aldehyde: 3-oxo-3,4-dihydro- |
| | pyrido[3,2- | 1.76-1.90 (m, 3 H) 2.04 | | 2H-pyrido[3,2-b][1,4]oxazine-6- |
| | b][1,4]oxazin-6- | (m, 2 H) 2.30-2.40 (m, | | carbaldehyde |
| | yl)methyl]amino} | 3H) 2.66 (m, 1 H) 3.15- | | |
| | piperidin-2- | 3.30 (m, 1 H) 3.33-3.49 | | |
| | yl)ethyl]quinoline- | (m, 2 H) 3.82 (s, 2 H) | | |
| | 2-carbonitrile | 4.63 (s, 2 H) 6.91 (d, | | |
| | | J = 8.10 Hz, 1 H) 7.19 (d, | | |
| | | J = 8.10 Hz, 1 H) 7.62- | | |
| | | 7.76 (m, 4 H) 8.28 (d, | | |
| | | J = 8.29 Hz, 1 H) | | |
| 31 | 8-(2-{(2S,5S)-5- | (300 MHZ, DMSO-D6) δ | 430 | Aryl: 2-cyanoquinolin-8-yl |
| | [(2,3- | ppm 1.46-1.76 (m, 2 H) | | trifluoromethanesulfonate |
| | dihydro[1,4]dioxino[2,3- | 1.86-2.17 (m, 2 H) 2.20- | | Aldehyde: 2,3- |
| | c]pyridin-7- | 2.35 (m, 2 H) 2.96- | | dihydro[1,4]dioxino[2,3- |
| | ylmethyl)amino]piperidin-2- | 3.12 (m, 2 H) 3.30 (t, | | c]pyridine-7-carbaldehyde |
| | yl}ethyl)quinoline- | J = 7.82 Hz, 2 H) 3.42- | | |
| | 2-carbonitrile | 3.56 (m, 1 H) 3.57-3.70 | | |
| | | (m, 1 H) 4.26 (s, 2 H) | | |
| | | 4.30-4.42 (m, 4 H) 7.42 | | |
| | | (s, 1 H) 7.72-7.81 (m, 2 | | |
| | | H) 7.80-7.88 (m, 2H) | | |
| | | 8.50 (s, 1 H) 9.44-9.85 | | |
| | | (m, 2 H) | | |
| 32 | (3R,6R)-6-[2-(3- | (300 MHz, DMSO-D6) δ | 473 | Aryl: 8-bromo-7-chloro-2- |
| | chloro-6-methoxy- | ppm 1.56-1.78 (m, 2 H) | | methoxy-1,5-naphthyridine |
| | 1,5-naphthyridin- | 1.80-2.17 (m, 2 H) 2.26- | | Aldehyde: (2E)-3-(2,5- |
| | 4-yl)ethyl]-N- | 2.39 (m, J = 10.74 Hz, 2 | | difluorophenyl)acrylaldehyde |
| | [(2E)-3-(2,5- | H) 2.92-3.11 (m, 1 H) | | (FR2872164) |
| | difluorophenyl)prop- | 3.27-3.35 (m, 2 H) 3.46- | | |
| | 2-en-1- | 3.72 (m, 2 H) 3.87 (d, | | |
| | yl]piperidin-3- | J = 5.46 Hz, 2 H) 4.08 (s, | | |
| | amine | 3 H) 6.39-6.61 (m, 1H) | | |
| | | 6.96 (d, J = 16.01 Hz, 1 | | |
| | | H) 7.18-7.36 (m, 3 H) | | |
| | | 7.42-7.54 (m, 1 H) 8.29 | | |
| | | (d, J = 9.04 Hz, 1 H) 8.78 | | |
| | | (s, 1H) 9.64-9.99 (m, 2 H) | | |
| 33 | (3R,6R)-6-[2-(3- | (300 MHz, DMSO-D6) δ | 470 | Aryl: 8-bromo-7-chloro-2- |
| | chloro-6-methoxy- | ppm 1.50-1.76 (m, 2 H) | | methoxy-1,5-naphthyridine |
| | 1,5-naphthyridin- | 1.78-2.12 (m, 2 H) 2.24- | | Aldehyde: 2,3- |
| | 4-yl)ethyl]-N-(2,3- | 2.38 (m, 2 H) 3.25- | | dihydro[1,4]dioxino[2,3- |
| | dihydro[1,4]dioxino[2,3- | 3.38 (m, J = 9.42, 6.41 | | c]pyridine-7-carbaldehyde |
| | c]pyridin-7- | Hz, 2 H) 3.41-3.56 (m, | | |
| | ylmethyl)piperidin- | J = 12.62, 12.62 Hz, 1 H) | | |
| | 3-amine | 3.57-3.71 (m, 1 H) 4.08 | | |
| | | (s, 3 H) 4.26 (s, 2 H) | | |
| | | 4.30-4.42 (m, 4 H) 7.10- | | |
| | | 7.22 (m, J = 2.45 Hz, 1 | | |
| | | H) 7.31 (d, J = 9.23 Hz, 1 | | |
| | | H) 8.21 (s, 1 H) 8.30 (d, | | |
| | | J = 9.04 Hz, 1 H) 8.79 (s, | | |
| | | 1 H) 9.35-9.76 (m, 2 H) | | |
| 34 | 6-[({(3R,6R)-6-[2- | (300 MHz, DMSO-D6) δ | 499 | Aryl: 8-bromo-7-chloro-2- |
| | (3-chloro-6- | ppm 1.73 (m, 2 H) 1.88 | | methoxy-1,5-naphthyridine |
| | methoxy-1,5- | (m, 1 H) 2.00-2.15 (m, | | Aldehyde: 3-oxo-3,4-dihydro- |
| | naphthyridin-4- | 1 H) 2.35 (m, 2 H) 3.15 | | 2H-pyrido[3,2-b][1,4]thiazine-6- |
| | yl)ethyl]piperidin- | (m, 1 H) 3.28-3.43 (m, | | carbaldehyde |
| | 3- | 3 H) 3.76 (s, 2 H) 4.09 | | |
| | yl}amino)methyl]- | (s, 3 H) 4.27 (m, 2 H) | | |
| | 2H-pyrido[3,2- | 7.26 (d, J = 7.91 Hz, 1 H) | | |
| | b][1,4]thiazin- | 7.31 (d, J = 9.04 Hz, 1 H) | | |
| | 3(4H)-one | 7.90 (d, J = 7.72 Hz, 1 H) | | |
| | | 8.30 (d, J = 9.04 Hz, 1 H) | | |
| | | 8.78 (s, 1 H) 9.72 (s, 1 | | |
| | | H) 9.88 (s, 1 H) 11.04 (s, | | |
| | | 1 H) | | |
| 35 | 6-[({(3R,6R)-6-[2- | (400 MHz, DMSO-D6) δ | 483 | Aryl: 8-bromo-7-chloro-2- |
| | (3-chloro-6- | ppm 1.74 (m, 2 H) 1.91 | | methoxy-1,5-naphthyridine |
| | methoxy-1,5- | (m, 1 H) 2.08 (m, 1 H) | | Aldehyde: 3-oxo-3,4-dihydro- |
| | naphthyridin-4- | 2.31-2.42 (m, 2 H) 3.10 | | 2H-pyrido[3,2-b][1,4]oxazine-6- |
| | yl)ethyl]piperidin- | (m, 1 H) 3.17 (m, 1 H) | | carbaldehyde |
| | 3- | 3.31-3.42 (m, 2 H) 3.71 | | |
| | yl}amino)methyl]- | (m, 2 H) 4.10 (s, 3 H) | | |
| | 2H-pyrido[3,2- | 4.24 (m, 2 H) 4.71 (s, 2 | | |
| | b][1,4]oxazin- | H) 7.25 (d, J = 8.08 Hz, 1 | | |
| | 3(4H)-one | H) 7.30-7.36 (dm, 1 H) | | |
| | | 7.46 (d, J = 8.08 Hz, 1 H) | | |
| | | 8.32 (d, J = 9.09 Hz, 1 H) | | |
| | | 8.80 (s, 1 H) 9.65 (m, 2 | | |
| | | H) 9.74 (m, 1 H) 11.36 | | |
| | | (s, 1 H) | | |
| 36 | 6-[({(3S,6S)-6-[2- | (300 MHz, DMSO-D6) δ | 470 | Aryl: 4-bromo-3-chloro-6- |
| | (3-chloro-6- | ppm 1.58-1.85 (m, 2 H) | | fluoroquinoline (WO2004058144) |
| | fluoroquinolin-4- | 1.89-2.04 (m, 1 H) 2.17- | | Aldehyde: 3-oxo-3,4-dihydro- |
| | yl)ethyl]piperidin- | 2.31 (m, J = 13.56 Hz, 1 | | 2H-pyrido[3,2-b][1,4]oxazine-6- |
| | 3- | H) 2.30-2.41 (m, | | carbaldehyde |
| | yl}amino)methyl]- | J = 10.93 Hz, 1 H) 3.04 - | | |
| | 2H-pyrido[3,2- | 3.21 (m, 1 H) 3.23-3.43 | | |
| | b][1,4]oxazin- | (m, 3 H) 3.61-3.78 (m, | | |
| | 3(4H)-one | 2 H) 4.22 (s, 2 H) 4.69 | | |
| | | (s, 2 H) 7.27 (d, J = 8.10 | | |
| | | Hz, 1 H) 7.44 (d, J = 7.91 | | |
| | | Hz, 1 H) 7.66-7.81 (m, | | |
| | | 1 H) 8.00-8.21 (m, 2 H) | | |
| | | 8.86 (s, 1 H) 9.79-10.06 | | |
| | | (m, 3 H) 11.38 (s, 1 H) | | |
| 37 | 7-chloro-8-[2- | (300 MHz, DMSO-D6) δ | 477 | Aryl: Intermediate 46 |
| | ((2S,5S)-5-{[(3- | ppm 1.17-1.42 (m, 2 H) | | Aldehyde: 3-oxo-3,4-dihydro- |
| | oxo-3,4-dihydro- | 1.54-1.76 (m, 2 H) 1.78- | | 2H-pyrido[3,2-b][1,4]oxazine- |
| | 2H-pyrido[3,2- | 1.96 (m, J = 11.68 Hz, 2 | | 6-carbaldehyde |
| | b][1,4]oxazin-6- | H) 1.95-2.15 (m, 2 H) | | |
| | yl)methyl]amino} | 2.93-3.07 (m, 2 H) 4.23 | | |
| | piperidin-2- | (s, 2 H) 4.69 (s, 2 H) | | |
| | yl)ethyl]-1,5- | 7.22 (d, J = 7.16 Hz, 1 H) | | |
| | naphthyridine-2- | 7.45 (d, J = 6.97 Hz, 1 H) | | |
| | carbonitrile | 8.38 (d, J = 9.04 Hz, 1 H) | | |
| | | 8.76 (d, J = 9.61 Hz, 1 H) | | |
| | | 9.19 (s, 1 H) 9.34-9.87 | | |
| | | (m, 3 H) 11.36 (s, 1 H) | | |
| 38 | 7-fluoro-8-[2- | (300 MHz, DMSO-D6) δ | 462 | Aryl: Intermediate 47 |
| | ((2R,5R)-5-{[(3- | ppm 1.11-1.59 (m, 3 H) | | Aldehyde: 3-oxo-3,4-dihydro- |
| | oxo-3,4-dihydro- | 1.77-1.95 (m, 2 H) 1.96- | | 2H-pyrido[3,2-b][1,4]oxazine- |
| | 2H-pyrido[3,2- | 2.29 (m, 3 H) 2.81- | | 6-carbaldehyde |
| | b][1,4]oxazin-6- | 3.07 (m, .J = 13.56 Hz, 2 | | |
| | yl)methyl]amino} | H) 3.09-3.24 (m, | | |
| | piperidin-2- | J = 14.88 Hz, 2 H) 3.81 (s, | | |
| | yl)ethyl]-1,5- | 2 H) 4.62 (s, 2 H) 7.06 | | |
| | naphthyridine-2- | (d, J = 3.96 Hz, 1 H) 7.34 | | |
| | carbonitrile | (d, J = 5.65 Hz, 1 H) 8.34 | | |
| | | (d, J = 8.29 Hz, 1 H) 8.76 | | |
| | | (d, J = 7.72 Hz, 1 H) 9.24 | | |
| | | (s, 1 H) 11.23 (s, 1 H) | | |

### Intermediate 46: 8-bromo-7-chloro-1,5-naphthyridine-2-carbonitrile

To a solution of 5-amino-2-methoxypyridine (11 g) in ethanol (150 mL) was added 2,2-dimethyl-1,3-dioxane-4,6-dione (16 g) and triethyl orthoformate (15 mL). After heating at reflux for 2 hours, the mixture was cooled in an ice bath, filtered and the solids were washed with methanol to give 5-{[(6-methoxypyridin-3-yl)amino]methylene}-2,2-dimethyl-1,3-dioxane-4,6-dione (23 g).

5-{[(6-Methoxypyridin-3-yl)amino]methylene}-2,2-dimethyl-1,3-dioxane-4,6-dione (23 g) was added in small portions to refluxing phenyl ether and stirred for 25 minutes. The mixture was then cooled to room temperature and diethyl ether (1000 mL) was added. The solids were filtered, suspended in diethyl ether, stirred at room temperature for 30 minutes and filtered again to give 4-hydroxy-6-methoxy-1,5-naphthyridine (10.4 g).

4-Hydroxy-6-methoxy-1,5-naphthyridine (10.4 g) and N-chlorosuccinimide (8.7 g) were combined and heated at 65° C. in acetic acid (120 mL) for 1.5 hours. The mixture was cooled to room temperature, filtered, washed with water, saturated sodium bicarbonate solution, then with water again, and dried in vacuo to give product 3-chloro-4-hydroxy-6-methoxy-1,5-naphthyridine (12 g).

3-Chloro-4-hydroxy-6-methoxy-1,5-naphthyridine (12 g) was heated at reflux in phosphorous oxychloride (1000 mL) for 1 hour. The mixture was cooled in an ice bath and water (140 mL) was added slowly. The resulting mixture was refluxed for 10 minutes, then cooled to from temperature. Sodium hydroxide was used to adjust the pH to 7 whereby solids crashed out of solution. The mixture was filtered and dried in vacuo to give 3-chloro-4-chloro-6-hydroxy-1,5-naphthyridine (10 g).

To a solution of 3-chloro-4-chloro-6-hydroxy-1,5-naphthyridine (10 g) in methylene chloride (100 mL) at 0° C. was added 2,6-lutidine (9.0 mL), DMAP (1.0 g) and trifluoro methanesulfonic anhydride (10 mL). After 4 hours, the mixture was poured into saturated ammonium chloride solution, layers separated and the organics were washed with brine, dried over sodium sulfate, filtered and concentrated. Purification on silica gel column (0-5% methanol in methylene chloride eluent) gave yellow solid as product triflate (11.3 g).

To a solution of the triflate (1.4 g) in DMF (15 mL) was added zinc cyanide (1.2 g) and tetrakis (triphenylphosphine) palladium (500 mg). The mixture was heated in a microwave reactor at 115° C. for 15 minutes after which the mixture was diluted with ethyl acetate and water, washed with brine, dried over sodium sulfate and concentrated to give crude 7,8-dichloro-1,5-naphthyridine-2-carbonitrile (10 g).

To the product above (9.0 g) was heated at reflux in acetic acid (100 mL) overnight. After cooling to room temperature, the mixture was filtered, washed with diethyl ether and dried under reduced pressure to give 7-chloro-8-hydroxy-1,5-naphthyridine-2-carbonitrile (4.2 g) To a solution of the product above (300 mg) in DMF (10 mL) at 0° C. was added phosphorous tribromide (0.219 mL). The reaction was stirred for 30 minutes at 0° C. and then 1.5 hours at room temperature. The mixture was cooled to 0° C., quenched with saturated sodium carbonate solution, filtered, washed with water and dried to obtain 8-bromo-7-chloro-1,5-naphthyridine-2-carbonitrile (350 mg).

### Intermediate 47: 8-bromo-7-fluoro-1,5-naphthyridine-2-carbonitrile

A mixture of ethyl 6-cyano-4-hydroxy-1,5-naphthyridine-3-carboxylate (337.5 g) (WO 2002024684) in POCl₃ (2 kg) was stirred at room temperature for 2 h. After most of the POCl₃ was removed under reduced pressure, the residue was poured into ice-water (10 L) containing NH₄OH (2 L). The mixture was extracted with EtOAc (3×10 L) and the combined extracts were washed with brine, dried over anhydrous sodium sulfate, filtered and concentrated in vacuo to afford ethyl 4-chloro-6-cyano-1,5-naphthyridine-3-carboxylate (250 g) as a dark-brown solid. To a suspension of the compound above (935 g) in THF (14 L) was added 2M NaOH (2.6 L) at room temperature. After stirring overnight at room temperature, the mixture was neutralized with 2M HCl to pH 6-7. After evaporating under reduced pressure, the pH was adjusted to pH 2 with 2M HCl. The solid was filtered, washed with water, and dried to a dark-brown solid of 4-chloro-6-cyano-1,5-naphthyridine-3-carboxylic acid (774 g).

To a suspension of the compound above (342 g) in DMF (4.9 L) were added triethylamine (1.8 L), tBu-OH (2.1 L) and DPPA (341 mL) at room temperature, and the resulted mixture was heated at 100° C. for 1 hour. After the mixture was cooled to room temperature, the reaction mixture was concentrated in vacuo. The residue was diluted with ethylacetate, washed with saturated NaHCO₃ solution. The aqueous layer was extracted with ethylacetate (2 times). The combined organic layers were washed with brine, dried (Na₂SO₄), filtered and concentrated. The residue was purified by flash chromatography to afford tert-butyl (4-chloro-6-methoxy-1,5-naphthyridin-3-yl)carbamate as an off white solid (310 g).

To a solution of tert-butyl (4-chloro-6-methoxy-1,5-naphthyridin-3-yl)carbamate (315 g) in methylene chloride (3 L) in an ice bath was added TFA (2.5 kg). After warming to room temperature over two hours, the mixture was concentrated and the residue was partitioned with chloroform and water. The mixture was basified with solid Na₂CO₃ to pH 9. The resulting solid was filtered and washed with water, and dried under vacuum to afford 4-chloro-6-methoxy-1,5-naphthyridin-3-amine (190 g) as a light-brown solid.

To a solution of 4-chloro-6-methoxy-1,5-naphthyridin-3-amine (40 g) in THF (500 mL) at 5° C. was added 48% HBF₄ (200 mL). The resulting mixture was cooled to −10° C. and a solution of NaNO₂ (13.8 g) in water (15 mL) was added. After the mixture was stirred at −5 to −10° C. for 2 hours, the solid was filtered, suspended in ether, filtered again, and dried under vacuum to afford 4-chloro-6-methoxy-1,5-naphthyridine-3-diazonium tetrafluoroborate (61 g) as a light yellow solid.

To a pre-heated decalin solution (1.2 L, 175-180° C.) was added the compound above (61 g) in small portions. The resulting mixture was stirred at 175-180° C. for 20 min. and then cooled to room temperature in an ice-bath. The solvents were decanted and evaporated under high vacuum to give a light-yellow solid. This solid and the black residue in the flask were dissolved in acetone and adsorbed on silica gel and subjected to flash chromatography to afford 8-chloro-7-fluoro-2-methoxy-1,5-naphthyridine as a white solid (21 g).

8-Chloro-7-fluoro-2-methoxy-[1,5]naphthyridine (1.2 g) was heated at reflux in HCl (6M, 50 mL) for 1 hour. The mixture was cooled to 0° C. and pH was adjusted to 7 with sodium hydroxide (50%). Solids were filtered, washed with water and dried in vacuo to give 8-chloro-7-fluoro-2-hydroxy-[1,5]naphthyridine (1.0 g).

To a solution of 8-chloro-7-fluoro-2-hydroxy-[1,5]naphthyridine (1.03 g) in methylene chloride (30 mL) at 0° C. was added DMAP (150 mg) and 2,6-lutidine (10.1 mL) followed by trifluoromethanesulfonic anhydride (1.3 mL). The mixture was stirred at 0° C. for 10 minutes and then stirred at room temperature for 1 hour. The mixture was poured into saturated ammonium chloride solution, washed with brine, dried over sodium sulfate and concentrated. Purification on silica gel (0-5% methanol in methylene chloride) gave 8-Chloro-7-fluoro-2-trifluoromethanesulfonate-[1,5]naphthyridine in quantitative yield.

In a 20 mL vessel 8-chloro-7-fluoro-2-trifluoromethanesulfonate-[1,5]naphthyridine (2.0 g) in DMF (20 mL) was added zinc cyanide (1.7 g) and tetrakis (triphenylphosphine)palladium(0) (500 mg). The mixture was heated at 115° C. for 20 minutes and then the mixture was cooled to room temperature, diluted with water and ethyl acetate, washed with saturated sodium bicarbonate solution and brine, dried on sodium sulfate and concentrated. Purification on silica gel column (0-5% methanol in methylene chloride) gave product (800 mg).

8-Chloro-7-fluoro-2-cyano-[1,5]naphthyridine (800 mg) was refluxed in acetic acid (30 mL) overnight. The mixture was filtered and washed with saturated sodium bicarbonate solution and water, and dried in vacuo to give product (200 mg).

To a solution of 8-hydroxy-7-fluoro-2-cyano-[1,5]naphthyridine (200 mg) in DMF (20 mL) at 0° C. was added phosphorous tribromide (0.170 mL). The ice bath was removed and the mixture was then stirred at room temperature for 1 hour. The mixture was then cooled to 0° C., poured onto saturated sodium carbonate solution, washed with water and diethyl ether, and dried in vacuo to give 8-bromo-7-fluoro-1,5-naphthyridine-2-carbonitrile (60 mg).

### Examples 39-41

The following compounds were synthesized in an analogous method to Example 24 except that ZnBr₂ was excluded from the preparation of Intermediate 38 to yield the des-halogen material, which was carried through all the subsequent reactions.

| | | | ES | |
|---|---|---|---|---|
| Example | Compound | ¹H NMR | (M + H)⁺ | aldehyde |
| 39 | (3S,6S)-N-(2,3- | (300 MHz, DMSO-D6) δ | 436 | 2,3-dihydro[1,4]dioxino[2,3- |
| | dihydro[1,4]dioxi- | ppm 1.46-1.75 (m, 2 H) | | c]pyridine-7-carbaldehyde |
| | no[2,3-c]pyridin-7- | 1.94-2.18 (m, 2 H) 2.20-2.33 | | |
| | ylmethyl)-6-[2-(6- | (m, 2 H) 2.95-3.10 | | |
| | methoxy-1,5- | (m, 2 H) 3.17-3.31 | | |
| | naphthyridin-4- | (m, 2 H) 3.40-3.54 (m, | | |
| | yl)ethyl]piperidin- | J = 11.68 Hz, 1 H) 3.56-3.68 | | |
| | 3-amine | (m, J = 11.11 Hz, 1 | | |
| | | H) 4.06 (s, 3 H) 4.25 (s, | | |
| | | 2 H) 4.36 (dd, J = 15.45, | | |
| | | 4.90 Hz, 4 H) 7.16 (s, 1 | | |
| | | H) 7.29 (d, J = 9.23 Hz, 1 | | |
| | | H) 7.62 (d, J = 4.52 Hz, 1 | | |
| | | H) 8.19 (s, 1 H) 8.28 (d, | | |
| | | J = 9.04 Hz, 1 H) 8.73 (d, | | |
| | | J = 4.33 Hz, 1 H) 9.37-9.68 | | |
| | | (m, 2 H) | | |
| 40 | (3S,6S)-N-[(2E)-3- | (400 MHz, DMSO-D6) δ | 439 | (2E)-3-(2,5- |
| | (2,5- | ppm 1.66 (m, 2 H) 2.03 | | difluorophenyl)acrylaldehyde |
| | difluorophenyl)prop- | (m, 1 H) 2.17 (m, 1 H) | | (FR2872164) |
| | 2-en-1-yl]-6-[2- | 2.24-2.34 (m, 2 H) 3.04 | | |
| | (6-methoxy-1,5- | (m, 1 H) 3.13 (m, 1 H) | | |
| | naphthyridin-4- | 3.21-3.33 (m, 2 H) 3.60 | | |
| | yl)ethyl]piperidin- | (m, 1 H) 3.91 (m, 2 H) | | |
| | 3-amine | 4.09 (s, 3 H) 6.47-6.56 | | |
| | | (m, 1 H) 6.97 (d, | | |
| | | J = 16.17 Hz, 1H) 7.21-7.29 | | |
| | | (m, 1 H) 7.30-7.37 | | |
| | | (m, 2 H) 7.50 (ddd, | | |
| | | J = 9.28, 6.00, 3.16 Hz, 1 | | |
| | | H) 7.70 (d, J = 4.55 Hz, 1 | | |
| | | H) 8.33 (d, J = 8.84 Hz, 1 | | |
| | | H) 8.79 (d, J = 4.55 Hz, 1 | | |
| | | H) 9.58 (m, 2 H) 9.72 | | |
| | | (m, 1 H) | | |
| 41 | 6-[({(3S,6S)-6-[2- | (400 MHz, DMSO-D6) δ | 449 | 3-oxo-3,4-dihydro-2H- |
| | (6-methoxy-1,5- | ppm 1.65 (m, 1 H) 1.98-2.09 | | pyrido[3,2-b][1,4]oxazine-6- |
| | naphthyridin-4- | (m, 1 H) 2.11-2.23 | | carbaldehyde |
| | yl)ethyl]piperidin- | (m, 1 H) 2.33 (m, 2 H) | | |
| | 3- | 3.06 (s, 1 H) 3.20-3.32 | | |
| | yl}amino)methyl]- | (m, 1 H) 3.68 (m, 1 H) | | |
| | 2H-pyrido[3,2- | 4.09 (s, 3 H) 4.25 (m, 2 | | |
| | b][1,4]oxazin- | H) 4.71 (s, 2 H) 7.22 (d, | | |
| | 3(4H)-one | J = 8.08 Hz, 1 H) 7.33 (d, | | |
| | | J = 9.09 Hz, 1 H) 7.47 (d, | | |
| | | J = 8.08 Hz, 1 H) 7.67 (d, | | |
| | | J = 4.55 Hz, 1 H) 8.31 (d, | | |
| | | J = 9.09 Hz, 1 H) 8.77 (d, | | |
| | | J = 4.55 Hz, 1 H) 9.59 (m, | | |
| | | 2 H) 11.36 (s, 1 H) | | |

### Example 42

### (3R,6R)-6-[2-(3-chloro-6-methoxy-1,5-naphthyridin-4-yl)ethyl]-N-(2,3-dihydro[1,4]dioxino[2,3-c]pyridin-7-ylmethyl)-1-methylpiperidin-3-amine

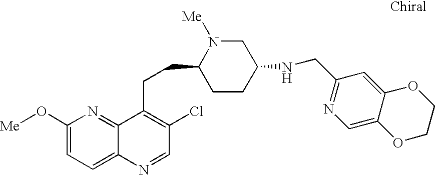

Example 33 (99 mg) was heated at reflux with LAH (0.340 mL) in THF (5 mL) for 2 hours. The reaction was concentrated, purified by HPLC (A: 0.1% TFA/water; B: 0.1% TFA/acetonitrile) and dried on a lyophilizer to give product. (21.7 mg).

¹H NMR (300 MHz, CHLOROFORM-D) δ ppm 1.77-2.17 (m, 3H) 2.15-2.30 (m, J=5.65 Hz, 1H) 2.34-2.55 (m, J=12.43 Hz, 2H) 2.92 (s, 3H) 3.13 (s, 1H) 3.26-3.55 (m, 3H) 3.75-3.94 (m, J=6.41 Hz, 1H) 4.06 (s, 3H) 4.44 (s, 2H) 4.53 (d, J=14.88 Hz, 4H) 7.19 (d, J=9.04 Hz, 1H) 7.46 (s, 1H) 8.08-8.21 (m, 1H) 8.27 (d, J=9.23 Hz, 1H) 8.67-8.79 (m, 1H),

ES (M+H)⁺=484.

### Examples 43

### 6-({[(3S,6S)-6-[2-(3-chloro-6-methoxy-1,5-naphthyridin-4-yl)ethyl]-1-(methylsulfonyl)piperidin-3-yl]amino}methyl)-2H-pyrido[3,2-b][1,4]oxazin-3(4H)-one

To a suspension of tert-butyl (2S,5S)-5-azido-2-[2-(3-fluoro-6-methoxy-1,5-naphthyridin-4-yl)ethyl]piperidine-1-carboxylate (Intermediate 39 from Example 24) (125 mg, 0.328 mmol) in THF (8 mL) was added diisopropylethyl amine (DIEA) (0.126 mL, 0.72 mmol) and methanesulfonyl chloride (0.30 mL). After stirring at room temperature overnight, the mixture was washed with water and extracted with ethyl acetate, dried over sodium sulfate, filtered and concentrated to give 130 mg of yellow oil of 8-{2-[(2S,5S)-5-azido-1-(methylsulfonyl)piperidin-2-yl]ethyl}-7-chloro-2-methoxy-1,5-naphthyridine.

Following similar procedures found in Example 24, 8-{2-[(2S,5S)-5-azido-1-(methylsulfonyl)piperidin-2-yl]ethyl}-7-chloro-2-methoxy-1,5-naphthyridine was reduced (see Intermediate 38 for conditions) and reductively aminated (see Intermediate 37 for conditions) to yield the named compound.

¹H NMR (300 MHz, DMSO-D6) δ ppm 1.73-1.89 (m, 2H) 1.93-2.03 (m, J=7.72 Hz, 2H) 2.03-2.20 (m, J=21.10 Hz, 2H) 2.98 (s, 3H) 3.16-3.34 (m, 3H) 3.80 (d, J=14.88 Hz, 1H) 4.05 (s, 3H) 4.20 (s, 2H) 4.69 (s, 2H) 7.16 (d, J=8.10 Hz, 1H) 7.31 (d, J=9.04 Hz, 1H) 7.46 (d, J=8.10 Hz, 1H) 8.30 (d, J=9.04 Hz, 1H) 8.78 (s, 1H) 8.82-9.09 (m, 2H) 11.35 (s, 1H).

ES (M+H)⁺=561.

### Example 44

### 6-({[(3R,6R)-6-[2-(3-chloro-6-methoxy-1,5-naphthyridin-4-yl)ethyl]-1-(methylsulfonyl)piperidin-3-yl amino}methyl)-2H-pyrido[3,2-b][1,4]oxazin-3(4H)-one

Prepared in a similar fashion as Example 43 starting from (R)-(−) methyl-2-pyrrolidone-5-carboxylate.

¹H NMR (300 MHz, DMSO-D6) δ ppm 1.51 (dd, J=51.05, 13.19 Hz, 2H) 1.67-1.85 (m, 2H) 1.89-2.21 (m, 2H) 2.63 (s, 1H) 3.12 (s, 3H) 3.15 (s, 2H) 3.56-3.68 (m, 1H) 3.74 (d, J=13.56 Hz, 2H) 3.97 (s, 2H) 4.03 (s, 3H) 4.60 (s, 2H) 6.91-7.09 (m, 1H) 7.17-7.38 (m, 2 H) 8.27 (d, J=9.04 Hz, 1H) 8.74 (s, 1H) 11.19 (s, 1H).

ES (M+H)⁺=561.

### Example 45

### 6-[({(3S,6S)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one

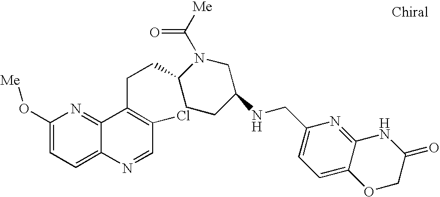

To a suspension of tert-butyl (2S,5S)-5-azido-2-[2-(3-fluoro-6-methoxy-1,5-naphthyridin-4-yl)ethyl]piperidine-1-carboxylate (Intermediate 39 from Example 24) (185 mg) in methylene chloride (3 mL) was added triethylamine (0.25 mL) and acetyl chloride (0.07 mL). After stirring at room temperature overnight, the mixture was diluted with methylene chloride, washed with a saturated solution of NaHCO₃ and brine, dried (Na₂SO₄), filtered and concentrated yielding 0.20 g of 8-{2-[(2S,5S)-1-acetyl-5-azidopiperidin-2-yl]ethyl}-7-chloro-2-methoxy-1,5-naphthyridine,

ES (M+H)⁺=389.

Following similar procedures found in Example 24, 8-{2-[(2S,5S)-1-acetyl-5-azidopiperidin-2-yl]ethyl}-7-chloro-2-methoxy-1,5-naphthyridine was reduced (see Intermediate 38 for conditions) and reductively aininated (see Intermediate 37 for conditions) to yield the named compound.

¹H NMR (300 MHz, MeOD) δ ppm 1.80 (m, 1H) 1.90-2.06 (m, 2H) 2.16 (s, 3H) 3.13 (m, 1H) 3.53 (m, 1H) 4.03 (s, 3H) 4.28 (m, 3H) 4.64 (s, 2H) 7.07 (d, J=8.10 Hz, 1H) 7.15 (d, J=9.23 Hz, 1H) 7.31 (d, J=8.10 Hz, 1H) 8.11 (d, J=9.04 Hz, 1H).

ES (M+H)⁺=525.

### Example 46

### 6-[({(3R,6R)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one

Prepared in a similar fashion as Example 45 starting from (R)-(−) methyl-2-pyrrolidone-5-carboxylate.

¹H NMR (300 MHz, CHLOROFORM-D) δ ppm 1.71 (m, 1H) 1.98 (m, 3H) 2.30 (s, 3H) 2.71 (m, 1H) 2.95 (m, 2H) 3.29 (m, 2H) 4.05 (s, 3H) 4.27 (m, 1H) 4.37 (m, 1H) 4.50 (s, 2H) 4.99 (m, 1H) 7.04-7.20 (m, 3H) 8.20 (d, J=8.85 Hz, 1H) 8.67 (s, 1H).

ES (M+H)⁺=525.

### Example 47

### ((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetic acid

To a suspension of tert-butyl (2S,5S)-5-azido-2-[2-(3-fluoro-6-methoxy-1,5-naphthyridin-4-yl)ethyl]piperidine-1-carboxylate (Intermediate 39 from Example 24) (274 mg) in THF (4 mL) was added DIEA (0.30 mL) and t-butyl bromoacetate (0.12 mL). After stirring at room temperature overnight, additional t-butyl bromoacetate (0.30 mL) was added. After stirring over the weekend, the mixture was diluted with ethylacetate and water. The organic solution was collected, washed with brine, dried (Na₂SO₄), filtered and concentrated yielding tert-butyl {(2S,5S)-5-azido-2-[2-(3-chloro-6-methoxy-1,5-naphthyridin-4-yl)ethyl]piperidin-1-yl}acetate.

Following similar procedures found in Example 24, tert-butyl {(2S,5S)-5-azido-2-[2-(3-chloro-6-methoxy-1,5-naphthyridin-4-yl)ethyl]piperidin-1-yl}acetate was reduced (see Intermediate 38 for conditions) and reductively aminated (see Intermediate 37 for conditions) to yield 100 mg of tert-butyl ((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetate,

(M+H)⁺=597.

To a solution of tert-butyl ((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetate (100 mg) in dioxane (1 mL) and methanol (1 mL) was added 4 M HCl/dioxane (1 mL). After stirring for 6 hours, the reaction was quenched with 2N NaOH. The residue was concentrated and purified by Gilson (A: 0.1% TFA/water; B: 0.1% TFA/acetonitrile). The desired fractions were collected, reduced in vacuo, dissolved in a minimum of methanol and precipitated with diethyl ether. The white solid was filtered and washed with diethyl ether yielding 47 mg of the named compound.

¹H NMR (400 MHz, DMSO-D6) δ ppm 1.57 (m, 2H) 1.78 (m, 2H) 1.91 (m, 1H) 2.06 (m, 1H) 2.84 (m, 2H) 3.26 (m, 1H) 3.86 (m, 2H) 4.04 (s, 3H) 4.64 (s, 2H) 7.07 (d, J=8.08 Hz, 1H) 7.30 (d, J=8.84 Hz, 1H) 7.36 (d, J=8.08 Hz, 1H) 8.29 (d, J=9.09 Hz, 1H) 8.76 (s, 1H) 11.26 (s, 1H).

ES (M+H)⁺=541.

### Example 48

### ((2S,5S)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid

Prepared in a similar fashion as Example 47 except that (2E)-3-(2,5-difluorophenyl)acrylaldehyde was used during the reductive amination step (see Intermediate 37 for conditions).

¹H NMR (300 MHz, DMSO-D6) δ ppm 1.63-1.98 (m, 3H) 2.00-2.18 (m, J=20.25, 9.51 Hz, 1H) 2.21-2.46 (m, 2H) 3.13-3.27 (m, 2H) 3.27-3.44 (m, 3H) 3.85 (s, 2H) 4.06 (s, 3H) 4.12 (s, 2H) 6.40-6.61 (m, 1H) 6.94 (d, J=16.01 Hz, 1H) 7.17-7.37 (m, 2H) 7.42-7.55 (m, 1H) 8.30 (d, J=9.04 Hz, 1H) 8.78 (s, 1H) 9.68-9.96 (m, 1H).

ES (M+H)⁺=531.

### Example 49

### ((2R,5R)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid

Prepared in a similar fashion as Example 48 starting from (R)-(−) methyl-2-pyrrolidone-5-carboxylate.

¹H NMR (300 MHz, DMSO-D6) δ ppm 1.56-1.96 (m, 4H) 1.97-2.18 (m, 1H) 2.19-2.41 (m, 2H) 3.16-3.44 (m, 4H) 4.00-4.13 (m, 5H) 6.41-6.60 (m, 1H) 6.83-7.04 (m, 1H) 7.15-7.40 (m, 3H) 7.43-7.55 (m, 1H) 8.30 (d, J=9.04 Hz, 1H) 8.78 (s, 1H) 9.71 (s, 2H).

ES (M+H)⁺=531.

### Example 50

### 6-({([(3S,6S)-6-[2-(3-chloro-6-methoxy-1,5-naphthyridin-4-yl)ethyl]-1-(methoxyacetyl)piperidin-3-yl amino}methyl)-2H-pyrido[3,2-b][1,4]oxazin-3(4H)-one

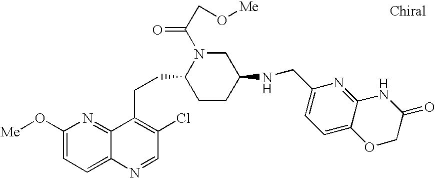

To a suspension of tert-butyl (2S,5S)-5-azido-2-[2-(3-fluoro-6-methoxy-1,5-naphthyridin-4-yl)ethyl]piperidine-1-carboxylate (Intermediate 39 from Example 24) (145 mg, 0.38 mmol) in THF (7.0 mL) was added DIEA (0.132 mL) and methoxy chloroacetate (0.07 mL). After stirring for 30 minutes at room temperature, the mixture was washed with water, extracted with ethyl acetate, dried over sodium sulfate and concentrated under reduces pressure to give an oil of 8-{2-[(2S,5S)-5-azido-1-(methoxyacetyl)piperidin-2-yl]ethyl}-7-chloro-2-methoxy-1,5-naphthyridine (190 mg).

Following similar procedures found in Example 24, 8-{2-[(2S,5s)-1-acetyl-5-azidopiperidin-2-yl]ethyl}-7-chloro-2-methoxy-1,5-naphthyridine was reduced (see Intermediate 38 for conditions) and reductively aminated (see Intermediate 37 for conditions) to yield the named compound.

¹H NMR (300 MHz, DMSO-D6) δ ppm 0.19 (dd, J=177.18, 88.82 Hz, 1H) 1.13-1.31 (m, 1H) 1.41-1.63 (m, J=20.91 Hz, 2H) 1.67-1.83 (m, J=11.68 Hz, 2H) 1.84-2.10 (m, 3H) 3.27 (s, 6H) 3.92 (s, 2H) 4.04 (s, 4H) 4.64 (s, 2H) 7.08 (d, J=6.41 Hz, 1H) 7.31 (d, J=8.48 Hz, 1H) 7.33-7.45 (m, 1H) 8.28 (d, J=8.48 Hz, 1H) 8.76 (s, 1H) 11.26 (s, 1H).

ES (M+H)⁺=555.

### Example 51

### 2-((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)-2-oxoethyl acetate

To a suspension of tert-butyl (2S,5S)-5-azido-2-[2-(3-fluoro-6-methoxy-1,5-naphthyridin-4-yl)ethyl]piperidine-1-carboxylate (Intermediate 39 from Example 24) (180 mg) in THF (7.0 mL) was added DIEA (0.197 mL) and acetoxyacetyl chloride (0.1.1 mL) and the mixture was stirred at room temperature overnight. The mixture was the washed with water, extracted with ethyl acetate, dried over sodium sulfate, filtered and concentrated to give crude 2-{(2S,5S)-5-azido-2-[2-(3-chloro-6-methoxy-1,5-naphthyridin-4-yl)ethyl]piperidin-1-yl}-2-oxoethyl acetate as oil (300 mg).

Following similar procedures found in Example 24, 8-{2-[(2S,S)-1-acetyl-5-azidopiperidin-2-yl]ethyl}-7-chloro-2-methoxy-1,5-naphthyridine was reduced (see Intermediate 38 for conditions) and reductively aminated (see Intermediate 37 for conditions) to yield the named compound.

¹H NMR (300 MHz, CHLOROFORM-D) δ ppm 1.35-1.51 (m, 1H) 1.71-1.87 (m, 1H) 1.98-2.10 (m, 3H) 2.13 (s, 2H) 2.19 (s, 3H) 3.12 (d, J=14.32 Hz, 1H) 3.37 (t, J=8.10 Hz, 2H) 3.53 (s, 1H) 4.06 (s, 4H) 4.27 (s, 2H) 4.50 (s, 1H) 4.59 (d, J=5.65 Hz, 2H) 4.79 (d, J=14.51 Hz, 1H) 4.96 (dd, J=26.66, 14.41 Hz, 2H) 7.01 (d, J=7.91 Hz, 1H) 7.13 (d, J=9.04 Hz, 1H) 7.23 (s, 1H) 8.18 (d, J=9.04 Hz, 1H) 8.68 (s, 1H).

ES (M+H)⁺=583.

### Example 52

### 6-[({(3S,6S)-6-[2-(3-chloro-6-methoxy-1,5-naphthyridin-4-yl)ethyl]-1-glycoloylpiperidin-3-yl}amino)methyl]-2H-pyrido[3,2-b][1,4]oxazin-3(4H)-one

2-((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)-2-oxoethyl acetate (Example 51) (100 mg) was stirred in ammonia/methanol for 48 hours. The reaction was filtered and washed with methanol to yield the named compound (60 mg).

¹H NMR (300 MHz, DMSO-D6) δ ppm 1.35-1.62 (m, 2H) 1.69-1.85 (m, 2H) 1.86-1.99 (m, 2H) 2.70 (s, 1H) 3.10-3.27 (m, 3H) 3.41-3.54 (m, J=13.00 Hz, 1H) 3.66 (s, 2H) 4.04 (s, 3H) 4.08 (s, 1H) 4.40-4.50 (m, J=4.90 Hz, 1H) 4.59 (s, 2H) 4.74 (s, 1H) 7.00 (d, J=8.10 Hz, 1H) 7.23-7.35 (m, 2H) 8.27 (d, J=9.04 Hz, 1H) 8.74 (s, 1H) 11.17 (s, 1H).

ES (M+H)⁺=541.

### Example 53

### (3R,6S)—N-(2,3-dihydro [1,4]dioxino [2,3-c]pyridin-7-ylmethyl)-6-[2-(3-fluoro-6-methoxy-1,5-naphthyridin-4-yl)ethyl]piperidin-3-amine

Following similar procedures found in Example 24, tert-butyl (2S,5R)-5-azido-2-[2-(3-fluoro-6-methoxy-1,5-naphthyridin-4-yl)ethyl]piperidine-1-carboxylate (see Intermediate 38 minor material) was reduced (see Intermediate 38 for conditions) and reductively aminated (see Intermediate 37 for conditions) and deprotected to yield the named compound.

¹H NMR (400 MHz, CHLOROFORM-D) δ ppm 1.86 (m, 1H) 2.05 (m, 2H) 2.17 (m, 2H) 2.36-2.46 (m, 1H) 3.15 (m, 2H) 3.28 (m, 3H) 3.50 (m, 1H) 3.83 (m, 1H) 4.07 (s, 3H) 4.15 (m, 2H) 4.29 (m, 2H) 4.38 (s, 2H) 7.05 (d, J=9.09 Hz, 1H) 7.09 (s, 1H) 8.15 (m, 2H) 8.58 (s, 1H).

ES (M+H)⁺=454.

All cited publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula II 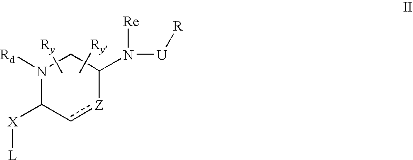
or a pharmaceutically acceptable salt thereof, wherein:
L is 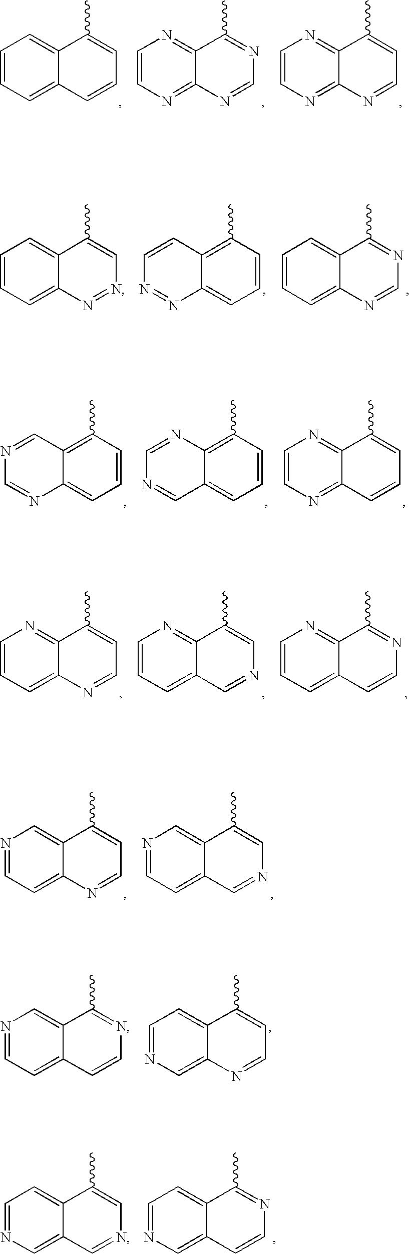 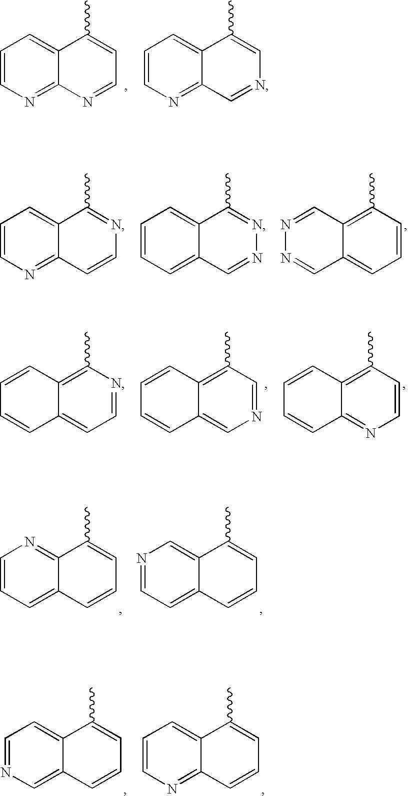
wherein [symbol] indicates the point of attachment, and wherein
each L is optionally substituted with 1, 2, or 3 groups independently selected from the group consisting of H, halo, cyano, nitro, (C₁-C₆)alkanoyl, carboxy, (C₁-C₆)alkoxycarbonyl, (C₁-C₆)alkyl, hydroxyl, halo(C₁-C₆)alkyl, halo(C₁-C₆)alkoxy, (C₁-C₆)alkoxy, NHCO—(C₁-C₆)alkyl, SO₂(C₁-C₆)alkyl, SO₂NH(C₁-C₆)alkyl, or SO₂N((C₁-C₆)alkyl)₂;
X is NHCO, N(C₁-C₆)alkylCO, CO—CR₁R₂, CR₁R₂—CO, NR₁SO₂, CR₁R₂—SO₂ or CR₁R₂—CR₁R₂, wherein R₁ and R₂ at each occurrence is independently H, hydroxyl, (C₁-C₆)alkyl, halogen, halo(C₁-C₆)alkyl, aryl, or heteroaryl; or
X is O—CR₁R₂, NR₁—CR₁R₂, wherein R₁ and R₂ are H, (C₁-C₆)alkyl, halo(C₁-C₆)alkyl, aryl, or heteroaryl;
Z is absent or is C; [symbol]
is a bond or is absent;
R_{d} is H, (C₁-C₆)alkyl, (C₂-C₆)alkenyl, 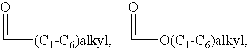
hydroxy(C₁-C₃)alkyl, CONH₂, CO₂H, —CH₂CH₂CO₂H, —CH₂CONH₂, —CH₂CO₂H, —CONH(C₁-C₆)alkyl, trifluoromethyl, S(O)ₓR₁, wherein x is 1 or 2, provided that when R_{d} is H and Z is C, [symbol] is a bond;
Ry and Ry′ are each independently halogen, (C₁-C₆)alkyl, (C₁-C₆)alkoxy, hydroxyl, CONH₂, CO₂H, —CH₂CONH₂, —CH₂CO₂H, —CONHCH₃ or amino, provided that when Ry and Ry′ are hydroxyl, amino, or halogen, they are not attached to the same carbon, or when Ry and Ry′ are attached to the same carbon, they form C═O;
Rₑ is H, (C₁-C₆)alkyl, 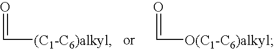
U is CH₂, CH₂CH₂, CH═CH, or C≡C and wherein each hydrogen may be optionally replaced by fluoro or (C₁-C₆)alkyl;
R is an optionally substituted aryl or ortho-fused bicyclic heteroaryl, or when U is ethylene, ethenyl, or ethynyl, R is optionally substituted aryl or heteroaryl, or is heteroaryl(C₁-C₆)alkyloxy, heteroaryl(C₁-C₆)alkylthio, heteroaryl(C₁-C₆)alkylsulfinyl, heteroaryl(C₁-C₆)alkylsulfonyl, heteroaryl(C₁-C₆)alkylamino.

2. The compound of claim 1 which is a compound of formula II-1 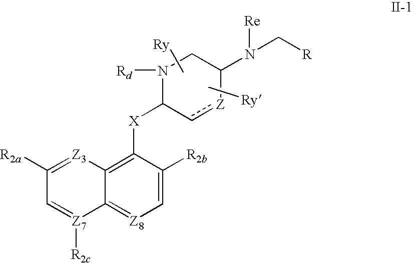
wherein:
Z₃, Z₇, and Z₈ are C or N provided that when Z₇ is N, R_{2c} is absent, and
R₂ₐ is H, cyano, (C₁-C₆)alkyl, hydroxyl, halo, halo(C₁-C₆)alkoxy, (C₁-C₆)alkoxy.

3. The compound of claim 1, wherein 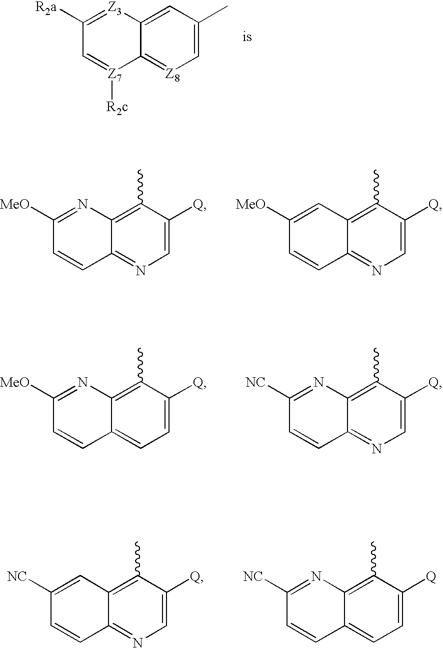
wherein [symbol] indicates the point of attachment and Q is hydrogen, fluoro, or chloro.

4. The compound of claim 1, wherein X is NHCO, CO—CH₂, CH₂CH₂, O—CH₂, CHOHCH₂, or NHCH₂;
Ry and Ry′ each independently are H or (C₁-C₆)alkyl or taken together with the carbon to which they are attached form C═O;
R is benzo[1,2,5]thiadiazol-5-yl. Other specific values for R include 4H-benzo[1,4]thiazin-3-one-6-yl, 2,3-dihydro-benzo[1,4]dioxin-6-yl, benzo[1,2,3]thiadiazol-5-yl, 3-oxo-3,4-dihydro-2H-benzo[1,4]oxazin-6-yl, 7-fluoro-3-oxo-3,4-dihydro-2H-benzo[1,4]oxazin-6-yl, 2-oxo-2,3-dihydro-1H-pyrido[2,3-b][1,4]thiazin-7-yl, 2,3-dihydro-1,4]dioxino[2,3-c]pyridin-7-yl, 3-oxo-3,4-dihydro-2H-pyrido[3,2-b]1,4]oxazin-6-yl, [1,2,3]thiadiazolo 5,4-b]pyridin-6-yl, 3-oxo-3,4-dihydro-2H-pyrido[3,2-b][14]thiazin-6-yl, 7-chloro-3-oxo-3,4-dihydro-2H-pyrido[3,2-b][1,4]thiazin-6-yl, 7-fluoro-3-oxo-3,4-dihydro-2H-pyrido[3,2-b][1,4]thiazin-6-yl, 2-thienylthio-methyl, or 2,5-difluorophenylvinyl.

5. The compound of claim 1 which is a compound of formula II-2. 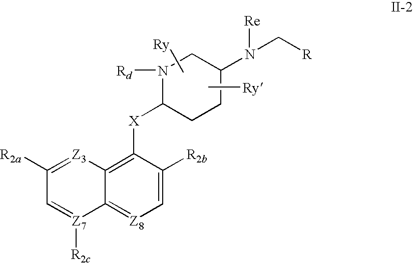
wherein R_{d} is (C₁-C₆)alkyl, 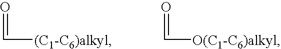
methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

6. The compound of claim 1 which is a compound of formula II-3. 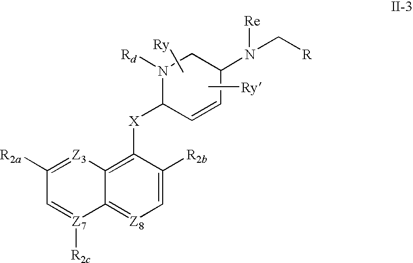
wherein
R_{d} is H, (C₁-C₆)alkyl, 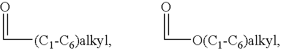
methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

7. The compound of claim 1 which is a compound of formula II-4 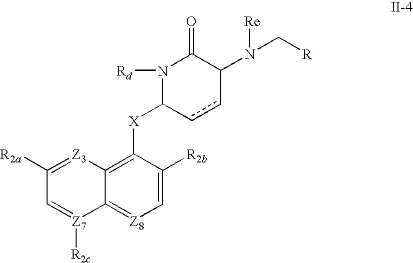
wherein R_{d} is H, (C₁-C₆)alkyl, carboxy(C₁-C₆)alkyl, 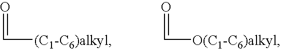
methyl CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

8. The compound of claim 1 which is a compound of formula II-5. 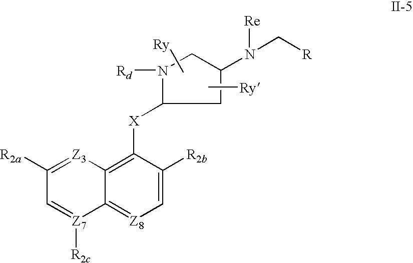
wherein R_{d}H, is (C₁-C₆)alkyl, carboxy(C₁-C₆)alkyl, 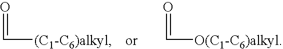
methyl, CONH₂, CO₂H, —CH₂CH₂CO₂H, —CH₂CONH₂, —CH₂CO₂H, —CONHCH₃, SO₂Me, COCH₃, COCH₂OMe, or COCH₂OH.

9. A compound which is:
(2S,5R)-5-[(2,3-dihydro-1,4-benzodioxin-6-ylmethyl)amino]-N-(6-methoxy-1,5-naphthyridin-4-yl)-6-oxopiperidine-2-carboxamide;
5-[(2,3-dihydro-1,4-benzodioxin-6-ylmethyl)amino]-N-(6-methoxy-1,5-naphthyridin-4-yl)-6-oxopiperidine-2-carboxamide;
(2S,5S)-5-[(2,3-dihydro-1,4-benzodioxin-6-ylmethyl)amino]-N-(6-methoxy-1,5-naphthyridin-4-yl)-6-oxopiperidine-2-carboxamide;
(4R)-4-[(2,3-dihydro[1,4]dioxino[2,3-c]pyridin-7-ylmethyl)amino]-N-(8-fluoro-6-methoxyquinolin-4-yl)-L-prolinamide;
(4R)—N-(6-cyano-1,7-naphthyridin-4-yl)-4-[(2,3-dihydro[1,4]dioxino[2,3-c]pyridin-7-ylmethyl)amino]-L-prolinamide;
(4R)-4-[(2,3-dihydro[1,4]dioxino[2,3-c]pyridin-7-ylmethyl)amino]-N-(6-methoxy-1,5-naphthyridin-4-yl)-L-prolinamide;
(4R)—N-(2-cyanoquinolin-8-yl)-4-[(2,3-dihydro[1,4]dioxino[2,3-c]pyridin-7-ylmethyl)amino]-L-prolinamide;
(3R,6R)-6-[2-(3-chloro-6-methoxy-1,5-naphthyridin-4-yl)ethyl]-N-(2,3-dihydro[1,4]dioxino[2,3-c]pyridin-7-ylmethyl)-1-methylpiperidin-3-amine;
6-[({(3S,6S)-6-[2-(3-chloro-6-methoxy-1,5-naphthyridin-4-yl)ethyl]-1-glycoloylpiperidin-3-yl}amino)methyl]-2H-pyrido[3,2-b][1,4]oxazin-3(4H)-one;
6-[({(3R,6R)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)acetic acid;
6-[({(3S,6S)-1-acetyl-6-[2-(3-chloro-6-methoxy-1,5-naphthyridin-4-yl)ethyl]piperidin-3-yl}amino)methyl]-2H-pyrido[3,2-b][1,4]oxazin-3(4H)-one;
((2R,5R)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
6-({[(3S,6S)-6-[2-(3-chloro-6-methoxy-1,5-naphthyridin-4-yl)ethyl]-1-(methoxyacetyl)piperidin-3-yl]amino}methyl)-2H-pyrido[3,2-b][1,4]oxazin-3(4H)-one;
((2S,5S)-2-[2-(3-chloro-6-methoxy-1,5-naphthyridin-4-yl)ethyl]-5-{[(2E)-3-(2,5-difluorophenyl)prop-2-en-1-yl]amino}piperidin-1-yl)acetic acid;
tert-butyl (2R,5R)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidine-1-carboxylate;
2-((2S,5S)-2-[2-(3-chloro-6-methoxy-1,5-naphthyridin-4-yl)ethyl]-5-{[(3-oxo-3,4-dihydro-2H-pyrido[3,2-b][1,4]oxazin-6-yl)methyl]amino}piperidin-1-yl)-2-oxoethyl acetate; or
(2S,5S)-5-[(2,3-dihydro-1,4-benzodioxin-6-ylmethyl)(methyl)amino]-N-(6-methoxy-1,5-naphthyridin-4-yl)-1-methylpiperidine-2-carboxamide,
or a pharmaceutically acceptable salt thereof.

10. A pharmaceutical composition comprising a compound of claim 1 admixed with a pharmaceutically acceptable adjuvant, carrier, or excipient.

11. A method of treating a bacterial infection comprising administering a therapeutically effective amount of a compound of claim 1 to a mammal in need thereof.

12. A method of treating a bacterial infection in a warm-blooded animal, such as a human being, in need of such treatment, which comprises administering to said animal an effective amount of a compound of claim 1 or a pharmaceutically-acceptable salt thereof.

13. A method for inhibiting bacterial DNA gyrase in a warm-blooded animal, such as a human being, in need of such treatment which comprises administering to said animal an effective amount of a compound of claim 1 or a pharmaceutically acceptable salt.

14. A compound of claim 1 and pharmaceutically acceptable salts thereof for use as a medicament.

15. A compound of claim 1 or a pharmaceutically acceptable salt thereof in the manufacture of a medicament for use in the production of an anti-bacterial effect in a warm-blooded animal such as a human being.

16. A compound of claim 1 or a pharmaceutically acceptable salt thereof in the manufacture of a medicament for use in the treatment of a bacterial infection in a warm-blooded animal such as a human being.

17. A process for making a compound of claim 1 said process comprising one of the following approaches: 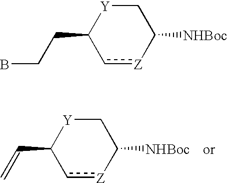
(a) Pd-catalyzed coupling of 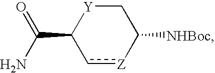
wherein Y is N-PG, wherein PG is a protecting group, with 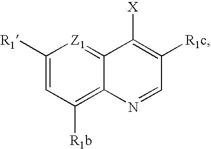
wherein X is a leaving group selected from halo or trifluoromethylsulfonyloxy, followed by removal of the BOC group and addition of U—R via reductive amination;
(b) Coupling of 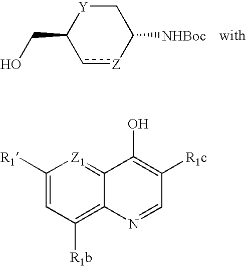
under Mitsunobu conditions followed by removal of the BOC group and addition of U—R via reductive amination; or
(c) Amide formation using 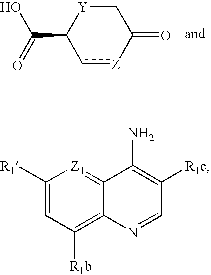
followed by addition of U—R via reductive amination.
